SEQUENCE LISTING (51 sequences)

SEQ ID NO: 1 - protein, 337 aa, Homo sapiens [sequence omitted]

SEQ ID NO: 2 - protein, 350 aa, Homo sapiens
MNSFNYTTPDYGHYDDKDTLDLNTPVDKTSNTLRVPDILALVIFAVVFLVGVLGNALVVWVTAFEAKRTINAIWFLNLAVADFLSCLALPILFTSIVQHHHWPFGGAACSILPSLILLNMYASILLLATISADRFLLVFKPIWCQNFRGAGLAWIACAVAWGLALLLTIPSFLYRVVREEYFPPKVLCGVDYSHDKRRERAVAIVRLVLGFLWPLLTLTICYTFILLRTWSRRATRSTKTLKVVVAVVASFFIFWLPYQVTGIMMSFLEPSSPTFLLLNKLDSLCVSFAYINCCINPIIYVVAGQGFQGRLRKSLPSLLRNVLTEESVVRESKSFTRSTVDTMAQKTQAV

SEQ ID NO: 3 - protein, 482 aa, Homo sapiens
MASFSAETNSTDLLSQPWNEPPVILSMVILSLTFLLGLPGNGLVLWVAGLKMQRTVNTIWFLHLTLADLLCCLSLPFSLAHLALQGQWPYGRFLCKLIPSIIVLNMFASVFLLTAISLDRCLVVFKPIWCQNHRNVGMACSICGCIWVVAFVMCIPVFVYREIFTTDNHNRCGYKFGLSSSLDYPDFYGDPLENRSLENIVQPPGEMNDRLDPSSFQTNDHPWTVPTVFQPQTFQRPSADSLPRGSARLTSQNLYSNVFKPADVVSPKIPSGFPIEDHETSPLDNSDAFLSTHLKLFPSASSNSFYESELPQGFQDYYNLGQFTDDDQVPTPLVAITITRLVVGFLLPSVIMIACYSFIVFRMQRGRFAKSQSKTFRVAVVVVAVFLVCWTPYHIFGVLSLLTDPETPLGKTLMSWDHVCIALASANSCFNPFLYALLGKDFRKKARQSIQGILEAAFSEELTRSTHCPSNNVISERNSTTV

SEQ ID NO: 4 - protein, 344 aa, Mus musculus
MMNHTTSEYYDYEYDHEHYSDLPDVPVDCPAGTCFTSDVYLIVLLVLYAAVFLVGVPGNTLVAWVTWKESRHRLGASWFLHLTMADLLCCVSLPFLAVPIAQKGHWPYGAAGCWLLSSITILSMYASVLLLTGLSGDLFLLAFRPSWKGADHRTFGVRVVQASSWMLGLLLTVPSAVYRRLLQEHYPPRLVCGIDYGGSVSAEVAITTVRFLFGFLGPLVFMAGCHGILQRQMARRHWPLGTAVVVGFFICWTPYHVLRVIIAAAPPHSLLLARVLEAEPLFNGLALAHSALNPIMFLYFGRKQLCKSLQAACHWALRDPQDEESAVTKVSISTSHEMVSEMPV

SEQ ID NO: 5 - protein, 350 aa, Mus musculus
MDPIDNSSFEINYDHYGTMDPNIPADGIHLPKRQPGDVAALIIYSVVFLVGVPGNALVVWVTAFEARRAVNAIWFLNLAVADLLSCLALPVLFTTVLNHNYWYFDATACIVLPSLILLNMYASILLLATISADRFLLVFKPIWCQKVRGTGLAWMACGVAWVLALLLTIPSFVYREAYKDFYSEHTVCGINYGGGSFPKEKAVAILRLMVGFVLPLLTLNICYTFLLLRTWSRKATRSTKTLKVVMAVVICFFIFWLPYQVTGVMIAWLPPSSPTLKRVEKLNSLCVSLAYINCCVNPIIYVMAGQGFHGRLLRSLPSIIRNALSEDSVGRDSKTFTPSTTDTSTRKSQA

SEQ ID NO: 6 - protein, 477 aa, Mus musculus
MESFDADTNSTDLHSRPLFQPQDIASMVILGLTCLLGLLGNGLVLWVAGVKMKTTVNTVWFLHLTLADFLCCLSLPFSLAHLILQGHWPYGLFLCKLIPSIIILNMFASVFLLTAISLDRCLIVHKPIWCQNHRNVRTAFAICGCVWVVAFVMCVPVFVYRDLFIMDNRSICRYNFDSSRSYDYWDYVYKLSLPESNSTDNSTAQLTGHMNDRSAPSSVQARDYFWTVTTALQSQPFLTSPEDSFSLDSANQQPHYGGKPPNVLTAAVPSGFPVEDRKSNTLNADAFLSAHTELFPTASSGHLYPYDFQGDYVDQFTYDNHVPTPLMAITITRLVVGFLVPFFIMVICYSLIVFRMRKTNFTKSRNKTFRVAVAVVTVFFICWTPYHLVGVLLLITDPESSLGEAVMSWDHMSIALASANSCFNPFLYALLGKDFRKKARQSIKGILEAAFSEELTHSTNCTQDKASSKRNNMSTDV

SEQ ID NO: 7 - protein, 14 aa, Artificial Sequence
  Description of Artificial Sequence Synthetic peptide
  Conserved Sequence Fragment characteristic for human and mouse C5L2, C5AR1 and C3AR
FLVGVPGNAMVAWV

SEQ ID NO: 8 - protein, 10 aa, Artificial Sequence
  Description of Artificial Sequence Synthetic peptide
  Conserved Sequence Fragment characteristic for human and mouse C5L2, C5AR1 and C3AR
ADLLCCLSLP

SEQ ID NO: 9 - protein, 9 aa, Artificial Sequence
  Description of Artificial Sequence Synthetic peptide
  Conserved Sequence Fragment characteristic for human and mouse C5L2, C5AR1 and C3AR
MYASVLLLA

SEQ ID NO: 10 - protein, 9 aa, Artificial Sequence
  Description of Artificial Sequence Synthetic peptide
  Conserved Sequence Fragment characteristic for human and mouse C5L2, C5AR1 and C3AR
LALLLTVPS

SEQ ID NO: 11 - protein, 8 aa, Artificial Sequence
  Description of Artificial Sequence Synthetic peptide
  Conserved Sequence Fragment characteristic for human and mouse C5L2, C5AR1 and C3AR
FFVCWAPY

SEQ ID NO: 12 - protein, 6 aa, Artificial Sequence
  Description of Artificial Sequence Synthetic peptide
  Conserved Sequence Fragment characteristic for human and mouse C5L2, C5AR1 and C3AR
GHWPYG

SEQ ID NO: 13 - protein, 11 aa, Artificial Sequence
  Description of Artificial Sequence Synthetic peptide
  Conserved Sequence Fragment characteristic for human and mouse C5L2, C5AR1 and C3AR
YSDLSDRPVDC

SEQ ID NO: 14 - protein, 11 aa, Artificial Sequence
  Description of Artificial Sequence Synthetic peptide
  Conserved Sequence Fragment characteristic for human and mouse C5L2, C5AR1 and C3AR
YSDLPDVPVDC

SEQ ID NO: 15 - protein, 9 aa, Artificial Sequence
  Description of Artificial Sequence Synthetic peptide
  Conserved Sequence Fragment characteristic for human and mouse C5L2, C5AR1 and C3AR
TLDLNTPVD

SEQ ID NO: 16 - protein, 9 aa, Artificial Sequence
  Description of Artificial Sequence Synthetic peptide
  Conserved Sequence Fragment characteristic for human and mouse C5L2, C5AR1 and C3AR
TMDPNIPAD

SEQ ID NO: 17 - protein, 10 aa, Artificial Sequence
  Description of Artificial Sequence Synthetic peptide
  Conserved Sequence Fragment characteristic for human and mouse C5L2, C5AR1 and C3AR
PLVAITITRL

SEQ ID NO: 18 - protein, 23 aa, Artificial Sequence
  Description of Artificial Sequence Synthetic peptide
  N-terminal fragment of human C5L2
MGNDSVSYEYGDYSDLSDRPVDC

SEQ ID NO: 19 - protein, 29 aa, Artificial Sequence
  Description of Artificial Sequence Synthetic peptide
  N-terminal fragment of mouse C5L2
MMNHTTSEYYDYEYDHEHYSDLPDVPVDC

SEQ ID NO: 20 - protein, 74 aa, Homo sapiens
TLQKKIEEIAAKYKHSVVKKCCYDGACVNNDETCEQRAARISLGPRCIKAFTECCVVASQLRANISHKDMQLGR

SEQ ID NO: 21 - protein, 73 aa, Homo sapiens
TLQKKIEEIAAKYKHSVVKKCCYDGACVNNDETCEQRAARISLGPRCIKAFTECCVVASQLRANISHKDMQLG

SEQ ID NO: 22 - protein, 14 aa, Artificial Sequence
  Description of Artificial Sequence Synthetic peptide
  Artificial Sequence - Sequence 22
TLQKKIEEIAAKYK

SEQ ID NO: 23 - protein, 13 aa, Artificial Sequence
  Description of Artificial Sequence Synthetic peptide
  Artificial Sequence - Prior Art
HSVVKKCCYDGAC

SEQ ID NO: 24 - protein, 5 aa, Artificial Sequence
  Description of Artificial Sequence Synthetic peptide
  Artificial Sequence - Prior Art
VNNDE

SEQ ID NO: 25 - protein, 8 aa, Artificial Sequence
  Description of Artificial Sequence Synthetic peptide
  Artificial Sequence - Prior Art
TCEQRAAR

SEQ ID NO: 26 - protein, 4 aa, Artificial Sequence
  Description of Artificial Sequence Synthetic peptide
  Artificial Sequence
ISLG

SEQ ID NO: 27 - protein, 22 aa, Artificial Sequence
  Description of Artificial Sequence Synthetic peptide
  Artificial Sequence - Prior Art
PRCIKAFTECCVVASQLRANIS

SEQ ID NO: 28 - protein, 7 aa, Artificial Sequence
  Description of Artificial Sequence Synthetic peptide
  Artificial Sequence - Prior Art
HKDMQLG

SEQ ID NO: 29 - protein, 8 aa, Artificial Sequence
  Description of Artificial Sequence Synthetic peptide
  Artificial Sequence - Prior Art
HKDMQLGR

SEQ ID NO: 30 - protein, 14 aa, Artificial Sequence
  Description of Artificial Sequence Synthetic peptide
  Artificial Sequence - Prior Art
CCYDGACVNNDETC

SEQ ID NO: 31 - protein, 33 aa, Artificial Sequence
  Description of Artificial Sequence Synthetic polypeptide
  Artificial Sequence - Prior Art
CYDGACVNNDETCEQRAARISLGPRCIKAFTEC

SEQ ID NO: 32 - protein, 22 aa, Artificial Sequence
  Description of Artificial Sequence Synthetic peptide
  Artificial Sequence - Prior Art
CEQRAARISLGPRCIKAFTECC

SEQ ID NO: 33 - protein, 18 aa, Artificial Sequence
  Description of Artificial Sequence Synthetic peptide
  Artificial Sequence - Prior Art
YDGACVNNDETCEQRAAR

SEQ ID NO: 34 - protein, 18 aa, Artificial Sequence
  Description of Artificial Sequence Synthetic peptide
  Artificial Sequence - Prior Art
CYDGACVNNDETCEQRAA

SEQ ID NO: 35 - protein, 9 aa, Artificial Sequence
  Description of Artificial Sequence Synthetic peptide
  Artificial Sequence - Prior art
  MOD_RES (1)..(1): N, H, D, F, K, Y or T
  MOD_RES (2)..(2): D, L, Y or H
  MOD_RES (7)..(7): Q, E, or K
  MOD_RES (9)..(9): A, V, or L
XXETCEXRX

SEQ ID NO: 36 - protein, 7 aa, Artificial Sequence
  Description of Artificial Sequence Synthetic peptide
  Artificial Sequence - prior art
  MOD_RES (1)..(1): S, H, P, or N
  MOD_RES (2)..(2): H or N
  MOD_RES (4)..(4): D, N, H, P, or G
  MOD_RES (5)..(5): M, L, I, or V
  MOD_RES (6)..(6): Q, L, or I
XXKXXXL

SEQ ID NO: 37 - protein, 7 aa, Artificial Sequence
  Description of Artificial Sequence Synthetic peptide
  Artificial Sequence - Prior Art
  MOD_RES (1)..(1): S, H, P, or N
  MOD_RES (2)..(2): H or N
  MOD_RES (4)..(4): D, N, H, P, or G
  MOD_RES (5)..(5): M, L, I, or V
  MOD_RES (6)..(6): Q, L, or I
XXKXXXI

SEQ ID NO: 38 - protein, 9 aa, Artificial Sequence
  Description of Artificial Sequence Synthetic peptide
  Artificial Sequence - Prior Art
NDETCEQRA

SEQ ID NO: 39 - protein, 7 aa, Artificial Sequence
  Description of Artificial Sequence Synthetic peptide
  Artificial Sequence - Prior Art
SHKDMQL

SEQ ID NO: 40 - protein, 7 aa, Artificial Sequence
  Description of Artificial Sequence Synthetic peptide
  Artificial Sequence - Prior Art
DETCEQR

SEQ ID NO: 41 - DNA, 40 bp, Artificial Sequence
  Description of Artificial Sequence Synthetic oligonucleotide
  Description of Combined DNA/RNA Molecule Synthetic oligonucleotide
  Artificial Sequence from US 20120065254
gcgaugugguggugaaggguuguugggugucgacgcacgc

SEQ ID NO: 42 - protein, 7 aa, Artificial Sequence
  Description of Artificial Sequence Synthetic peptide
  C5a amino acid including sequence from Yatime, L. et al., Nat Commun. 2015
KKCCYDG

SEQ ID NO: 43 - protein, 77 aa, Homo sapiens
SVQLTEKRMDKVGKYPKELRKCCEDGMRENPMRFSCQRRTRFISLGEACKKVFLDCCNYITELRRQHARASHLGLAR

SEQ ID NO: 44 - protein, 8 aa, Artificial Sequence
  Description of Artificial Sequence Synthetic peptide
  Artificial Sequence
ASHLGLAR

SEQ ID NO: 45 - protein, 9 aa, Artificial Sequence
  Description of Artificial Sequence Synthetic peptide
  Artificial Sequence
ASHLGLARG

SEQ ID NO: 46 - protein, 13 aa, Artificial Sequence
  Description of Artificial Sequence Synthetic peptide
  Artificial Sequence
RQHARASHLGLAR

SEQ ID NO: 47 - protein, 14 aa, Artificial Sequence
  Description of Artificial Sequence Synthetic peptide
  Artificial Sequence
RQHARASHLGLARG

SEQ ID NO: 48 - protein, 77 aa, Homo sapiens
NVNFQKAINEKLGQYASPTAKRCCQDGVTRLPMMRSCEQRAARVQQPDCREPFLSCCQFAESLRKKSRDKGQAGLQR

SEQ ID NO: 49 - protein, 380 aa, Homo sapiens
TLEIPGNSDPNMIPDGDFNSYVRVTASDPLDTLGSEGALSPGGVASLLRLPRGCGEQTMIYLAPTLAASRYLDKTEQWSTLPPETKDHAVDLIQKGYMRIQQFRKADGSYAAWLSRDSSTWLTAFVLKVLSLAQEQVGGSPEKLQETSNWLLSQQQADGSFQDPCPVLDRSMQGGLVGNDETVALTAFVTIALHHGLAVFQDEGAEPLKQRVEASISKANSFLGEKASAGLLGAHAAAITAYALSLTKAPVDLLGVAHNNLMAMAQETGDNLYWGSVTGSQSNAVSPTPAPRNPSDPMPQAPALWIETTAYALLHLLLHEGKAEMADQASAWLTRQGSFQGGFRSTQDTVIALDALSAYWIASHTTEERGLNVTLSSTGR

SEQ ID NO: 50 - protein, 6 aa, Artificial Sequence
  Description of Artificial Sequence Synthetic peptide
  Artificial Sequence 50
PCPVLD

SEQ ID NO: 51 - protein, 5 aa, Artificial Sequence
  Description of Artificial Sequence Synthetic peptide
LGLAR

(19) United States
(12) Patent Application Publication
(10) Pub. No.: US 2023/0183325 A1
(43) Pub. Date: Jun. 15, 2023

(54) COMPLEMENT ANAPHYLATOXIN BINDERS AND THEIR USE IN TREATMENT OF A SUBJECT HAVING AN OCULAR WOUND AND/OR FIBROSIS

(71) Applicant: CHARITE - UNIVERSITATSMEDIZIN BERLIN, Berlin (DE)

(72) Inventors: Tobias BROCKMANN, Berlin (DE); Eckart BERTELMANN, Berlin (DE); Uwe PLEYER, Berlin (DE)

(73) Assignee: CHARITE - UNIVERSITATSMEDIZIN BERLIN, Berlin (DE)

(21) Appl. No.: 17/253,958

(22) PCT Filed: Jun. 20, 2019
(86) PCT No.: PCT/EP2019/066419

§ 371 Date: Dec. 18, 2020

(30) Foreign Application Priority Data
Jun. 21, 2018 (EP) 18179178.1

(51) Int. Cl. C07K 16/18; C12N 15/115; A61P 27/02

(52) U.S. Cl. CPC C07K 16/18; C12N 15/115; A61P 27/02

(57) ABSTRACT

Subject matter of the present invention is a binder, e.g. protein or protein fragment, binding to complement-anaphylatoxin C5a and/or C3a and/or C4a and thereby inhibiting the activity of C5a and/or C3a and/or C4a for use in the treatment of a subject having an ocular wound and/or fibrosis.

Subject matter of the present invention is a binder, e.g. a protein or protein fragment or peptide, binding to complement anaphylatoxin C5a and/or C3a and/or C4a and thereby inhibiting the activity of C5a and/or C3a and/or C4a for use in the treatment of a subject having an ocular wound and/or fibrosis.

### PRIOR ART

Degenerative eye disorders, which are associated to a severe loss of visual acuity very often are the result of misguided angiogenesis or wound healing/fibrogenesis (Friedlander M. J Clin Invest. 2007). While the treatment of vascular eye disorders was substantially improved by profound research and the introduction of anti-VEGF therapeutics (Vascular Endothelial Growth Factor, VEGF), (Lim L S et al. Lancet 2012; Feigl B. Prog Retin Eye Res 2009; Joussen A M et al. FASEB J 2004) the treatment of fibrotic eye disorders is still lacking on therapeutic approaches.

Misguided wound healing and fibrogenesis is of outmost relevance in particular on the cornea. Corneal fibrosis results in a loss of optical transparency that substantially impedes vision and may result in blindness of the affected eye. Corneal scars can occur on base of a corneal herpetic infection, microbial keratitis, mechanic or chemical affection, stromal keratopathies, persistent corneal edema due to endothelial decompensation or corneal graft failure. Today, in most cases a penetrating corneal transplantation is the only therapeutic option to restore vision. In this regard, the number of performed corneal transplantations and the number of severe corneal complications, associated with corneal fibrosis, due to contact lenses or due to corneal laser refractive surgeries is increasing. Notwithstanding the above, the life-time risk to suffer a relevant ocular trauma, with corneal affection accounts for 20%. (Ljubimov A V et al. Prog Retin Eye Res 2015) Current therapeutic options to inhibit ocular fibrogenesis are very limited and primarily refer to corticosteroids and ciclosporin A (CSA). Both substances possess a non-specific efficacy, which is accompanied with various adverse effects. In this regard, corticosteroids induce cataract development and intraocular pressure elevation but also evoke systemic adverse events, such as the Cushing syndrome and alterations of blood parameters (glucose). CSA has a slow onset of action, which usually responds too slowly to prevent fibrosis, therefore CSA is not feasible for an acute treatment, its topical application is accompanied with stinging and redness of the eyes and also evokes systemic adverse events, in particular arterial hypertension.

However, this therapeutic dilemma not only relates to the cornea, as mentioned in the examples above, but also to tissue fibrosis in various conditions of misled wound healing and scarring in eye diseases, involving ocular fibroblast and myofibroblasts, which occur in the conjunctiva, sclera, iris, trabecular meshwork, vitreous, retina, choroid and optic nerve head. Furthermore, fundamental pathophysiologic processes involved in fibrosis and scarring, related to fibroblast activation and/or differentiation, are likewise of relevance for fibrotic diseases of the lung, liver, kidney, pancreas, heart, skin and vascular system. Against this background, the establishment of new therapeutic options for the treatment of ocular fibrosis and superordinate fibrotic conditions is of considerable clinical importance.

The physiological wound healing intervenes several tissue processes and follows a sequence of cell migration and/or transformation, proliferation and modulation of the extracellular matrix; (Ljubimov A V et al. Prog Retin Eye Res 2015) whereas activated fibroblasts and myofibroblasts are the key mediators. (Gabbiani G., J Pathol 2003) During the regular course of wound healing, reversible protein depositions are accumulated within the extracellular matrix. (Wynn T A et al. Nat Med 2012) Yet, in the context of fibrotic remodeling, which is triggered by a dysregulation of pro- and anti-fibrotic cascades, a permanent myofibroblasts activation emerges that may lead to a constant and irreversible deposition of matrix proteins, such as collagen, fibronectin and proteoglycans. (Medzhitov R. Cell 2010; Wynn T A, J Pathol. 2008).

On the basis of the aforementioned, the inhibition of myofibroblasts and their activation may selectively direct wound-healing processes to regular clearance-mechanisms and thereby prevent tissue fibrosis and scarring. However, regarding the inhibition of ocular myofibroblasts, anatomic particularities of the eye have to be considered. First, the blood-ocular barrier prevents the efficacy of systemically applied inhibitors/modulators, especially those based on proteins/peptides. Second, the direct application (e.g. topical, in the form of eye drops) requires the penetration of the inhibitor/modulator into the tissue that is intended to be treated. Therefore the inhibitors/modulators need to as small as to penetrate into the conjunctiva, sclera, iris, trabecular meshwork, vitreous, retina, choroid, or even the optic nerve head. Proteins with a molecular weight of 28-67 kDa are able to penetrate through the cornea with an intact corneal epithelium into the anterior chamber, while proteins with a molecular weight of 60-90 kDa are able to penetrate through the cornea into the anterior chamber after removal of the corneal epithelium. (Thiel M A et al. Clin Exp Immunol 2002) Conventional therapeutic approaches of specific inhibitors, such as monoclonal antibodies (anti-VEGF antibody, bevacizumab: 149 kDa), do not fulfill these conditions.

It was the object of the present invention to provide a treatment of a subject having an ocular wound or fibrosis that overcomes the shortcomings of the prior art methods.

Therefore, the aim of the present invention is to provide a substance that inhibits the process of fibroblast/myofibroblast activation and/or transdifferentiation, i.e. at least essentially inhibits the process of fibroblast/myofibroblast activation and/or transdifferentiation and has preferably a molecular weight less than 90 kDa, preferably less than 80 kDa or less, preferably less than 70 kDa or less, more preferably less than 60 kDa or less, more preferably less than 50 kDa or less, more preferably less than 45 kDa or less, more preferably less than 40 kDa or less, even more preferably less than 35 kDa or less, even more preferably less than 30 kDa or less, even more preferably less than 25 kDa or less, even more preferably less than 20 kDa or less, even more preferably less than 15 kDa or less, and even more preferably less than 10 kDa or less.

Subject matter of the present invention is a binder, in particular a protein or protein fragment, binding to complement-anaphylatoxin C5a and/or C3a and/or C4a and preferably thereby inhibiting the activity of C5a and/or C3a and/or C4a for use in the treatment of a subject having an ocular wound or fibrosis.

Inhibiting the activity of C5a and/or C3a and/or C4a means inhibiting essentially the action of C5a and/or C3a and/or C4a by binding to C5a and/or C3a and/or C4a.

Subject matter of the present invention is a binder for use in the treatment of a subject having an ocular wound or fibrosis wherein said binder is administered to promote wound healing, in particular corneal wound healing.

A binder maybe selected from the group comprising a protein or a fragment thereof, a peptide, a non-IgG scaffold in particular an aptamer, oligonucleotides, an antibody or antibody-like proteins, peptidomimetics or a fragment thereof.

Antibodies, antibody-like proteins or binders, as described above, may bind to several overlapping peptide fragments of a complement component C5a protein (e.g., several overlapping fragments of a human C5a protein having the amino acid sequence depicted in SEQ ID No.: 20 or SEQ ID No.: 21), wherein overlapping means the overlapping of the targeted amino acid sequences of the antibody, antibody-like protein or binder and the specific peptide fragments. The antibodies, antibody-like proteins or binders may also bind only to a human C5a at an epitope within or overlapping with a fragment of the protein having the amino acid sequence, according to SEQ ID No's.: 22-34 (see e.g., Cooketal. (2010) Acta Cryst D66:190-197 and as described in US 2016/0159892). Furthermore, the antibody, antibody-like protein or binder may also bind to an epitope of C5a formed by amino acid sequences according to SEQ ID No's: 35-40 (SEQ ID No.: 35: X₁X₂ETCEX₃RX₄, SEQ ID No.: 36: X₅X₆KX₇X₈X₉L and SEQ ID No.: 37: X₅X₆KX₇X₈X₉I), wherein X₁ is selected from the group consisting of N, H, D, F, K, Y, and T; X₂ is selected from the group consisting of D, L, Y, and H; X₃ is selected from the group consisting of Q, E, and K; X₄ is selected from the group consisting of A, V, and L; X₅ is selected from the group consisting of S, H, P, and N; X₆ is selected from the group consisting of H and N; X₇ is selected from the group consisting of D, N, H, P, and G; X₈ is selected from the group consisting of M, L, I, and V; and X₉ is selected from the group consisting of Q, L, and I (as described in US 2012/0231008, US 2017/0002067, WO 2011/063980 and U.S. Pat. No. 8,802,096).

Antibodies, antibody-like proteins or binders, as described above, may bind to several overlapping peptide fragments of a complement component C3a protein (e.g., several overlapping fragments of a human C3a protein having the amino acid sequence depicted in SEQ ID No.: 43). The antibodies, antibody-like proteins or binders may also bind only to a human C3a at an epitope within or overlapping with a fragment of the protein having the amino acid sequence, according to SEQ ID No's.: 44-47 (see e.g., Hugli T E. J Biol Chem. 1975; Hugli T E et al. PNAS 1977; Payan D et al. J. Exp Med. 1982).

Antibodies, antibody-like proteins or binders, as described above, may bind to several overlapping peptide fragments of a complement component C4a protein (e.g., several overlapping fragments of a human C4a protein having the amino acid sequence depicted in SEQ ID No.: 48 or SEQ ID No.: 49). The antibodies, antibody-like proteins or binders may also bind only to a human C4a at an epitope within or overlapping with a fragment of the protein having the amino acid sequence, according to SEQ ID No.: 50 (see e.g., Yu C Y et al. EMBO J. 1986; Nettesheim D. G. et al. PNAS 1988).

A peptide is defined as a compound consisting of at least two amino acids in which the carboxyl group of one acid is linked to the amino group of the other, which can be created by peptide synthesis. Thus, as defined for this invention a peptide may have from 2 to 50 amino acids. A protein comprises more than 50 amino acids, according to the definition of this invention.

A protein is defined as a macromolecule consisting of one or more chains of amino acids, or peptides, linked by peptide bonds, which can be created by protein ligation of two or more peptides, by recombinant expression or by protein biosynthesis.

A protein fragment is defined as a section of an amino acids sequence that derives from a protein that served as template.

An antibody according to the present invention is a protein including one or more polypeptides substantially encoded by immunoglobulin genes that specifically binds an antigen. The recognized immunoglobulin genes include the kappa, lambda, alpha (IgA), gamma (IgG₁, IgG₂, IgG₃, IgG₄), delta (IgD), epsilon (IgE) and mu (IgM) constant region genes, as well as the myriad immunoglobulin variable region genes. Full-length immunoglobulin light chains are generally about 25 kDa or 214 amino acids in length. Full-length immunoglobulin heavy chains are generally about 50 kDa or 446 amino acid in length. Light chains are encoded by a variable region gene at the NH2-terminus (about 110 amino acids in length) and a kappa or lambda constant region gene at the COOH-terminus. Heavy chains are similarly encoded by a variable region gene (about 116 amino acids in length) and one of the other constant region genes.

The basic structural unit of an antibody is generally a tetramer that consists of two identical pairs of immunoglobulin chains, each pair having one light and one heavy chain. In each pair, the light and heavy chain variable regions bind to an antigen, and the constant regions mediate effector functions. Immunoglobulins also exist in a variety of other forms including, for example, Fv, Fab, and (Fab′)₂, as well as bifunctional hybrid antibodies and single chains (e.g., Lanzavecchia et al., Eur. J. Immunol. 17:105,1987; Huston et al., Proc. Natl. Acad. Sci. U.S.A., 85:5879-5883, 1988; Bird et al., Science 242:423-426, 1988; Hood et al., Immunology, Benjamin, N. Y., 2nd ed., 1984; Hunkapiller and Hood, Nature 323:15-16, 1986). An immunoglobulin light or heavy chain variable region includes a framework region interrupted by three hypervariable regions, also called complementarity determining regions (CDR's) (see, Sequences of Proteins of Immunological Interest, E. Kabat et al., U.S. Department of Health and Human Services, 1983). As noted above, the CDRs are primarily responsible for binding to an epitope of an antigen. An immune complex is an antibody, such as a monoclonal antibody, chimeric antibody, humanized antibody or human antibody, or functional antibody fragment, specifically bound to the antigen.

Chimeric antibodies are antibodies whose light and heavy chain genes have been constructed, typically by genetic engineering, from immunoglobulin variable and constant region genes belonging to different species. For example, the variable segments of the genes from a mouse monoclonal antibody can be joined to human constant segments, such as kappa and gamma 1 or gamma 3. In one example, a therapeutic chimeric antibody is thus a hybrid protein composed of the variable or antigen-binding domain from a mouse antibody and the constant or effector domain from a human antibody, although other mammalian species can be used, or the variable region can be produced by molecular techniques. Methods of making chimeric antibodies are well known in the art, e.g., see U.S. Pat. No. 5,807,715. A “humanized” immunoglobulin is an immunoglobulin including a human framework region and one or more CDRs from a non-human (such as a mouse, rat, or synthetic) immunoglobulin. The non-human immunoglobulin providing the CDRs is termed a “donor” and the human immunoglobulin providing the framework is termed an “acceptor”. In one embodiment, all the CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions, which have substantially no effect on antigen binding or other immunoglobulin functions. Exemplary conservative substitutions are those such as gly, ala; val, ile, leu; asp, glu; asn, gln; ser, thr; lys, arg; and phe, tyr. Humanized immunoglobulins can be constructed by means of genetic engineering (e.g., see U.S. Pat. No. 5,585,089). A human antibody is an antibody wherein the light and heavy chain genes are of human origin. Human antibodies can be generated using methods known in the art. Human antibodies can be produced by immortalizing a human B cell secreting the antibody of interest Immortalization can be accomplished, for example, by EBV infection or by fusing a human B cell with a myeloma or hybridoma cell to produce a trioma cell. Human antibodies can also be produced by phage display methods (see, e.g., Dower et al., PCT Publication No. WO91/17271; McCafferty et al., PCT Publication No. WO92/001047; and Winter, PCT Publication No. WO92/20791), or selected from a human combinatorial monoclonal antibody library (see the Morphosys website). Human antibodies can also be prepared by using transgenic animals carrying a human immunoglobulin gene (for example, see Lonberg et al., PCT Publication No. WO93/12227; and Kucherlapati, PCT Publication No. WO91/10741).

Thus, the antibody according to the present invention may have the formats known in the art. Examples are human antibodies, monoclonal antibodies, humanized antibodies, chimeric antibodies, CDR-grafted antibodies. In a preferred embodiment antibodies according to the present invention are recombinantly produced antibodies as e.g. IgG, a typical full-length immunoglobulin, or antibody fragments containing at least the F-variable domain of heavy and/or light chain as e.g. chemically coupled antibodies (fragment antigen binding) including but not limited to Fab-fragments including Fab minibodies, single chain Fab antibody, monovalent Fab antibody with epitope tags, e.g. Fab-V5Sx2; bivalent Fab (mini-antibody) dimerized with the CH3 domain; bivalent Fab or multivalent Fab, e.g. formed via multimerization with the aid of a heterologous domain, e.g. via dimerization of dHLX domains, e.g. Fab-dHLX-FSx2; F(ab′)2-fragments, scFv-fragments, multimerized multivalent or/and multispecific scFv-fragments, bivalent and/or bispecific diabodies, BITE® (bispecific T-cell engager), trifunctional antibodies, polyvalent antibodies, e.g. from a different class than G; single-domain antibodies, e.g. nanobodies derived from camelid or fish immunoglobulins and numerous others.

In addition to antibodies other biopolymer scaffolds are well known in the art to complex a target molecule and have been used for the generation of highly target specific biopolymers. Examples are aptamers, spiegelmers, anticalins and conotoxins.

In a preferred embodiment the antibody format is selected from the group comprising Fv fragment, scFv fragment, Fab fragment, scFab fragment, (Fab)2 fragment and scFv-Fc Fusion protein. In another preferred embodiment the antibody format is selected from the group comprising scFab fragment, Fab fragment, scFv fragment and bioavailability optimized conjugates thereof, such as PEGylated fragments. One particular formats is the scFab format.

Non-Ig scaffolds may be protein scaffolds and may be used as antibody mimics as they are capable to bind to ligands or antigenes. Non-Ig scaffolds may be selected from the group comprising tetranectin-based non-Ig scaffolds (e.g. described in US 2010/0028995), fibronectin scaffolds (e.g. described in EP 1266 025; lipocalin-based scaffolds ((e.g. described in WO 2011/154420); ubiquitin scaffolds (e.g. described in WO 2011/073214), transferring scaffolds (e.g. described in US 2004/0023334), protein A scaffolds (e.g. described in EP 2231860), ankyrin repeat based scaffolds (e.g. described in WO 2010/060748), microproteins, preferably microproteins forming a cystine knot) scaffolds (e.g. described in EP 2314308), Fyn SH3 domain based scaffolds (e.g. described in WO 2011/023685) EGFR-A-domain based scaffolds (e.g. described in WO 2005/040229) and Kunitz domain based scaffolds (e.g. described in EP 1941867).

Non-immunoglobulin (Non-IgG) scaffolds are defined as small antibody alternatives. An aptamer is defined as a molecule that binds to a specific target and may consist of RNA and/or DNA and/or amino acids (peptide).

An aptamer, may relate to a nucleic acid molecule consisting of RNA and/or DNA, such as disclosed in SEQ ID No.: 41 (5′-GCGAU G(dU)GGU GGU(dG)(dA) AGGGU UGUUG GG(dU)G(dU) CGACG CA(dC)GC-3′) and as described in US 2012/0065254, capable of binding to C5a, whereas the binding site of C5a is comprising a C5a amino acid sequence including SEQ ID No.: 42 (see Yatime L. et al. Nat Commun. 2015).

In one embodiment of the invention antibodies according to the present invention may be produced as follows:

A Balb/c mouse was immunized with antigen-100 μg Peptide-BSA-Conjugate (BSA=bovine serum albumin) at day 0 and 14 (emulsified in 100 μl complete Freund's adjuvant) and 50 μg at day 21 and 28 (in 100 μl incomplete Freund's adjuvant). Three days before the fusion experiment was performed, the animal received 50 μg of the conjugate dissolved in 100 μl saline, given as one intraperitoneal and one intravenous injection.

Splenocytes from the immunized mouse and cells of the myeloma cell line SP2/0 were fused with 1 ml 50% polyethylene glycol for 30 s at 37° C. After washing, the cells were seeded in 96-well cell culture plates. Hybrid clones were selected by growing in HAT medium (RPMI (Roswell Park Memorial Institute) 1640 culture medium supplemented with 20% fetal calf serum and HAT-Supplement). After two weeks the HAT medium is replaced with HAT Medium for three passages followed by returning to the normal cell culture medium.

The cell culture supernatants were primary screened for antigen specific IgG antibodies three weeks after fusion. The positive tested microcultures were transferred into 24-well plates for propagation. After retesting, the selected cultures were cloned and recloned using the limiting-dilution technique and the isotypes were determined (see also Lane, R. D. (1985). A short-duration polyethylene glycol fusion technique for increasing production of monoclonal antibody-secreting hybridomas. J. Immunol. Meth. 81: 223-228; Ziegler, B. et al. (1996) Glutamate decarboxylase (GAD) is not detectable on the surface of rat islet cells examined by cytofluorometry and complement-dependent antibody-mediated cytotoxicity of monoclonal GAD antibodies, Horm. Metab. Res. 28: 11-15).

Antibodies may be produced by means of phage display according to the following procedure:

The human naive antibody gene libraries HAL7/8 were used for the isolation of recombinant single chain F-Variable domains (scFv) against peptide. The antibody gene libraries were screened with a panning strategy comprising the use of peptides containing a biotin tag linked via two different spacers to the peptide sequence. A mix of panning rounds using non-specifically bound antigen and streptavidin bound antigen were used to minimize background of non-specific binders. The eluted phages from the third round of panning have been used for the generation of monoclonal scFv expressing E. coli strains. Supernatant from the cultivation of these clonal strains has been directly used for an antigen ELISA testing (see Hust, M., Meyer, T., Voedisch, B., Rülker, T., Thie, H., El-Ghezal, A., Kirsch, M. I., Schütte, M., Helmsing, S., Meier, D., Schirrmann, T., Dübel, S., 2011. A human scFv antibody generation pipeline for proteome research. Journal of Biotechnology 152, 159-170; Schütte, M., Thullier, P., Pelat, T., Wezler, X., Rosenstock, P., Hinz, D., Kirsch, M. I., Hasenberg, M., Frank, R., Schirrmann, T., Gunzer, M., Hust, M., Dübel, S., 2009. Identification of a putative Crf splice variant and generation of recombinant antibodies for the specific detection of Aspergillus fumigatus. PLoS One 4, e6625).

Humanization of murine antibodies may be conducted according to the following procedure:

For humanization of an antibody of murine origin the antibody sequence is analyzed for the structural interaction of framework regions (FR) with the complementary determining regions (CDR) and the antigen. Based on structural modeling an appropriate FR of human origin is selected and the murine CDR sequences are transplanted into the human FR. Variations in the amino acid sequence of the CDRs or FRs may be introduced to regain structural interactions, which were abolished by the species switch for the FR sequences. This recovery of structural interactions may be achieved by random approach using phage display libraries or via directed approach guided by molecular modeling (see Almagro J C, Fransson J., 2008. Humanization of antibodies. Front Biosci. 2008 Jan. 1; 13:1619-33).

In a preferred embodiment the antibody format is selected from the group comprising Fv fragment, scFv fragment, Fab fragment, scFab fragment, F(ab)₂ fragment and scFv-Fc Fusion protein. In another preferred embodiment the antibody format is selected from the group comprising scFab fragment, Fab fragment, scFv fragment and bioavailability optimized conjugates thereof, such as PEGylated fragments. One of the most preferred formats is scFab format.

In one embodiment of the invention said binder, e.g. a protein or protein fragment thereof, according to the present invention binds to C5a and C3a and thereby inhibiting the activity of C5a and C3a

In one embodiment of the invention said binder, e.g. a protein or protein fragment according to the present invention binds to C5a and C4a and thereby inhibiting the activity of, C5a and C4a.

In one embodiment of the invention said binder, e.g. a protein or protein fragment according to the present invention binds to C3a and C4a and thereby inhibiting the activity of C3a and C4a.

In one embodiment of the invention said binder, e.g. a protein or protein fragment according to the present invention binds to C5a and C3a and C4a and thereby inhibiting the activity of C5a and C3a and C4a.

In one specific embodiment of the invention said binder, e.g. a protein or protein fragment is a soluble complement receptor protein or protein fragment. In one specific embodiment of the invention said protein or protein fragment/peptide is a recombinant soluble complement receptor protein or synthetic protein fragment/peptide.

A soluble receptor is defined as the extracellular portion of the receptor, (Fischer D G. Science 1993) in case of C3a it is the extracellular portion of the C3a anaphylatoxin chemotactic receptor (C3aR1), in case of C5a it is the extracellular portion of the C5a anaphylatoxin chemotactic receptor 1 and/or 2 (C5aR1/CD88 and C5aR2/C5L2). A separate specific C4a receptor is not known, therefore in case of C4a it is the extracellular portion of the C3a anaphylatoxin chemotactic receptor (C3aR1) and/or the C5a anaphylatoxin chemotactic receptor 1 and/or 2 (C5aR1/CD88 and/or C5aR2/C5L2).

In one embodiment of the invention said binder, e.g. protein or protein fragment/peptide, according to the present invention binds specifically to complement-anaphylatoxin C5a and/or C3a and/or C4a.

Receptor/ligand binding affinities of the anaphylatoxin chemotactic receptors (C3aR1, C5aR1/CD88 and C5aR2/C5L2) to their main ligands (C3a and C5a, respectively) and cross-reactivities to all other anaphylatoxins (C3a, C4a, C5a) are known state-of-art (Cain S A. et al. J Biol Chem. 2002, Kalant D. et al. J Biol Chem 2003, Okinaga S. et al. Biochemistry 2003). Relevant ligand binding sites within the amino acid sequences, which mainly contribute to extracellular and transmembrane domains, of the anaphylatoxin chemotactic receptors have been investigated and therefore are known state-of-art.

Regarding C3aR1, studies have shown that the large extracellular loop 2 domain plays an important role in ligand binding; furthermore the charged transmembrane residues Arg161, Arg340 and Asp417 are essential for ligand effector binding and/or signal coupling (Sun J. et al. Protein Sci. 1999).

Amino acid sequence depicted in SEQ ID No.: 17 covers amino acids 332-341, a fragment of the large extracellular loop 2 including Arg340, of the human C3aR1 (SEQ ID No.: 3), which has a 90% identity of the corresponding amino acid sequence of the mouse C3aR1 (SEQ ID No.: 6).

The receptor binding sites in human C3a have been well investigated and have been summarized by Sun et al. (Sun J et al. Protein Sci. 1999), as following: Human C3a is composed of 77 amino acids. The three-dimensional structure of C3a consists of a large globular core of four closely packed alpha-helices covalently linked by three disulfide bonds with a C-terminal flexible irregular structure (Huber R et al. Hoppe Seyler's Z Physiol Chem. 1980). The C-terminal region of C3a is folded in a pseudo-beta-turn and is stabilized by an adjacent alpha-helical segment according to NMR studies (Chazin W J et al. Biochemistry 1988). The C-terminal 21 residues fragment of C3a (i.e., C3a 57-77) has been shown to retain all of the biologic activities of the natural molecule (Lu Z X et al. J Biol Chem. 1984, Ember J A et al. Biochemistry 1991). Synthetic peptide analogs of C3a demonstrated that the primary effector binding site in C3a exists in the irregular C-terminal region (LGLAR sequence) (Caporale L H et al. J Biol Chem. 1980, Unson C G et al. Biochemistry 1984).

In one embodiment, the binder that is subject matter of the present invention may bind to said irregular C-terminal 21 residues fragment of C3a.

Regarding C5aR1/CD88, studies have shown that the extracellular N-terminus plays an important role in ligand binding, in particular the five aspartic acids within amino acids 2-22 are essential for ligand effector binding, and thereby contributes to at least 45% of the total binding energy of C5a (DeMartino J A. J Biol Chem. 1994) and the extracellular loop 2 and 3 domains are relevant for ligand effector binding that interact with the C-terminus of C5a (Siciliano S J et al. PNAS. 1994, Monk P N et al. J Biol Chem. 1995). Furthermore, Tyr11 and Tyr14 are posttranslationally sulfated, which is critical for C5aR1 to bind C5a (Farzan M et al. J Exp Med. 2001). Known binding sites, functions and structures of C5a anaphylatoxin chemotactic receptors are summarized in a comprehensive review (Monk P N et al. Br J Pharmacol. 2007).

Amino acid sequence depicted in SEQ ID No.: 15 covers amino acids 19-27, a fragment of the N-terminus including two aspartic acids of the human C5aR1 (SEQ ID No.: 2), correspondingly amino acid sequence depicted in SEQ ID No.: 16 covers amino acids 18-26, a fragment of the N-terminus including two aspartic acids of the mouse C5aR1 (SEQ ID No.: 5).

The receptor binding sites in human C5a have been well investigated and have been summarized by Monk et al. (Monk P N et al. Br J Pharmacol. 2007), as following: Human C5a is composed of 74-amino acids, including Asn64, which has an N-linked carbohydrate moiety that is not essential for biological activity but very likely regulates C5a activity in vivo. The solution structure (Zhang X et al. Proteins 1997; Zuiderweg E R and Fesik S W. Biochemistry 1989; Zuiderweg E R et al. Biochemistry 1989) of human C5a has an antiparallel 4-helix bundle (residues 1-63), the four different helical segments (4-12, 18-26, 32-39, 46-63) being stabilized by three disulphide bonds (Cys21-Cys47, Cys22-Cys54, Cys34-Cys55) and connected by loop segments 13-17, 27-33 and 40-45. The 63-residue helix bundle fragment is highly cationic and confers high affinity for the cell surface. The C-terminal residues 69-74 also form a bulky helical turn connected to the 4-helix bundle by a short loop. Reducing disulphide bonds or selectively removing residues before the N-terminal disulphide from C5a 1 to 74 substantially decreases function. The fragment C5a 1-69 missing the C-terminal pentapeptide binds to cells but has no agonist activity, consistent with the N-terminal helix bundle conferring affinity, while the C-terminus alone is the receptor activating domain. Loop 1 (residues C5a 12-20, including four Lys residues 12, 14, 19, 20), loop 3 (C5a39-46) and the C-terminal 6-8 residues (especially Arg74) are important for binding to C5a receptor (C5aR) and agonist potency. Neutralizing antibodies to C5a have implicated the region Lys20-Arg37 as important for receptor binding.

In one embodiment, the binder that is subject matter of the present invention may bind to said region Lys20-Arg37 of C5a.

Regarding C5aR2/C5L2, studies have shown (similar to C5aR1/CD88) that the extracellular N-terminus, containing sulfated Tyr residues flanked by acidic amino acids, plays an important role in ligand binding. Furthermore, both receptors—C5aR1/CD88 and C5aR2/C5L2—are similar in charged and hydrophobic residues in their extracellular and transmembrane domains, suggesting an analogous ligand binding mode (Farzan M et al. J Exp Med. 2001, Okinaga S. et al. Biochemistry 2003, Gao H et al. FASEB J. 2005, Scola A M. J Biol Chem. 2007). C5L2 is able to bind C3a and C4a distinct from the binding site of C5a with a similar affinity as C3aR1, thereby C5L2 can simultaneously bind different complement-anaphylatoxins (Cain S A. et al. J Biol Chem. 2002, Kalant D. et al. J Biol Chem 2003).

Amino acid sequence depicted in SEQ ID No.: 7 covers amino acids 46-59, a fragment of transmembrane domain 1 of the human C5aR2 (SEQ ID No.: 1), which has a 79% identity of corresponding amino acids 48-61, containing Gly51, Asn55 and Val58 that are attributed to play an important role in receptor/ligand binding (Monk P N et al. Br J Pharmacol. 2007), of the human C5aR1 (SEQ ID No.: 2).

Amino acid sequence depicted in SEQ ID No.: 8 covers amino acids 79-88, a fragment of transmembrane domain 2 of the human C5aR2 (SEQ ID No.: 1), which has a 70% identity of corresponding amino acids 81-90, containing Ala81, Asp82, Cys83, Leu85, Leu87 and Pro90 that are attributed to play an important role in receptor/ligand binding (Monk P N et al. Br J Pharmacol. 2007), of the human C5aR1 (SEQ ID No.: 2), and which has a 100% identity of corresponding amino acids 67-76, containing Asp68 that is attributed to play an important role in receptor/ligand binding (Sun J. et al. Protein Sci. 1999) of the human C3aR1 (SEQ ID No.: 3).

Amino acid sequence depicted in SEQ ID No.: 9 covers amino acids 118-126, a fragment of transmembrane domain 3 of the human C5aR2 (SEQ ID No.: 1), which has a 89% identity of corresponding amino acids 120-128, containing Ser123 and Leu126 that are attributed to play an important role in receptor/ligand binding (Monk P N et al. Br J Pharmacol. 2007), of the human C5aR1 (SEQ ID No.: 2).

Amino acid sequence depicted in SEQ ID No.: 10 covers amino acids 161-169, a fragment of transmembrane domain 4 of the human C5aR2 (SEQ ID No.: 1), which has a 89% identity of corresponding amino acids 163-171, containing Leu166, Thr168, Val169, Pro170 and Ser171 that are attributed to play an important role in receptor/ligand binding (Monk P N et al. Br J Pharmacol. 2007), of the human C5aR1 (SEQ ID No.: 2).

Amino acid sequence depicted in SEQ ID No.: 11 covers amino acids 242-249, a fragment of transmembrane domain 6 of the human C5aR2 (SEQ ID No.: 1), which has a 63% identity of corresponding amino acids 251-258, containing Phe251 that is attributed to play an important role in receptor/ligand binding (Monk P N et al. Br J Pharmacol. 2007), of the human C5aR1 (SEQ ID No.: 2), and which has a 75% identity of corresponding amino acids 386-393, adjacent to His394 that is attributed to play an important role in receptor/ligand binding (Sun J. et al. Protein Sci. 1999), of the human C3aR1 (SEQ ID No.: 3).

Amino acid sequence depicted in SEQ ID No.: 12 covers amino acids 98-103, a fragment of extracellular loop 1 domain of the human C5aR2 (SEQ ID No.: 1), which has a 67% identity of corresponding amino acids 100-105, containing Trp102, Phe104 and Gly105 that are attributed to play an important role in receptor/ligand binding (Monk P N et al. Br J Pharmacol. 2007), of the human C5aR1 (SEQ ID No.: 2), and which has a 83% identity of corresponding amino acids 86-91, a fragment of extracellular loop 1 domain of the human C3aR1 (SEQ ID No.: 3).

Amino acid sequence depicted in SEQ ID No.: 13 covers amino acids 13-23, a fragment of the extracellular N-terminal domain of the human C5aR2 (SEQ ID No.: 1), which has a 82% identity of corresponding amino acids 33-43 (SEQ ID No.: 14) of the mouse C5aR2 (SEQ ID No.: 4), containing Tyr14 that is critical for receptor/ligand binding (Farzan M et al. J Exp Med. 2001).

The term “specific binding” is defined as a protein-ligand binding affinity with a dissociation constant of 1 mM or less, preferably 100 μM or less, preferably 50 μM or less, preferably 30 μM or less, preferably 20 μM or less, preferably 10 μM or less, preferably 5 μM or less, more preferably 1 μM or less, more preferably 900 nM or less, more preferably 800 nM or less, more preferably 700 nM or less, more preferably 600 nM or less, more preferably 500 nM or less, more preferably 400 nM or less, more preferably 300 nM or less, more preferably 200 nM or less, even more preferably 100 nM or less, even more preferably 90 nM or less, even more preferably 80 nM or less, even more preferably 70 nM or less, even more preferably 60 nM or less, even more preferably 50 nM or less, even more preferably 40 nM or less, even more preferably 30 nM or less, even more preferably 20 nM or less, and even more preferably 10 nM or less; determined by a radioligand binding assay (Cain S A, Monk P N, J Biol Chem. 2002) or surface plasmon resonance (BIAcore) (Colley C S et al. MAbs. 2018; as described in US 2012/0065254) or ELISA-based binding assay (Michelfelder S., J Am Soc Nephrol. 2018). The radioligand binding assay may be a Radiolabeled Ligand Competition Receptor Binding Assay as described in Kalant et al. J Biol Chem 2003, wherein said Radiolabeled Ligand Competition Receptor Binding Assay determines binding affinities between the complement receptors C5aR1 (also called CD88 in Kalant et al. J Biol Chem 2003), C3aR or C5L2 (SEQ ID No: 1, 2 and 3 of the present invention) and the anaphylatoxins C3a, C4a or C5a in a cell culture system. In said assay, receptor-bound and radiolabeled C3a, C4a or C5a was competitively displaced using increasing concentrations of unlabeled C3a, C4a or C5a. It is known to the person skilled in the art that unlabeled compounds different from of unlabeled C3a, C4a or C5a may be tested for displacement of receptor-bound radiolabeled C3a, C4a or C5a, comprising the use of the binders of the present invention.

The term “inhibiting the activity”, with regard to a protein or protein fragment/peptide, a non-IgG scaffold, an aptamer, oligonucleotides, an antibody or antibody-like proteins, peptidomimetics, or a fragment thereof according to the present invention, refers to the characteristic of inhibiting the process of fibroblast/myofibroblast activation and/or transdifferentiation in the presence of C5a and/or C3a and/or C4a stimulation. For this purpose, fibroblasts (e.g. human corneal keratocytes) incubated for 24 hours with C3a and/or C4a and/or C5a at a concentration of 0.1 μg/ml in DMEM (Dulbecco's Modified Eagle Medium) growth medium without fetal bovine serum (‘stimulation control’) are being compared to fibroblasts, which are incubated under the same conditions but with the addition of a protein or protein fragment/peptide, a non-IgG scaffold, an aptamer, oligonucleotides, an antibody or antibody-like proteins, peptidomimetics, or a fragment thereof according to the present invention that shall be tested for its efficacy (‘inhibition control’). After stimulation, the proportion (given in percentages) of myofibroblasts in a monolayered fibroblast cell culture is being determined by alpha smooth muscle actin (aSMA) immunocytochemistry staining, using anti-aSMA antibodies. Hereby myofibroblasts become apparent as cells that stain positive for aSMA in the cytoplasma. A protein or protein fragment/peptide, a non-IgG scaffold, an aptamer, an antibody or a fragment thereof, according to the present invention, is defined as effective, considering its optimal conditions and concentration, by the means of “inhibiting the activity” of myofibroblast activation if the proportion of myofibroblasts in the ‘inhibition control’ can be reduced preferably by at least 10%, more preferably by at least 20%, even more preferably by at least 25%, even more preferably by at least 30%, even more preferably by at least 35%, even more preferably by at least 40%, even more preferably by at least 45%, even more preferably by at least 50%, even more preferably by at least 55%, even more preferably by at least 60%, and even more preferably by at least 65%, compared to the proportion of myofibroblasts in the ‘stimulation control’.

In one embodiment of the invention said binder is a protein or protein fragment is selected from the group comprising human C5L2 protein according to SEQ ID No.: 1, a protein or fragment that is at least 60% identical to the full-length amino acid sequence of human C5L2 protein of SEQ ID No.:1, human C5aR1 protein according to SEQ ID No.: 2, a protein or fragment that is at least 60% identical to the full-length amino acid sequence of human C5aR1 protein of SEQ ID No.: 2, human C3aR protein according to SEQ ID No.: 3, a protein or fragment that is at least 60% identical to the full-length amino acid sequence of human C3aR protein of SEQ ID No.: 3, mouse C5L2 protein according to SEQ ID No.: 4, a protein or fragment that is at least 60% identical to the full-length amino acid sequence of mouse C5L2 protein of SEQ ID No.:4, mouse C5aR1 protein according to SEQ ID No.: 5, a protein or fragment that is at least 60% identical to the full-length amino acid sequence of mouse C5aR1 protein of SEQ ID No.: 5, mouse C3aR protein according to SEQ ID No.: 6, and a protein or fragment that is at least 60% identical to the full-length amino acid sequence of mouse C3aR protein of SEQ ID No.: 6.

In a specific embodiment of the invention the identity to the respective full-length amino acid sequence is least 65%, or at least 70%, or at least 75%, or at least 80%, or at least 85%, or at least 90%, or at least 95%, or at least 97%, or at least 98%, or at least 99%.

In one embodiment, full-length recombinant human C5a anaphylatoxin chemotactic receptor 2 (rhC5AR2/rhC5L2) may be produced in wheat germ (ab153291; Abcam; Cambridge, UK).

In another embodiment, full-length recombinant human C5a anaphylatoxin chemotactic receptor 1 (rhC5AR1) located on the cell membrane may produced in wheat germ (ab157989; Abcam; Cambridge, UK) and post-translationally modified by sulfation.

In another embodiment, full-length recombinant human C3a anaphylatoxin chemotactic receptor (rhC3AR) located on the cell membrane may be produced in wheat germ (ab152249; Abcam; Cambridge, UK), and sulfated on Tyr174.

The extent of a identity between two amino acid sequences is defined as the result of heuristic algorithms, such as FASTA (Lipman D J et al. Science 1985, Pearson W R et al. PNAS 1988) and basic local alignment search tool (BLAST). (Lobo I. Nature Education 2008). The identity of a protein/peptide or protein fragment that shall be tested, to an amino acid sequence according to SEQ ID No's.: 1-17, is 100% if the protein/peptide or protein fragment that is tested is identical (respectively has a BLAST result of 100% identity) or contains a fragment identical (respectively has a BLAST result of 100% identity) to SEQ ID No's.: 1-17.

In one embodiment of the invention said protein or protein fragment comprises at least one conserved region selected from the group comprising an amino acid sequence according to SEQ ID No.:7, an amino acid sequence according to SEQ ID No.:8, an amino acid sequence according to SEQ ID No.:9, an amino acid sequence according to SEQ ID No.:10, an amino acid sequence according to SEQ ID No.:11, an amino acid sequence according to SEQ ID No.:12, an amino acid sequence according to SEQ ID No.:13, an amino acid sequence according to SEQ ID No.:14, an amino acid sequence according to SEQ ID No.:15, an amino acid sequence according to SEQ ID No.:16, an amino acid sequence according to SEQ ID No.:17, and a protein or fragment that is at least 60% identical to any of the amino acid sequences according to SEQ ID No's.:7-17.

In one embodiment of the invention said protein or protein fragment comprises at least two conserved regions selected from the group comprising an amino acid sequence according to SEQ ID No.:7, an amino acid sequence according to SEQ ID No.:8, an amino acid sequence according to SEQ ID No.:9, an amino acid sequence according to SEQ ID No.:10, an amino acid sequence according to SEQ ID No.:11, an amino acid sequence according to SEQ ID No.:12, an amino acid sequence according to SEQ ID No.:13, an amino acid sequence according to SEQ ID No.:14, an amino acid sequence according to SEQ ID No.:15, an amino acid sequence according to SEQ ID No.:16, an amino acid sequence according to SEQ ID No.:17, and a protein or fragment that is at least 60% identical to any of the amino acid sequences according to SEQ ID No's.:7-17.

In another embodiment of the invention said protein or protein fragment comprises at least three of the before-mentioned conserved regions, or at least four of the before-mentioned conserved regions, or at least five of the before-mentioned conserved regions, or six of the before-mentioned conserved regions.

In one embodiment of the invention the conserved regions exhibit at least at least 65%, or at least 75%, or at least 80%, or at least 85%, or at least 90%, or at least 95%, or at least 97%, or at least 98%, or at least 99% sequence identity to any of the before-mentioned amino acids according to SEQ ID No.: 7-17.

Table 1 provides an overview of sequence identities, determined by BLAST, between corresponding amino acid sequences of conserved sequence fragments (SEQ ID No's.: 7-17) characteristic for human and mouse C5L2, C5AR1 and C3AR.

TABLE 1
Identities of corresponding amino acid
sequences corresponding.

| | | human | mouse | human | mouse | human | mouse |
|---|---|---|---|---|---|---|---|
| SEQ ID | Sequence | C5L2 | C5L2 | C5AR1 | C5AR1 | C3AR | C3AR |
| No.: 7 | FLVGVPGNAMVAWV | 100% | 86% | 79% | 86% | 64% | 50% |
| No.: 8 | ADLLCCLSLP | 100% | 90% | 70% | 80% | 100% | 90% |
| No.: 9 | MYASVLLLA | 100% | 89% | 89% | 89% | 67% | 67% |
| No.: 10 | LALLLTVPS | 100% | 89% | 89% | 89% | 22% | 33% |
| No.: 11 | FFVCWAPY | 100% | 75% | 63% | 63% | 75% | 75% |
| No.: 12 | GHWPYG | 100% | 100% | 67% | 17% | 83% | 100% |
| No.: 13 | YSDLSDRPVDC | 100% | 82% | 45% | 18% | 0% | 36% |
| No.: 14 | YSDLPDVPVDC | 82% | 100% | 45% | 36% | 0% | 27% |
| No.: 15 | TLDLNTPVD | 33% | 56% | 100% | 56% | 0% | 33% |
| No.: 16 | TMDPNIPAD | 22% | 44% | 56% | 100% | 0% | 11% |
| No.: 17 | PLVAITITRL | 30% | 0% | 0% | 40% | 100% | 90% |

Bold = Identity to the corresponding amino acid sequences is >60%.

Subject matter of the present invention is a composition comprising at least one binder, e.g. a proteins or protein fragment, according to the present invention for use in the treatment of a subject having an ocular wound and/or fibrosis.

Subject matter of the present invention is a composition comprising at least two binders, e.g. two proteins/peptides or protein fragments, according to the present invention for use in the treatment of a subject having an ocular wound and/or fibrosis.

Subject matter of the present invention is a composition comprising at least three binders, e.g. proteins/peptides or protein fragments, according to the present inventions for use in the treatment of a subject having an ocular wound and/or fibrosis. For the purpose of clarity, it is herein understood that the word “fibrosis” within the wording “ocular wound and/or fibrosis” refers to the general definition of the term “fibrosis” and is not limited to ocular fibrosis only, wherein the wording “ocular wound and/or fibrosis” and “fibrosis and/or ocular wound” can be used interchangeably herein.

One binder, e.g. protein or protein fragment, may contain one or multiple binding sites for C5a and/or C3a and/or C4a. According to Table 1, the number of binding sites may vary, depending on the number of comprised sequences selected from SEQ ID No.: 1-17.

In this regard, a composition of more than one binder, e.g. protein/peptide or protein fragment comprising SED ID No.: 1-17 expands the inhibiting effect on C3a-, C4a- and C5a-dependent activities. In particular, the combination of proteins or protein fragments deriving from primarily C3a-binding moieties, such as SEQ ID No's: 8, 12 and 17, with proteins or protein fragments deriving from primarily C5a-binding moieties, such as SEQ ID No's: 7, 9, 10, 11, 13, 14, 15 and 16, are of particular importance.

Subject matter of the present invention is a pharmaceutical composition comprising a binder, e.g. protein or protein fragment, according to the present invention or a composition according to the present invention for use in the treatment of a subject having an ocular wound and/or fibrosis.

The binders of the present invention may be pegylated, or altered in a comparable way, to modify the biological stability and/or half-life of the binder. PEGylation is the process of both covalent and non-covalent attachment or amalgamation of polyethylene glycol (PEG, in pharmacy called macrogol) polymer chains to molecules and macrostructures, such as a drug, therapeutic protein or vesicle, which is then described as PEGylated (pegylated). PEGylation is routinely achieved by the incubation of a reactive derivative of PEG with the target molecule. The covalent attachment of PEG to a drug or therapeutic protein can “mask” the agent from the host's immune system (reducing immunogenicity and antigenicity), and increase its hydrodynamic size (size in solution), which prolongs its circulatory time by reducing renal clearance.

The binders of the present invention may undergo posttranslational or post-synthesis modifications that may comprise i.a. the attachment of sugars, fatty acids, phosphate groups (phosphoryl group, phosphorylation), hydroxyl groups, methyl groups (methylation of proteins), ubiquitin (ubiquitination of proteins), to alter the actual structure of the binder and may enhance its function or stability. These modification may be made on both, the amino (amino terminus) and carboxyl end (carboxyl terminus) of a binder, as well as amino acid side chains (amino acids) within the protein and may be reversible and/or irreversible.

Subject matter are furthermore prodrugs of the binder according to the present invention. A prodrug is a medication or compound that, after administration, is metabolized (i.e., converted within the body) into a pharmacologically active drug. Inactive prodrugs are pharmacologically inactive medications that are metabolized into an active form within the body. Instead of administering a drug directly, a corresponding prodrug might be used instead to improve how a medicine is absorbed, distributed, metabolized, and excreted.

In one embodiment of the invention said pharmaceutical composition is for topical application, i.e. is topically administered.

In one embodiment of the invention said pharmaceutical composition is for intraocular application, i.e. is intraocular administered.

In one embodiment of the invention said pharmaceutical composition is for intravitreal application, i.e. is intravitreal administered.

In one embodiment of the invention said pharmaceutical composition is for subconjunctival application, i.e. is subconjunctival administered.

In one embodiment of the invention said pharmaceutical composition is for intravascular/intravenous application, i.e. is intravascular/intravenous administered.

One embodiment of the present invention is a binder, e.g. protein or protein fragment, according to the present invention or a composition according to the present invention or a pharmaceutical composition according to the present invention for use in the treatment of a subject wherein said subject suffers from a disease selected from the group comprising: conjunctivitis and conjunctival scars (including ocular pemphigoid), scleritis and episcleritis, corneal scars and opacities due to corneal ulcer, keratoconjunctivitis, keratitis, bullous keratopathy, corneal degenerations, iridocyclitis and adhesions of iris and ciliary body, chorioretinal scars/fibrosis due to chorioretinal inflammation or degeneration or haemorrhage or rupture or neovascularization, fibrotic vitreoretinopathies, such as in proliferative vitreoretinopathy, retinopathy of prematurity and diabetic retinopathy; choroidal neovascularization and degenerations of the macula, secondary glaucoma, endophthalmitis, and impairments of wound healing and fibrosis after ocular surgery or trauma, including intraocular foreign bodies.

One embodiment of the present invention is a binder, e.g. protein/peptide or protein fragment, according to the present invention or a composition according to the present invention or a pharmaceutical composition according to the present invention for use in the treatment of a subject wherein said subject suffers from corneal fibrosis.

One embodiment of the present invention is a binder, e.g. protein/peptide or protein fragment, according to the present invention or a composition according to the present invention or a pharmaceutical composition according to the present invention for use in the treatment of a subject wherein said subject suffers from chorioretinal fibrosis.

One embodiment of the present invention is a binder, e.g. protein/peptide or protein fragment, according to the present invention or a composition according to the present invention or a pharmaceutical composition according to the present invention for use in the treatment of a subject wherein said subject suffers from impairments of wound healing and fibrosis after ocular surgery or trauma.

One embodiment of the present invention is binder, e.g. protein or protein fragment, according to the present invention or a composition according to the present invention or a pharmaceutical composition according to the present invention for use in the treatment of a subject wherein said subject suffers from a disease selected from the group comprising: (idiopathic) pulmonary fibrosis, dermal keloid formation, scleroderma, myelofibrosis, kidney-, pancreas- and heart-fibrosis, and fibrosis in (non)-alcoholic steatohepatosis, glomerulonephritis and (ANCA-associated) vasculitis.

One embodiment of the present invention is a binder, e.g. protein or protein fragment, according to the present invention or a composition according to the present invention or a pharmaceutical composition according to the present invention for use in the treatment of a subject wherein said subject suffers from pulmonary fibrosis.

One embodiment of the present invention is a binder, e.g. protein or protein fragment, according to the present invention or a composition according to the present invention or a pharmaceutical composition according to the present invention for use in the treatment of a subject wherein said subject suffers from fibrosis due to glomerulonephritis and/or renal fibrosis

One embodiment of the present invention is a binder, e.g. protein or protein fragment, according to the present invention or a composition according to the present invention or a pharmaceutical composition according to the present invention for use in the treatment of a subject wherein said subject suffers from steatohepatosis and/or liver fibrosis.

The following embodiments are subject of the invention:
1. Binder binding to complement-anaphylatoxin C5a and/or C3a and/or C4a and thereby preferably inhibiting the activity of C5a and/or C3a and/or C4a for use in the treatment of a subject having an ocular wound and/or fibrosis.
2. Binder for use in the treatment of a subject having an ocular wound and/or fibrosis according to embodiment 1 wherein said binder is selected from the group comprising a protein or a fragment thereof, a peptide, a non-IgG scaffold, an aptamer, oligonucleotides, an antibody or antibody-like proteins, peptidomimetics or a fragment thereof.
3. Binder for use in the treatment of a subject having an ocular wound and/or fibrosis according to embodiment 1 or 2 wherein said binder is a protein or a fragment thereof.
4. Binder according to any of embodiments 1 to 3 for use in the treatment of a subject having an ocular wound and/or fibrosis wherein said binder is administered to promote wound healing, in particular corneal wound healing.
5. Binder according to any of embodiments 1 to 4 for use in the treatment of a subject having an ocular wound and/or fibrosis wherein said binder binds to C5a and C3a and thereby essentially inhibiting the activity of C5a and C3a.
6. Binder according to any of embodiments 1 to 5 for use in the treatment of a subject having an ocular wound and/or fibrosis wherein said binder binds to C5a and C4a and thereby essentially inhibiting the activity of C5a and C4a.
7. Binder according to any of embodiments 1 to 6 for use in the treatment of a subject having an ocular wound and/or fibrosis wherein said binder binds to C3a and C4a and thereby essentially inhibiting the activity of C3a and C4a.
8. Binder according to any of embodiments 1 to 7 for use in the treatment of a subject having an ocular wound and/or fibrosis wherein said binder binds to C5a and C3a and C4a and thereby inhibiting the activity of C5a and C3a and C4a.
9. Binder according to any of embodiments 1 to 8 for use in the treatment of a subject having an ocular wound and/or fibrosis wherein said binder is a protein or protein fragment is selected from the group comprising human C5L2 protein according to SEQ ID No.: 1, a protein/peptide or fragment that is at least 60% identical to the full-length amino acid sequence of human C5L2 protein of SEQ ID No.:1, human C5aR1 protein according to SEQ ID No.: 2, a protein or fragment that is at least 60% identical to the full-length amino acid sequence of human C5aR1 protein of SEQ ID No.: 2, human C3aR protein according to SEQ ID No.: 3, a protein or fragment that is at least 60% identical to the full-length amino acid sequence of human C3aR protein as of SEQ ID No.: 3, a mouse C5L2 protein according to SEQ ID No.: 4, a protein or fragment that is at least 60% identical to the full-length amino acid sequence of mouse C5L2 protein of SEQ ID No.:4, mouse C5aR1 protein according to SEQ ID No.: 5, a protein or fragment that is at least 60% identical to the full-length amino acid sequence of mouse C5aR1 protein of SEQ ID No.: 5, mouse C3aR protein according to SEQ ID No.: 6, and a protein or fragment that is at least 60% identical to the full-length amino acid sequence of mouse C3aR protein of SEQ ID No.: 6.
10. Binder according to any of embodiments 1 to 9 for use in the treatment of a subject having an ocular wound and/or fibrosis wherein said binder is a protein/peptide or protein fragment and comprises at least one conserved region selected from the group comprising an amino acid sequence according to SEQ ID No.:7, an amino acid sequence according to SEQ ID No.:8, an amino acid sequence according to SEQ ID No.:9, an amino acid sequence according to SEQ ID No.:10, an amino acid sequence according to SEQ ID No.:11, an amino acid sequence according to SEQ ID No.:12, an amino acid sequence according to SEQ ID No.:13, an amino acid sequence according to SEQ ID No.:14, an amino acid sequence according to SEQ ID No.:15, an amino acid sequence according to SEQ ID No.:16, an amino acid sequence according to SEQ ID No.:17, and a protein or fragment that is at least 60% identical to any of the amino acid sequences according to SEQ ID No's.:7-17.
11. Binder according to embodiments 10 for use in the treatment of a subject having an ocular wound and/or fibrosis wherein said binder is a protein/peptide or protein fragment and comprises at least two conserved region selected from the group comprising an amino acid sequence according to SEQ ID No.:7, an amino acid sequence according to SEQ ID No.:8, an amino acid sequence according to SEQ ID No.:9, an amino acid sequence according to SEQ ID No.:10, an amino acid sequence according to SEQ ID No.:11, an amino acid sequence according to SEQ ID No.:12, an amino acid sequence according to SEQ ID No.:13, an amino acid sequence according to SEQ ID No.:14, an amino acid sequence according to SEQ ID No.:15, an amino acid sequence according to SEQ ID No.:16, an amino acid sequence according to SEQ ID No.:17, and a protein or fragment that is at least 60% identical to any of the amino acid sequences according to SEQ ID No's.:7-17.
12. Composition comprising at least two binders, preferably proteins or protein fragments, according to any of embodiments 1 to 11 for use in the treatment of a subject having an ocular wound and/or fibrosis.
13. Composition comprising at least three proteins or protein fragments according to any of embodiments 1 to 9 for use in the treatment of a subject having an ocular wound and/or fibrosis.
14. Pharmaceutical composition comprising a binder according to any of embodiments 1-11 or a composition according to embodiments 12 or 13 for use in the treatment of a subject having an ocular wound and/or fibrosis.
15. Pharmaceutical composition according embodiments 14 wherein said pharmaceutical composition further comprises a carrier and/or an excipient and/or a stabilizer.
16. Pharmaceutical composition according embodiments 14 or 15 for topical application.
17. Pharmaceutical composition according embodiments 14 or 15 for intraocular application.
18. Pharmaceutical composition according embodiments 14 or 15 for intravitreal application.
19. Pharmaceutical composition according embodiments 14 or 15 for subconjunctival application.
20. Binder according to any of embodiments 1-11 or a composition according to embodiments 12 or 13 or a pharmaceutical composition of any of embodiments 14-19 for use in the treatment of a subject wherein said subject suffers from a disease selected from the group comprising: conjunctivitis and conjunctival scars (including ocular pemphigoid), scleritis and episcleritis, corneal scars and opacities due to corneal ulcer, keratoconjunctivitis, keratitis, bullous keratopathy, corneal degenerations, iridocyclitis and adhesions of iris and ciliary body, chorioretinal scars/fibrosis due to chorioretinal inflammation or degeneration or haemorrhage or rupture or neovascularization, fibrotic vitreoretinopathies, such as in proliferative vitreoretinopathy, retinopathy of prematurity and diabetic retinopathy; choroidal neovascularization and degenerations of the macula, secondary glaucoma, endophthalmitis, and impairments of wound healing and fibrosis after ocular surgery or trauma, including intraocular foreign bodies.
21. Binder according to any of embodiments 1-11 or a composition according to embodiments 12 or 13 or a pharmaceutical composition of any of embodiments 14-19 for use in the treatment of a subject wherein said subject suffers from a disease selected from the group comprising: (idiopathic) pulmonary fibrosis, dermal keloid formation, scleroderma, myelofibrosis, kidney-, pancreas- and heart-fibrosis, and fibrosis in (non)-alcoholic steatohepatosis, glomerulonephritis and (ANCA-associated) vasculitis.

The following embodiments are subject of the invention:
1. Binder binding to complement-anaphylatoxin C5a and/or C3a and/or C4a and thereby preferably inhibiting the activity of C5a and/or C3a and/or C4a for use in the treatment of a subject having an ocular wound and/or fibrosis.
2. Binder for use in the treatment of a subject having an ocular wound and/or fibrosis according to claim 1 wherein said binder is selected from the group comprising a protein or a fragment thereof, a peptide, a non-IgG scaffold, an aptamer, oligonucleotides, an antibody or antibody-like proteins, peptidomimetics or a fragment thereof.
3. Binder for use in the treatment of a subject having an ocular wound and/or fibrosis according to claim 1 or 2 wherein said binder is a protein or a fragment thereof.
4. Binder according to any of claims 1 to 3 for use in the treatment of a subject having an ocular wound and/or fibrosis wherein said binder is administered to promote wound healing, in particular corneal wound healing.
5. Binder according to any of claims 1 to 4 for use in the treatment of a subject having an ocular wound and/or fibrosis wherein said binder binds to C5a and C3a and thereby essentially inhibiting the activity of C5a and C3a.
6. Binder according to any of claims 1 to 5 for use in the treatment of a subject having an ocular wound and/or fibrosis wherein said binder binds to C5a and C4a and thereby essentially inhibiting the activity of C5a and C4a.
7. Binder according to any of claims 1 to 6 for use in the treatment of a subject having an ocular wound and/or fibrosis wherein said binder binds to C3a and C4a and thereby essentially inhibiting the activity of C3a and C4a.
8. Binder according to any of claims 1 to 7 for use in the treatment of a subject having an ocular wound and/or fibrosis wherein said binder binds to C5a and C3a and C4a and thereby inhibiting the activity of C5a and C3a and C4a.
9. Binder according to any of claims 1-6 or 8 for use in the treatment of a subject having an ocular wound and/or fibrosis, wherein said binder may bind to several overlapping peptide fragments of a complement component C5a protein having the amino acid sequence depicted in SEQ ID No.: 20 or SEQ ID No.: 21, wherein overlapping means the overlapping of the targeted amino acid sequences of the antibody, antibody-like protein or binder and the specific peptide fragments.
10. Binder according to claim 9 for use in the treatment of a subject having an ocular wound and/or fibrosis, wherein said binder may bind only to C5a at an epitope within or overlapping with a fragment of the protein having the amino acid sequence, according to SEQ ID No's.: 22-34.
11. Binder according to claim 9 for use in the treatment of a subject having an ocular wound and/or fibrosis, wherein said binder may also bind to an epitope of C5a formed by amino acid sequences according to SEQ ID No's: 35-40 (SEQ ID No.: 35: X₁X₂ETCEX₃RX₄, SEQ ID No.: 36: X₅X₆KX₇X₈X₉L and SEQ ID No.: 37: X₅X₆KX₇X₈X₉I), wherein X₁ is selected from the group consisting of N, H, D, F, K, Y, and T; X₂ is selected from the group consisting of D, L, Y, and H; X₃ is selected from the group consisting of Q, E, and K; X₄ is selected from the group consisting of A, V, and L; X₅ is selected from the group consisting of S, H, P, and N; X₆ is selected from the group consisting of H and N; X₇ is selected from the group consisting of D, N, H, P, and G; X₈ is selected from the group consisting of M, L, I, and V; and X₉ is selected from the group consisting of Q, L, and I.
12. Binder according to any of claims 1-5, 7 or 8 for use in the treatment of a subject having an ocular wound and/or fibrosis, wherein said binder may bind to several overlapping peptide fragments of a complement component C3a protein having the amino acid sequence depicted in SEQ ID No.: 43.
13. Binder according to claim 12 for use in the treatment of a subject having an ocular wound and/or fibrosis, wherein said binder may also bind only to a human C3a at an epitope within or overlapping with a fragment of the protein having the amino acid sequence, according to SEQ ID No's.: 44-47.
14. Binder according to any of claims 1-4 or 6-8 for use in the treatment of a subject having an ocular wound and/or fibrosis, wherein said binder may bind to several overlapping peptide fragments of a complement component C4a protein having the amino acid sequence depicted in SEQ ID No.: 48 or SEQ ID No.: 49.
15. Binder according to claim 13 for use in the treatment of a subject having an ocular wound and/or fibrosis, wherein said binder may also bind only to a human C4a at an epitope within or overlapping with a fragment of the protein having the amino acid sequence, according to SEQ ID No.: 50.
16. Binder according to claims 1-15 for use in the treatment of a subject having an ocular wound and/or fibrosis, wherein said binder is an antibody or an antibody-like protein.
17. Binder according to claims 1-15 for use in the treatment of a subject having an ocular wound and/or fibrosis, wherein said binder is an aptamer.
18. Binder according to claim 17 for use in the treatment of a subject having an ocular wound and/or fibrosis, wherein said binder is an aptamer, and wherein said aptamer may relate to a nucleic acid molecule consisting of RNA and/or DNA, such as disclosed in SEQ ID No.: 41.
19. Binder according to claim 18 for use in the treatment of a subject having an ocular wound and/or fibrosis, wherein said binder is an aptamer, and wherein said aptamer may relate to a nucleic acid molecule consisting of RNA and/or DNA, such as disclosed in SEQ ID No.: 41, and wherein said aptamer binds to a binding site on C5a comprising SEQ ID No: 42.
20. Binder according to any of claims 1 to 19 for use in the treatment of a subject having an ocular wound and/or fibrosis wherein said binder is a protein or protein fragment is selected from the group comprising human C5L2 protein according to SEQ ID No.: 1, a protein/peptide or fragment that is at least 60% identical to the full-length amino acid sequence of human C5L2 protein of SEQ ID No.:1, human C5aR1 protein according to SEQ ID No.: 2, a protein or fragment that is at least 60% identical to the full-length amino acid sequence of human C5aR1 protein of SEQ ID No.: 2, human C3aR protein according to SEQ ID No.: 3, a protein or fragment that is at least 60% identical to the full-length amino acid sequence of human C3aR protein as of SEQ ID No.: 3, a mouse C5L2 protein according to SEQ ID No.: 4, a protein or fragment that is at least 60% identical to the full-length amino acid sequence of mouse C5L2 protein of SEQ ID No.:4, mouse C5aR1 protein according to SEQ ID No.: 5, a protein or fragment that is at least 60% identical to the full-length amino acid sequence of mouse C5aR1 protein of SEQ ID No.: 5, mouse C3aR protein according to SEQ ID No.: 6, and a protein or fragment that is at least 60% identical to the full-length amino acid sequence of mouse C3aR protein of SEQ ID No.: 6.
21. Binder according to any of claims 1 to 20 for use in the treatment of a subject having an ocular wound and/or fibrosis wherein said binder is a protein/peptide or protein fragment and comprises at least one conserved region selected from the group comprising an amino acid sequence according to SEQ ID No.:7, an amino acid sequence according to SEQ ID No.:8, an amino acid sequence according to SEQ ID No.:9, an amino acid sequence according to SEQ ID No.:10, an amino acid sequence according to SEQ ID No.:11, an amino acid sequence according to SEQ ID No.:12, an amino acid sequence according to SEQ ID No.:13, an amino acid sequence according to SEQ ID No.:14, an amino acid sequence according to SEQ ID No.:15, an amino acid sequence according to SEQ ID No.:16, an amino acid sequence according to SEQ ID No.:17, and a protein or fragment that is at least 60% identical to any of the amino acid sequences according to SEQ ID No's.:7-17.
22. Binder according to claim 21 for use in the treatment of a subject having an ocular wound and/or fibrosis wherein said binder is a protein/peptide or protein fragment and comprises at least two conserved region selected from the group comprising an amino acid sequence according to SEQ ID No.:7, an amino acid sequence according to SEQ ID No.:8, an amino acid sequence according to SEQ ID No.:9, an amino acid sequence according to SEQ ID No.:10, an amino acid sequence according to SEQ ID No.:11, an amino acid sequence according to SEQ ID No.:12, an amino acid sequence according to SEQ ID No.:13, an amino acid sequence according to SEQ ID No.:14, an amino acid sequence according to SEQ ID No.:15, an amino acid sequence according to SEQ ID No.:16, an amino acid sequence according to SEQ ID No.:17, and a protein or fragment that is at least 60% identical to any of the amino acid sequences according to SEQ ID No's.:7-17.
23. Binder according to any of claims 1-22, for use in the treatment of a subject having an ocular wound and/or fibrosis, wherein inhibition of C3a and/or C4a and/or C5a via said binder may be determined by a cellular activation assay, preferably a fibroblast/myofibroblast activation and/or transdifferentiation assay.
24. Binder according to any of claims 1-23, for use in the treatment of a subject having an ocular wound and/or fibrosis, wherein inhibition of C3a and/or C4a and/or C5a via said binder may be determined by a cellular activation assay, preferably a fibroblast/myofibroblast activation and/or transdifferentiation assay, and wherein said binder selected from the group comprising protein or protein fragment/peptide, a non-IgG scaffold, an aptamer, an antibody or a fragment thereof is effective by means of inhibiting the activity of myofibroblast activation preferably by at least 10%, more preferably by at least 20%, even more preferably by at least 25%, even more preferably by at least 30%, even more preferably by at least 35%, even more preferably by at least 40%, even more preferably by at least 45%, even more preferably by at least 50%, even more preferably by at least 55%, even more preferably by at least 60%, and even more preferably by at least 65%.
25. Binder according to claims 1-24, for use in the treatment of a subject having an ocular wound and/or fibrosis, wherein said binder at least essentially inhibits the process of fibroblast/myofibroblast activation and/or transdifferentiation and has preferably a molecular weight less than 90 kDa, preferably less than 80 kDa or less, preferably less than 70 kDa or less, more preferably less than 60 kDa or less, more preferably less than 50 kDa or less, more preferably less than 45 kDa or less, more preferably less than 40 kDa or less, even more preferably less than 35 kDa or less, even more preferably less than 30 kDa or less, even more preferably less than 25 kDa or less, even more preferably less than 20 kDa or less, even more preferably less than 15 kDa or less, and even more preferably less than 10 kDa or less.
26. Composition comprising at least two binders, preferably proteins or protein fragments, according to any of claims 1 to 25 for use in the treatment of a subject having an ocular wound and/or fibrosis.
27. Composition comprising at least three proteins or protein fragments according to any of claims 1 to 26 for use in the treatment of a subject having an ocular wound and/or fibrosis.
28. Pharmaceutical composition comprising a binder according to any of claims 1-25 or a composition according to claims 26 or 27 for use in the treatment of a subject having an ocular wound and/or fibrosis.
29. Binder according to any of claims 1-25 or a composition according to claims 25 or 26 or a pharmaceutical composition of claim 28 for use in the treatment of a subject wherein said subject suffers from a disease selected from the group comprising: conjunctivitis and conjunctival scars (including ocular pemphigoid), scleritis and episcleritis, corneal scars and opacities due to corneal ulcer, keratoconjunctivitis, keratitis, bullous keratopathy, corneal degenerations, iridocyclitis and adhesions of iris and ciliary body, chorioretinal scars/fibrosis due to chorioretinal inflammation or degeneration or haemorrhage or rupture or neovascularization, fibrotic vitreoretinopathies, such as in proliferative vitreoretinopathy, retinopathy of prematurity and diabetic retinopathy; choroidal neovascularization and degenerations of the macula, secondary glaucoma, endophthalmitis, and impairments of wound healing and fibrosis after ocular surgery or trauma, including intraocular foreign bodies.
30. Binder according to any of claims 1-25 or a composition according to claims 26 or 27 or a pharmaceutical composition of claim 28 for use in the treatment of a subject wherein said subject suffers from a disease selected from the group comprising: (idiopathic) pulmonary fibrosis, dermal keloid formation, scleroderma, myelofibrosis, kidney-, pancreas- and heart-fibrosis, and fibrosis in (non)-alcoholic steatohepatosis, glomerulonephritis and (ANCA-associated) vasculitis.
31. Binder according to any of claims 1-25 or a composition according to claims 26 or 27 or a pharmaceutical composition of claim 28 for use in the treatment of a subject wherein said subject suffers from pulmonary fibrosis.
32. Binder according to any of claims 1-25 or a composition according to claims 26 or 27 or a pharmaceutical composition of claim 28 for use in the treatment of a subject wherein said subject suffers from corneal fibrosis.
33. Binder according to any of claims 1-25 or a composition according to claims 26 or 27 or a pharmaceutical composition of claim 28 for use in the treatment of a subject wherein said subject suffers from chorioretinal fibrosis.
34. Binder according to any of claims 1-25 or a composition according to claims 26 or 27 or a pharmaceutical composition of claim 28 for use in the treatment of a subject wherein said subject suffers from fibrosis due to glomerulonephritis and/or renal fibrosis.
35. Binder according to any of claims 1-25 or a composition according to claims 26 or 27 or a pharmaceutical composition of claim 28 for use in the treatment of a subject wherein said subject suffers from steatohepatosis and/or liver fibrosis.

The following embodiments are subject of the invention:
1. Binder binding to complement-anaphylatoxin C5a and/or C3a and/or C4a and thereby preferably inhibiting the activity of C5a and/or C3a and/or C4a for use in the treatment of a subject having an ocular wound and/or fibrosis.
2. Binder for use in the treatment of a subject having an ocular wound and/or fibrosis according to claim 1 wherein said binder is selected from the group comprising a protein or a fragment thereof, a peptide, a non-IgG scaffold, an aptamer, oligonucleotides, an antibody or antibody-like proteins, peptidomimetics or a fragment thereof.
3. Binder according to any of claims 1 or 2 for use in the treatment of a subject having an ocular wound and/or fibrosis wherein said binder is administered to promote wound healing, in particular corneal wound healing.
4. Binder according to any of claims 1-3 for use in the treatment of a subject having an ocular wound and/or fibrosis, wherein said binder may bind to several overlapping peptide fragments of a complement component C5a protein having the amino acid sequence depicted in SEQ ID No.: 20 or SEQ ID No.: 21, wherein overlapping means the overlapping of the targeted amino acid sequences of the antibody, antibody-like protein or binder and the specific peptide fragments.
5. Binder according to claim 4 for use in the treatment of a subject having an ocular wound and/or fibrosis, wherein said binder may bind only to C5a at an epitope within or overlapping with a fragment of the protein having the amino acid sequence, according to SEQ ID No's.: 22-34.
6. Binder according to claim 4 for use in the treatment of a subject having an ocular wound and/or fibrosis, wherein said binder may also bind to an epitope of C5a formed by amino acid sequences according to SEQ ID No's: 35-40 (SEQ ID No.: 35: X₁X₂ETCEX₃RX₄, SEQ ID No.: 36: X₅X₆KX₇X₈X₉L and SEQ ID No.: 37: X₅X₆KX₇X₈X₉I), wherein X₁ is selected from the group consisting of N, H, D, F, K, Y, and T; X₂ is selected from the group consisting of D, L, Y, and H; X₃ is selected from the group consisting of Q, E, and K; X₄ is selected from the group consisting of A, V, and L; X₅ is selected from the group consisting of S, H, P, and N; X₆ is selected from the group consisting of H and N; X₇ is selected from the group consisting of D, N, H, P, and G; X₈ is selected from the group consisting of M, L, I, and V; and X₉ is selected from the group consisting of Q, L, and I.
7. Binder according to any of claims 1-3 for use in the treatment of a subject having an ocular wound and/or fibrosis, wherein said binder may bind to several overlapping peptide fragments of a complement component C3a protein having the amino acid sequence depicted in SEQ ID No.: 43.
8. Binder according to claim 7 for use in the treatment of a subject having an ocular wound and/or fibrosis, wherein said binder may also bind only to a human C3a at an epitope within or overlapping with a fragment of the protein having the amino acid sequence, according to SEQ ID No's.: 44-47.
9. Binder according to any of claims 1-3 for use in the treatment of a subject having an ocular wound and/or fibrosis, wherein said binder may bind to several overlapping peptide fragments of a complement component C4a protein having the amino acid sequence depicted in SEQ ID No.: 48 or SEQ ID No.: 49.
10. Binder according to claim 9 for use in the treatment of a subject having an ocular wound and/or fibrosis, wherein said binder may also bind only to a human C4a at an epitope within or overlapping with a fragment of the protein having the amino acid sequence, according to SEQ ID No.: 50.
11. Binder according to claims 1-10 for use in the treatment of a subject having an ocular wound and/or fibrosis, wherein said binder is an antibody or an antibody-like protein.
12. Binder according to claims 1-10 for use in the treatment of a subject having an ocular wound and/or fibrosis, wherein said binder is an aptamer.
13. Binder according to claim 12 for use in the treatment of a subject having an ocular wound and/or fibrosis, wherein said binder is an aptamer, and wherein said aptamer may relate to a nucleic acid molecule consisting of RNA and/or DNA, such as disclosed in SEQ ID No.: 41.
14. Binder according to claim 13 for use in the treatment of a subject having an ocular wound and/or fibrosis, wherein said binder is an aptamer, and wherein said aptamer may relate to a nucleic acid molecule consisting of RNA and/or DNA, such as disclosed in SEQ ID No.: 41, and wherein said aptamer binds to a binding site on C5a comprising SEQ ID No: 42.
15. Binder according to any of claims 1 to 14 for use in the treatment of a subject having an ocular wound and/or fibrosis wherein said binder is a protein or protein fragment is selected from the group comprising human C5L2 protein according to SEQ ID No.: 1, a protein/peptide or fragment that is at least 60% identical to the full-length amino acid sequence of human C5L2 protein of SEQ ID No.:1, human C5aR1 protein according to SEQ ID No.: 2, a protein or fragment that is at least 60% identical to the full-length amino acid sequence of human C5aR1 protein of SEQ ID No.: 2, human C3aR protein according to SEQ ID No.: 3, a protein or fragment that is at least 60% identical to the full-length amino acid sequence of human C3aR protein as of SEQ ID No.: 3, a mouse C5L2 protein according to SEQ ID No.: 4, a protein or fragment that is at least 60% identical to the full-length amino acid sequence of mouse C5L2 protein of SEQ ID No.:4, mouse C5aR1 protein according to SEQ ID No.: 5, a protein or fragment that is at least 60% identical to the full-length amino acid sequence of mouse C5aR1 protein of SEQ ID No.: 5, mouse C3aR protein according to SEQ ID No.: 6, and a protein or fragment that is at least 60% identical to the full-length amino acid sequence of mouse C3aR protein of SEQ ID No.: 6.
16. Binder according to any of claims 1 to 15 for use in the treatment of a subject having an ocular wound and/or fibrosis wherein said binder is a protein/peptide or protein fragment and comprises at least one conserved region selected from the group comprising an amino acid sequence according to SEQ ID No.:7, an amino acid sequence according to SEQ ID No.:8, an amino acid sequence according to SEQ ID No.:9, an amino acid sequence according to SEQ ID No.:10, an amino acid sequence according to SEQ ID No.:11, an amino acid sequence according to SEQ ID No.:12, an amino acid sequence according to SEQ ID No.:13, an amino acid sequence according to SEQ ID No.:14, an amino acid sequence according to SEQ ID No.:15, an amino acid sequence according to SEQ ID No.:16, an amino acid sequence according to SEQ ID No.:17, and a protein or fragment that is at least 60% identical to any of the amino acid sequences according to SEQ ID No's.:7-17.
17. Binder according to claim 16 for use in the treatment of a subject having an ocular wound and/or fibrosis wherein said binder is a protein/peptide or protein fragment and comprises at least two conserved region selected from the group comprising an amino acid sequence according to SEQ ID No.:7, an amino acid sequence according to SEQ ID No.:8, an amino acid sequence according to SEQ ID No.:9, an amino acid sequence according to SEQ ID No.:10, an amino acid sequence according to SEQ ID No.:11, an amino acid sequence according to SEQ ID No.:12, an amino acid sequence according to SEQ ID No.:13, an amino acid sequence according to SEQ ID No.:14, an amino acid sequence according to SEQ ID No.:15, an amino acid sequence according to SEQ ID No.:16, an amino acid sequence according to SEQ ID No.:17, and a protein or fragment that is at least 60% identical to any of the amino acid sequences according to SEQ ID No's.:7-17.
18. Binder according to any of claims 15-17 for use in the treatment of a subject having an ocular wound and/or fibrosis wherein said binder is a protein/peptide or protein fragment and comprises at least one conserved region selected from the group comprising an amino acid sequence according to SEQ ID No.:18 and an amino acid sequence according to SEQ ID No.:19.
19. Composition comprising at least two binders, preferably proteins or protein fragments, according to any of claims 1 to 18 for use in the treatment of a subject having an ocular wound and/or fibrosis.
20. Composition comprising at least three proteins or protein fragments according to any of claims 1 to 19 for use in the treatment of a subject having an ocular wound and/or fibrosis.
21. Pharmaceutical composition comprising a binder according to any of claims 1-18 or a composition according to claims 19 or 20 for use in the treatment of a subject having an ocular wound and/or fibrosis.
22. Binder according to any of claims 1-18 or a composition according to claims 19 or 20 or a pharmaceutical composition of claim 21 for use in the treatment of a subject wherein said subject suffers from a disease selected from the group comprising: conjunctivitis and conjunctival scars (including ocular pemphigoid), scleritis and episcleritis, corneal scars and opacities due to corneal ulcer, keratoconjunctivitis, keratitis, bullous keratopathy, corneal degenerations, iridocyclitis and adhesions of iris and ciliary body, chorioretinal scars/fibrosis due to chorioretinal inflammation or degeneration or haemorrhage or rupture or neovascularization, fibrotic vitreoretinopathies, such as in proliferative vitreoretinopathy, retinopathy of prematurity and diabetic retinopathy; choroidal neovascularization and degenerations of the macula, secondary glaucoma, endophthalmitis, and impairments of wound healing and fibrosis after ocular surgery or trauma, including intraocular foreign bodies.
23. Binder according to any of claims 1-18 or a composition according to claims 19 or 20 or a pharmaceutical composition of claim 21 for use in the treatment of a subject wherein said subject suffers from a disease selected from the group comprising: (idiopathic) pulmonary fibrosis, dermal keloid formation, scleroderma, myelofibrosis, kidney-, pancreas- and heart-fibrosis, and fibrosis in (non)-alcoholic steatohepatosis, glomerulonephritis and (ANCA-associated) vasculitis.

### FIGURE DESCRIPTION

FIG. 1 shows the effect of inhibiting a C3a-mediated myofibroblast activation by human C5L2 protein fragment (hC5L2) using human corneal keratocytes.

FIG. 2 shows the effect of inhibiting inhibition a C5a-mediated myofibroblast activation by human C5L2 protein fragment (hC5L2) using human corneal keratocytes.

FIG. 3 shows the effect of inhibiting a C5a- and C3a-mediated myofibroblast activation by human C5L2 protein fragment (hC5L2) using human corneal keratocytes.

FIG. 4 shows the effect of inhibiting a C3a-mediated myofibroblast activation by mouse C5L2 protein fragment (mC5L2) using human corneal keratocytes.

FIG. 5 shows the effect of inhibiting a C5a-mediated myofibroblast activation by mouse C5L2 protein fragment (mC5L2) using human corneal keratocytes.

FIG. 6 shows the effect of inhibiting a C5a- and C3a-mediated myofibroblast activation by mouse C5L2 protein fragment (mC5L2) using human corneal keratocytes.

FIG. 7 shows the effect of human C5L2 protein fragment concentration on myofibroblasts in the presence of fetal bovine serum (FCS) using human corneal keratocytes

FIG. 8 shows the effect of mouse C5L2 protein fragment concentration on myofibroblasts in the presence of fetal bovine serum (FCS) using human corneal keratocytes.

FIG. 9 shows the effect of human C5L2 protein fragment concentration on myofibroblasts without fetal bovine serum using human corneal keratocytes.

FIG. 10 shows the effect of mouse C5L2 protein fragment concentration on myofibroblasts without fetal bovine serum using human corneal keratocytes.

FIG. 11 shows the effect of inhibiting a C3a-mediated myofibroblast activation by human C5L2 protein fragment (hC5L2) using human alveolar basal epithelial cells.

FIG. 12 shows the effect of inhibiting a C5a-mediated myofibroblast activation by human C5L2 protein fragment (hC5L2) using human alveolar basal epithelial cells.

FIG. 13 shows the effect of inhibiting a C5a- and C3a-mediated myofibroblast activation by human C5L2 protein fragment (hC5L2) using human alveolar basal epithelial cells.

FIG. 14 shows the effect of human C5L2 protein fragment concentration on myofibroblasts in the presence of fetal bovine serum (FCS) using human alveolar basal epithelial cells.

FIG. 15 shows the effect of inhibiting a C3a-mediated myofibroblast activation by full-length recombinant human C5a anaphylatoxin chemotactic receptor 2 (rhC5AR2/rhC5L2) using human corneal keratocytes.

FIG. 16 shows the effect of inhibiting a C5a-mediated myofibroblast activation by full-length recombinant human C5a anaphylatoxin chemotactic receptor 2 (rhC5AR2/rhC5L2) using human corneal keratocytes.

FIG. 17 shows the effect of inhibiting a C3a- and C5a-mediated myofibroblast activation by full-length recombinant human C5a anaphylatoxin chemotactic receptor 2 (rhC5AR2/rhC5L2) using human corneal keratocytes.

FIG. 18 shows the effect of full-length recombinant human C5a anaphylatoxin chemotactic receptor 2 (rhC5AR2/rhC5L2) concentration on myofibroblasts in the presence of fetal bovine serum (FCS) using human corneal keratocytes.

FIG. 19 shows the effect of full-length recombinant human C5a anaphylatoxin chemotactic receptor 2 (rhC5AR2/rhC5L2) concentration on myofibroblasts without fetal bovine serum (FCS) using human corneal keratocytes.

FIG. 20 shows the effect of inhibiting a C3a-mediated myofibroblast activation by full-length recombinant human C5a anaphylatoxin chemotactic receptor 1 (rhC5AR1) using human corneal keratocytes.

FIG. 21 shows the effect of inhibiting a C5a-mediated myofibroblast activation by full-length recombinant human C5a anaphylatoxin chemotactic receptor 1 (rhC5AR1) using human corneal keratocytes.

FIG. 22 shows the effect of inhibiting a C3a- and C5a-mediated myofibroblast activation by full-length recombinant human C5a anaphylatoxin chemotactic receptor 1 (rhC5AR1) using human corneal keratocytes.

FIG. 23 shows the effect of full-length recombinant human C5a anaphylatoxin chemotactic receptor 1 (rhC5AR1) concentration on myofibroblasts in presence of fetal bovine serum (FCS) using human corneal keratocytes.

FIG. 24 shows the effect of full-length recombinant human C5a anaphylatoxin chemotactic receptor 1 (rhC5AR1) concentration on myofibroblasts without fetal bovine serum (FCS) using human corneal keratocytes.

FIG. 25 shows the effect of inhibiting a C3a-mediated myofibroblast activation by full-length recombinant human C3a anaphylatoxin chemotactic receptor (rhC3AR) using human corneal keratocytes.

FIG. 26 shows the effect of inhibiting a C5a-mediated myofibroblast activation by full-length recombinant human C3a anaphylatoxin chemotactic receptor (rhC3AR) using human corneal keratocytes.

FIG. 27 shows the effect of inhibiting a C3a- and C5a-mediated myofibroblast activation by full-length recombinant human C3a anaphylatoxin chemotactic receptor (rhC3AR) using human corneal keratocytes.

FIG. 28 shows the effect of full-length recombinant human C3a anaphylatoxin chemotactic receptor 1 (rhC3AR) concentration on myofibroblasts in presence of fetal bovine serum (FCS) using human corneal keratocytes.

FIG. 29 shows the effect of full-length recombinant human C3a anaphylatoxin chemotactic receptor 1 (rhC3AR) concentration on myofibroblasts in without fetal bovine serum (FCS) using human corneal keratocytes.

FIG. 30 shows the effect of inhibiting a C3a-mediated myofibroblast activation by an RNA/DNA aptamer binding to human C5a using human corneal keratocytes.

FIG. 31 shows the effect of inhibiting a C5a-mediated myofibroblast activation by an RNA/DNA aptamer binding to human C5a using human corneal keratocytes.

FIG. 32 shows the effect of inhibiting a C3a- and C5a-mediated myofibroblast activation by an RNA/DNA aptamer binding to human C5a using human corneal keratocytes.

FIG. 33 shows the effect of the concentration of a RNA/DNA aptamer binding to human C5a on myofibroblasts in presence of fetal bovine serum (FCS) using human corneal keratocytes.

FIG. 34 shows the effect of the concentration of a RNA/DNA aptamer binding to human C5a on myofibroblasts without fetal bovine serum (FCS) using human corneal keratocytes.

FIG. 35 shows the effect of inhibiting a C3a-, C5a-, or C3a- and C5a-mediated myofibroblast activation by an antibody binding to human C5a (Antibody 250565) using human corneal keratocytes.

FIG. 36 shows the effect of inhibiting a C3a-, C5a-, or C3a- and C5a-mediated myofibroblast activation by antibody binding to human C5a (Antibody 308733) using human corneal keratocytes.

FIG. 37 shows the effect of inhibiting a C3a-, C5a-, or C3a- and C5a-mediated myofibroblast activation by an antibody binding to human C3a (Antibody sc28294) using human corneal keratocytes.

FIG. 38 shows the effect of inhibiting a C3a-, C5a-, or C3a- and C5a-mediated myofibroblast activation by an antibody binding to human C3a (Antibody HM1072) using human corneal keratocytes.

FIG. 39 shows the Fibrosis Grading Scores in a Corneal Alkali-Burn mouse model 20 days after Corneal Alkali-Burn, in presence or absence of mouse C5L2 protein fragment (mC5L2).

FIG. 40 shows the Items of the Cowell Fibrosis Score in a Corneal Alkali-Burn mouse model 20 days after Corneal Alkali-Burn, in presence or absence of mouse C5L2 protein fragment (mC5L2).

### EXAMPLES

### Example 1

Human C5L2 Protein Fragment Causes Inhibition of Myofibroblasts Activated by C3a

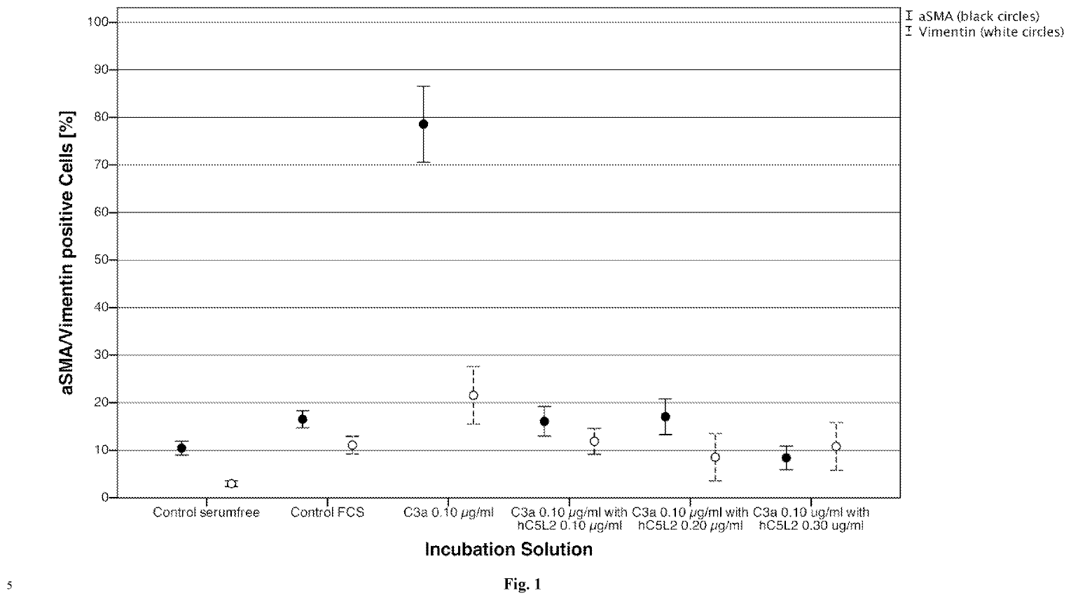

To explore the potential functional role of human C5L2 protein fragment (hC5L2), according to SEQ ID No.: 18, in the treatment of a subject having an ocular wound or fibrosis, the effect of its presence on C3a-activated myofibroblasts was examined. Human corneal keratocytes were stimulated with human C3a for 24 hours and assessed in regard to activated myofibroblasts (FIG. 1). For the detection of activated myofibroblasts aSMA antibodies were used, as well as vimentin antibodies as a marker for extracellular matrix. As a reference group, human corneal keratocytes incubated for 24 hours in DMEM growth medium with and without 10% fetal bovine serum (FCS, fetal calf serum) respectively were used. As shown in FIG. 1, C3a 0.1 μg/ml caused significant activation of myofibroblasts (measured by aSMA positive cells, 74±22%) in comparison with the reference group (serumfree: 10±11%; FCS: 16±14%; p<0.001 and p<0.001, respectively). A list of genes, attained from human corneal keratocytes and generated from a gene expression Clariom S human microarray, that have differing expression levels (fold change: ≥2 or ≤−2) after 24 hours of incubation with human C3a 0.1 μg/ml and DMEM growth medium without fetal bovine serum (serumfree control) is shown in Table 2. Incubation in the presence of human C3a and the human C5L2 protein fragment resulted in significant decrease (hC5L2 0.1 μg/ml: 16±9%; hC5L2 0.2 μg/ml: 17±11%; hC5L2 0.3 μg/ml: 8±7%), compared to C3a-activated myofibroblasts (p<0.001, p<0.001 and p<0.001, respectively). A list of genes, attained from human corneal keratocytes and generated from a gene expression Clariom S human microarray, that have differing expression levels (fold change: ≥2 or ≤−2) after 24 hours of incubation with human C3a 0.1 μg/ml and human C3a 0.1 μg/ml with human C5L2 protein fragment 0.3 μg/ml, according to SEQ ID No.: 18, is shown in Table 5. Thus, the human C5L2 protein fragment was responsible for causing inhibition of myofibroblasts activated by C3a. Bar=Standard error of the mean.

### Example 2

Human C5L2 Protein Fragment Causes Inhibition of Myofibroblasts Activated by C5a

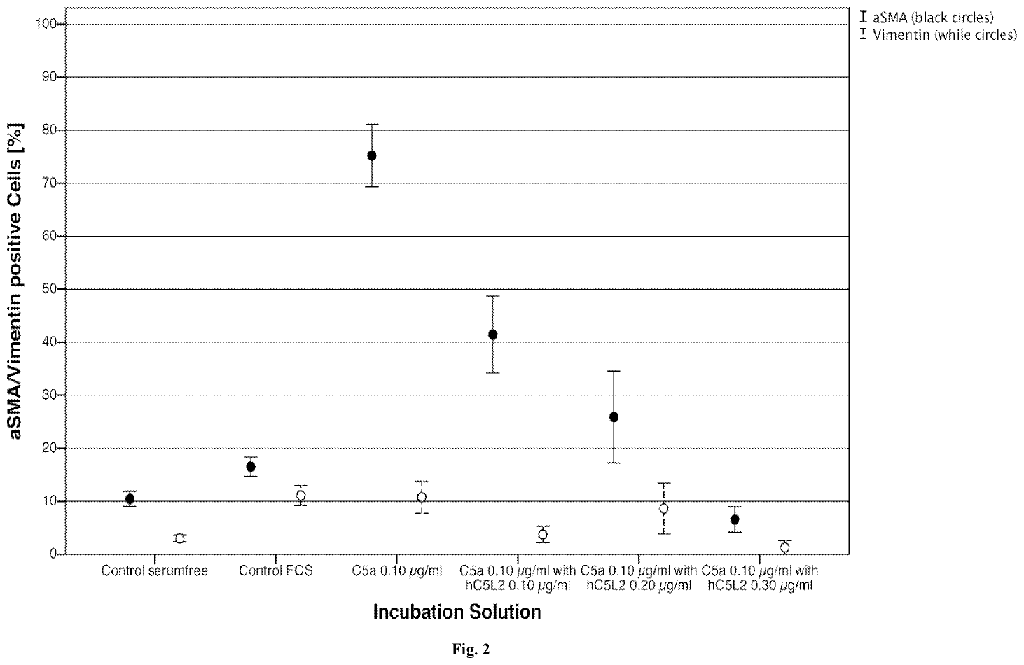

To explore the potential functional role of human C5L2 protein fragment (hC5L2), according to SEQ ID No.: 18, in the treatment of a subject having an ocular wound or fibrosis, the effect of its presence on C5a-activated myofibroblasts was examined. Human corneal keratocytes were stimulated with human C5a for 24 hours and assessed in regard to activated myofibroblasts (FIG. 2). For the detection of activated myofibroblasts aSMA antibodies were used, as well as vimentin antibodies as a marker for extracellular matrix. As a reference group, human corneal keratocytes incubated for 24 hours in DMEM growth medium with and without 10% fetal bovine serum (FCS, fetal calf serum) respectively were used. As shown in FIG. 2, C5a 0.1 μg/ml caused significant activation of myofibroblasts (measured by aSMA positive cells, 77±23%) in comparison with the reference group (serumfree: 10±11%; FCS: 16±14%; p<0.001 and p<0.001, respectively). A list of genes, attained from human corneal keratocytes and generated from a gene expression Clariom S human microarray, that have differing expression levels (fold change: ≥2 or ≤−2) after 24 hours of incubation with human C5a 0.1 μg/ml and DMEM growth medium without fetal bovine serum (serumfree control) is shown in Table 3. Incubation in the presence of C5a and the human C5L2 protein fragment resulted in significant decrease of activated myofibroblasts (hC5L2 0.1 μg/ml: 41±22%; hC5L2 0.2 μg/ml: 26±26%; hC5L2 0.3 μg/ml: 7±7%), compared to C5a-activated myofibroblasts (p=0.001, p<0.001 and p<0.001, respectively). A list of genes, attained from human corneal keratocytes and generated from a gene expression Clariom S human microarray, that have differing expression levels (fold change: ≥2 or ≤−2) after 24 hours of incubation with human C5a 0.1 μg/ml and human C5a 0.1 μg/ml with human C5L2 protein fragment 0.3 μg/ml, according to SEQ ID No.: 18, is shown in Table 6. Thus, the human C5L2 protein fragment was responsible for causing inhibition of myofibroblasts activated by C5a. Bar=Standard error of the mean.

### Example 3

Human C5L2 Protein Fragment Causes Inhibition of Myofibroblasts Activated by C5a and C3a

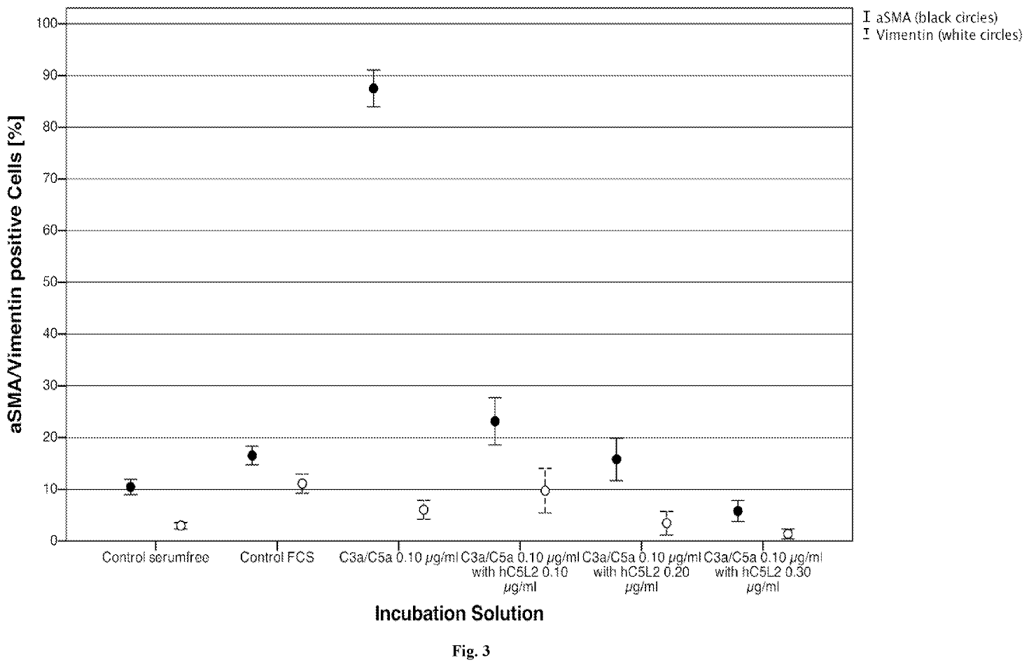

To explore the potential functional role of human C5L2 protein fragment, according to SEQ ID No.: 18, in the treatment of a subject having an ocular wound or fibrosis, the effect of its presence on C5a/C3a-activated myofibroblasts was examined. Human corneal keratocytes were stimulated with human C5a and human C3a respectively for 24 hours and assessed in regard to activated myofibroblasts (FIG. 3). For the detection of activated myofibroblasts aSMA antibodies were used, as well as vimentin antibodies as marker for extracellular matrix. As a reference group, human corneal keratocytes incubated for 24 hours in DMEM growth medium with and without 10% fetal bovine serum (FCS, fetal calf serum) respectively were used. As shown in FIG. 3, C5a and C3a, both at a concentration of 0.1 μg/ml, caused significant activation of myofibroblasts (measured by aSMA positive cells, 87±11%) in comparison with the reference group (serumfree: 10±11%; FCS: 16±14%; p<0.001 and p<0.001, respectively). A list of genes, attained from human corneal keratocytes and generated from a gene expression Clariom S human microarray, that have differing expression levels (fold change: ≥2 or ≤−2) after 24 hours of incubation with human C3a and C5a, both at a concentration of 0.1 μg/ml, and DMEM growth medium without fetal bovine serum (serumfree control) is shown in Table 4. Incubation in the presence of C3a, C5a and the human C5L2 protein fragment resulted in significant decrease of activated myofibroblasts (hC5L2 0.1 μg/ml: 23±14%; hC5L2 0.2 μg/ml: 16±12%; hC5L2 0.3 μg/ml: 6±6%), compared to C5a- and C3a-activated myofibroblasts (p<0.001, p<0.001 and p<0.001, respectively). A list of genes, attained from human corneal keratocytes and generated from a gene expression Clariom S human microarray, that have differing expression levels (fold change: ≥2 or ≤−2) after 24 hours of incubation with human C3a and C5a, both 0.1 μg/ml, and human C3a and C5a, both 0.1 μg/ml, with human C5L2 protein fragment 0.3 μg/ml, according to SEQ ID No.: 18, is shown in Table 7. Thus, the human C5L2 protein fragment was responsible for causing inhibition of myofibroblasts activated by C5a and C3a. Bar=Standard error of the mean.

### Example 4

Mouse C5L2 Protein Fragment Causes Inhibition of Myofibroblasts Activated by C3a

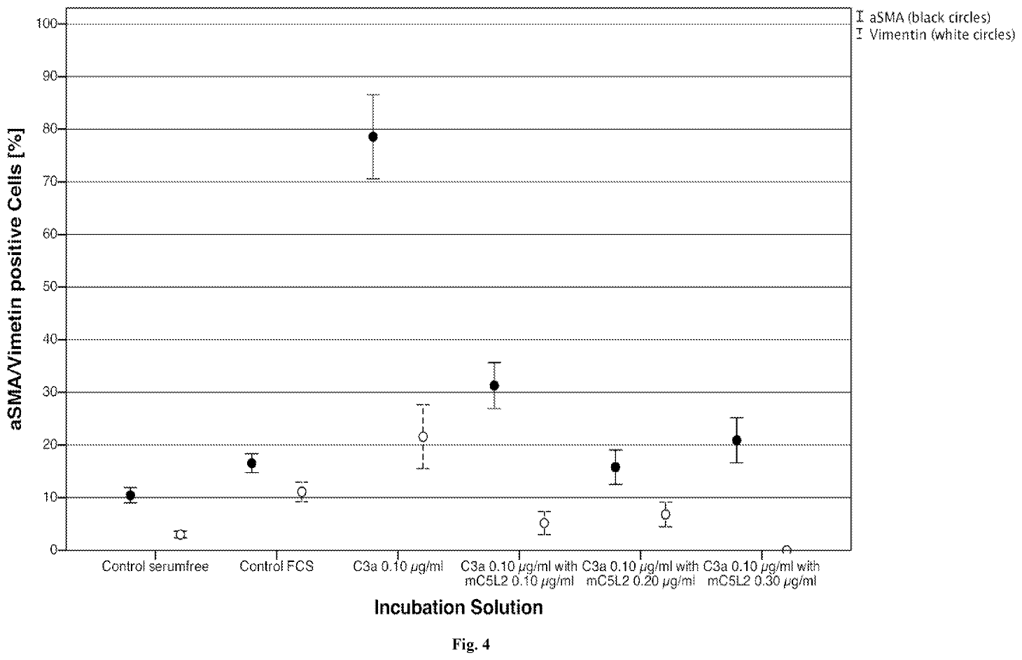

To explore the potential functional role of mouse C5L2 protein fragment (mC5L2), according to SEQ ID No.: 19, in the treatment of a subject having an ocular wound or fibrosis, the effect of its presence on C3a-activated myofibroblasts was examined. Human corneal keratocytes were stimulated with human C3a over 24 hours and assessed in regard to activated myofibroblasts (FIG. 4). For the detection of activated myofibroblasts aSMA antibodies were used, as well as vimentin antibodies as marker for extracellular matrix. As a reference group, human corneal keratocytes incubated for 24 hours in DMEM growth medium with and without 10% fetal bovine serum (FCS, fetal calf serum) respectively were used. As shown in FIG. 4, C3a 0.1 μg/ml caused significant activation of myofibroblasts (measured by aSMA positive cells, 74±22%) in comparison with the reference group (serumfree: 10±11%; FCS: 16±14%; p<0.001 and p<0.001, respectively). Incubation in the presence of C3a and the mouse C5L2 protein fragment resulted in significant decrease of activated myofibroblasts (mC5L2 0.1 μg/ml: 31±13%; mC5L2 0.2 μg/ml: 16±10%; mC5L2 0.3 μg/ml: 21±13%), compared to C3a-activated myofibroblasts (p<0.001, p<0.001 and p<0.001, respectively). Thus, the mouse C5L2 protein fragment was responsible for causing inhibition of myofibroblasts activated by C3a. Bar=Standard error of the mean.

### Example 5

Mouse C5L2 Protein Fragment Causes Inhibition of Myofibroblasts Activated by C5a

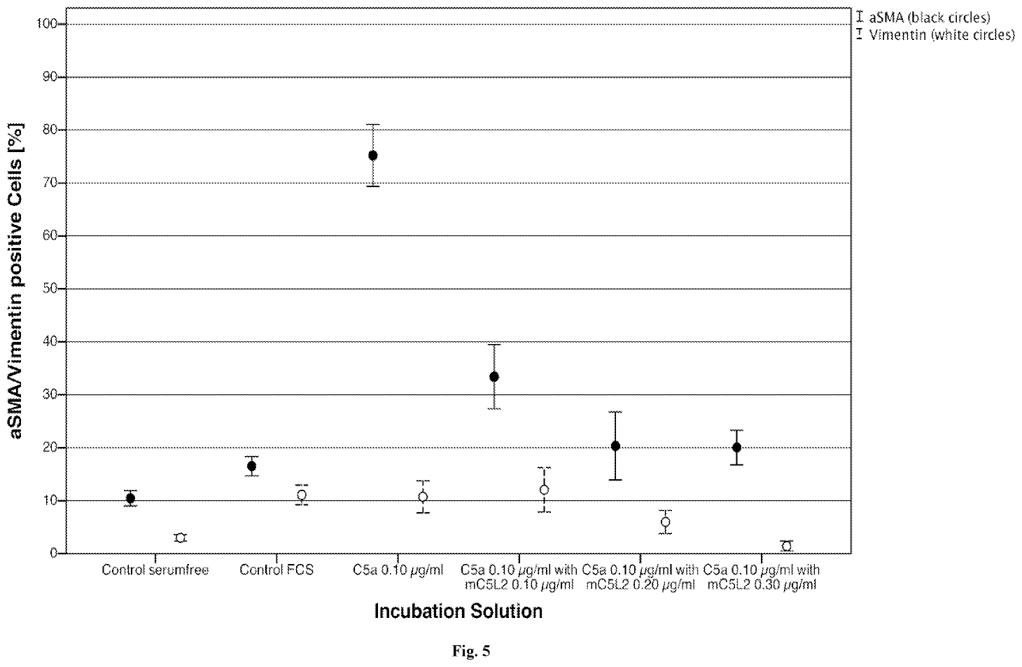

To explore the potential functional role of mouse C5L2 protein fragment (mC5L2), according to SEQ ID No.: 19, in the treatment of a subject having an ocular wound or fibrosis, the effect of its presence on C5a-activated myofibroblasts was examined. Human corneal keratocytes were stimulated with human C5a over 24 hours and assessed in regard to activated myofibroblasts (FIG. 5). For the detection of activated myofibroblasts aSMA antibodies were used, as well as vimentin antibodies as marker for extracellular matrix. As a reference group, human corneal keratocytes incubated for 24 hours in DMEM growth medium with and without 10% fetal bovine serum (FCS, fetal calf serum) respectively were used. As shown in FIG. 5, C5a 0.1 μg/ml caused significant activation of myofibroblasts (measured by aSMA positive cells, 77±23%) in comparison with the reference group (serumfree: 10±11%; FCS: 16±14%; p<0.001 and p<0.001, respectively). Incubation in the presence of C5a and the mouse C5L2 protein fragment resulted in significant decrease of activated myofibroblasts (mC5L2 0.1 μg/ml: 33±18%; mC5L2 0.2 μg/ml: 20±19%; mC5L2 0.3 μg/ml: 20±10%), compared to C5a-activated myofibroblasts (p<0.001, p<0.001 and p<0.001, respectively). Thus, the mouse C5L2 protein fragment was responsible for causing inhibition of myofibroblasts activated by C5a. Bar=Standard error of the mean.

### Example 6

Mouse C5L2 Protein Fragment Causes Inhibition of Myofibroblasts Activated by C5a and C3a

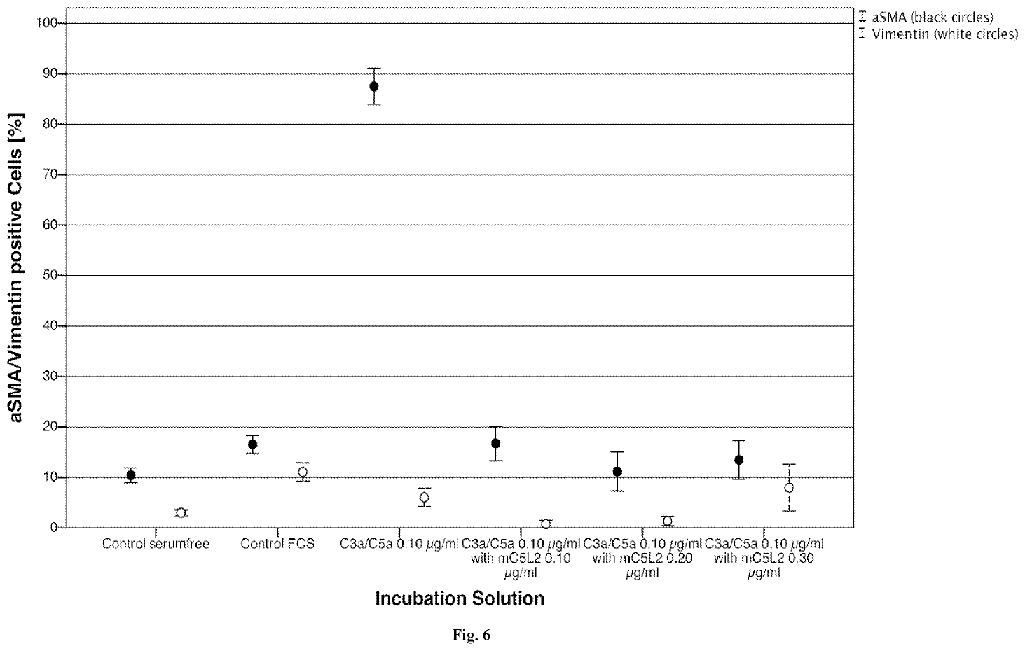

To explore the potential functional role of mouse C5L2 protein fragment (mC5L2), according to SEQ ID No.: 19, in the treatment of a subject having an ocular wound or fibrosis, the effect of its presence on C5a/C3a-activated myofibroblasts was examined. Human corneal keratocytes were stimulated with human C5a and human C3a respectively for 24 hours and assessed in regard to activated myofibroblasts (FIG. 6). For the detection of activated myofibroblasts aSMA antibodies were used, as well as vimentin antibodies as marker for extracellular matrix. As a reference group, human corneal keratocytes incubated for 24 hours in DMEM growth medium with and without 10% fetal bovine serum (FCS, fetal calf serum) respectively were used. As shown in FIG. 6, C5a and C3a, both at a concentration of 0.1 μg/ml, caused significant activation of myofibroblasts (measured by aSMA positive cells, 87±11% respectively) in comparison with the reference group (serumfree: 10±11%; FCS: 16±14%; p<0.001 and p<0.001, respectively). Incubation in the presence of C3a, C5a and the mouse C5L2 protein fragment resulted in significant decrease of activated myofibroblasts (mC5L2 0.1 μg/ml: 17±10%; mC5L2 0.2 μg/ml: 11±12%; mC5L2 0.3 μg/ml: 13±11%), compared to C5a- and C3a-activated myofibroblasts (p<0.001, p<0.001 and p<0.001, respectively). Thus, the mouse C5L2 protein fragment was responsible for causing inhibition of myofibroblasts activated by C5a and C3a. Bar=Standard error of the mean.

### Example 7

The Effect of Human C5L2 Protein Fragment Concentration on Myofibroblasts in the Presence of Fetal Bovine Serum

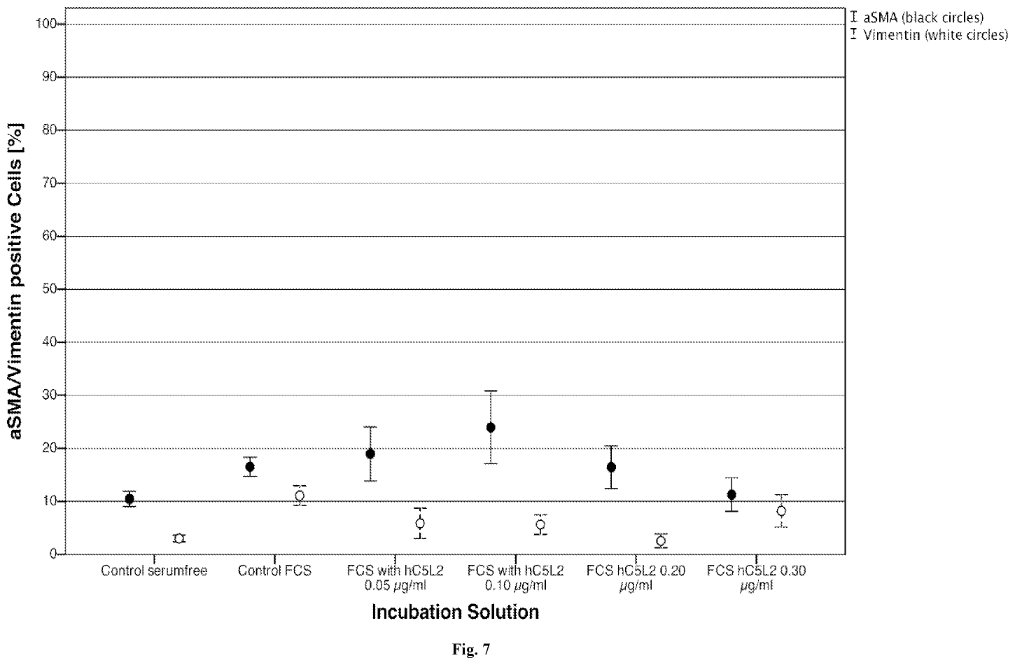

To explore the potential functional role of human C5L2 protein fragment (hC5L2), according to SEQ ID No.: 18, in the treatment of a subject having an ocular wound or fibrosis, the effect of its concentration on myofibroblasts was examined. Human corneal keratocytes were incubated for 24 hours in DMEM growth medium with 10% fetal bovine serum (FCS, fetal calf serum) and human C5L2 protein fragment in different concentrations (FIG. 7). For the detection of activated myofibroblasts aSMA antibodies were used, as well as vimentin antibodies as marker for extracellular matrix. As a reference group, human corneal keratocytes incubated for 24 hours in DMEM growth medium with and without fetal bovine serum respectively were used (serumfree: 10±11%; FCS: 16±14%). As shown in FIG. 7, human C5L2 protein fragment was found to have a slight positive effect on myofibroblasts activation in small concentrations (hC5L2 0.05 μg/ml: 19±15%; hC5L2 0.1 μg/ml: 24±21%; hC5L2 0.2 μg/ml: 16±12%), whereas inhibition of myofibroblasts was observed in higher concentrations (hC5L2 0.3 μg/ml: 11±10%). Yet, compared to 10% FCS incubated human corneal keratocytes, differences remained insignificant (p=0.554, p=0.136, p=0.918 and p=0.345, respectively).

### Example 8

The Effect of Mouse C5L2 Protein Fragment Concentration on Myofibroblasts in the Presence of Fetal Bovine Serum

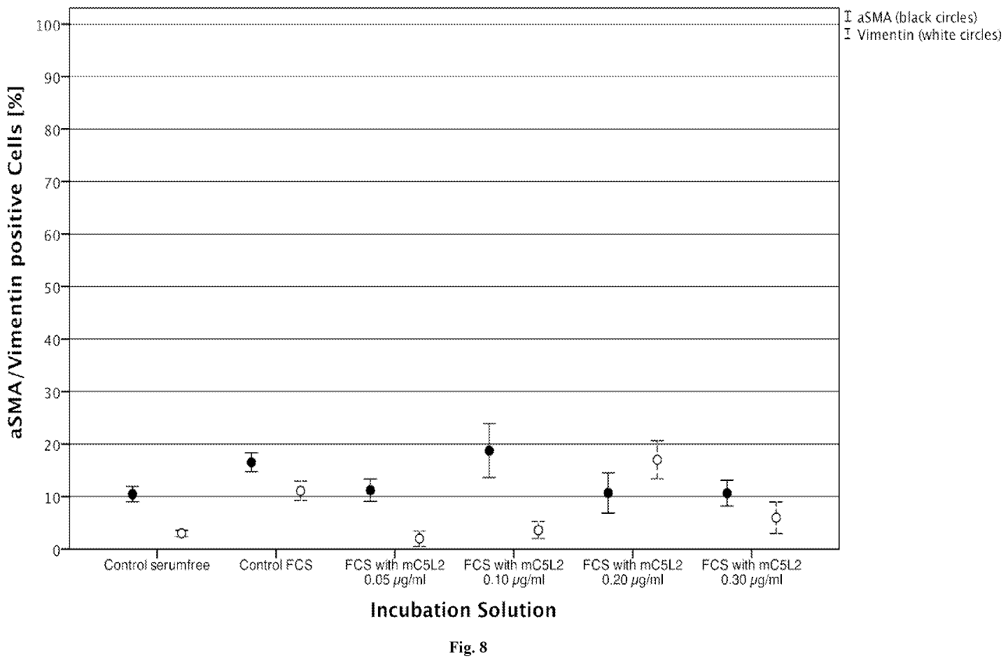

To explore the potential functional role of mouse C5L2 protein fragment (mC5L2), according to SEQ ID No.: 19, in the treatment of a subject having an ocular wound or fibrosis, the effect of its concentration on myofibroblasts was examined. Human corneal keratocytes were incubated for 24 hours in DMEM growth medium with 10% fetal bovine serum (FCS, fetal calf serum) and mouse C5L2 protein fragment in different concentrations (FIG. 8). For the detection of activated myofibroblasts aSMA antibodies were used, as well as vimentin antibodies as marker for extracellular matrix. As a reference group, human corneal keratocytes incubated for 24 hours in DMEM growth medium with and without fetal bovine serum respectively were used (serumfree: 10±11%; FCS: 16±14%). As shown in FIG. 8, mouse C5L2 protein fragment was found to have a slightly positive effect on myofibroblasts activation in small concentrations (mC5L2 0.05 μg/ml: 11±6%; mC5L2 0.1 μg/ml: 19±15%; mC5L2 0.2 μg/ml: 11±12%), whereas inhibition of myofibroblasts was observed in higher concentrations (mC5L2 0.3 μg/ml: 11±7%). Yet, compared to 10% FCS incubated human corneal keratocytes, differences remained insignificant (p=0.101, p=0.580, p=0.293 and p=0.277, respectively).

### Example 9

The Effect of Human C5L2 Protein Fragment Concentration on Myofibroblasts without Fetal Bovine Serum

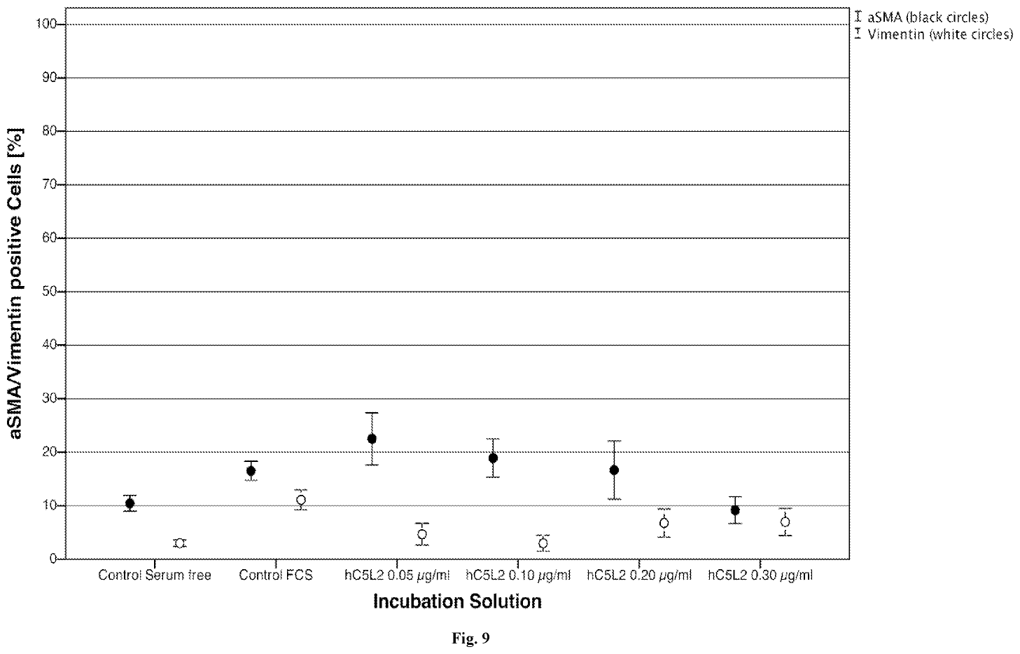

To explore the potential functional role of human C5L2 protein fragment (hC5L2), according to SEQ ID No.: 18, in the treatment of a subject having an ocular wound or fibrosis, the effect of its concentration on myofibroblasts was examined. Human corneal keratocytes were incubated for 24 hours in DMEM growth medium without fetal bovine serum and with human C5L2 protein fragment in different concentrations (FIG. 9). For the detection of activated myofibroblasts aSMA antibodies were used, as well as vimentin antibodies as marker for extracellular matrix. As a reference group, human corneal keratocytes incubated for 24 hours in DMEM growth medium with and without 10% fetal bovine serum (FCS, fetal calf serum) respectively were used (serumfree: 10±11%; FCS: 16±14%). As shown in FIG. 9, human C5L2 protein fragment was found to have a slightly positive effect on myofibroblasts activation, compared to DMEM without FCS incubated human corneal keratocytes (serumfree control), in small concentrations (hC5L2 0.05 μg/ml: 23±15%; hC5L2 0.1 μg/ml: 19±11%; p=0.005 and p=0.039, respectively), whereas inhibition of myofibroblasts was observed in higher concentrations and did not reveal a difference to the serumfree control (hC5L2 0.2 μg/ml: 17±16%; hC5L2 0.3 μg/ml: 9±8%; p=0.150 and p=0.755, respectively). A list of genes, attained from human corneal keratocytes and generated from a gene expression Clariom S human microarray, that have differing expression levels (fold change: ≥2 or ≤−2) after 24 hours of incubation with human C5L2 protein fragment 0.3 μg/ml, according to SEQ ID No.: 18, and DMEM growth medium without fetal bovine serum (serumfree control) is shown in Table 8.

### Example 10

The Effect of Mouse C5L2 Protein Fragment Concentration on Myofibroblasts without Fetal Bovine Serum

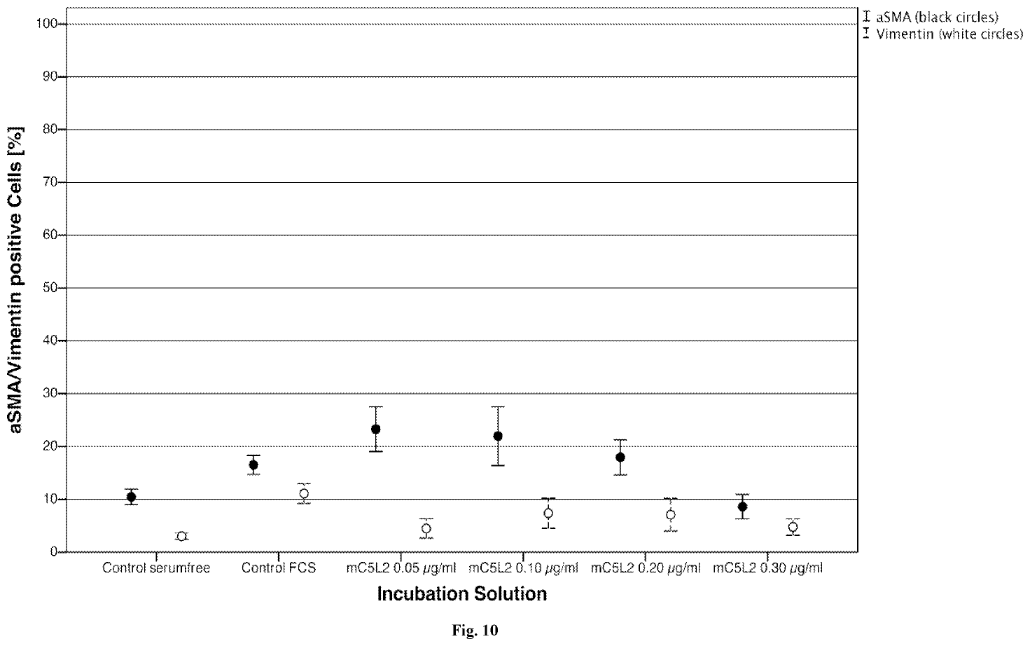

To explore the potential functional role of mouse C5L2 protein fragment (mC5L2), according to SEQ ID No.: 19, in the treatment of a subject having an ocular wound or fibrosis, the effect of its concentration on myofibroblasts was examined. Human corneal keratocytes were incubated for 24 hours in DMEM growth medium without fetal bovine serum and with mouse C5L2 protein fragment in different concentrations (FIG. 10). For the detection of activated myofibroblasts aSMA antibodies were used, as well as vimentin antibodies as marker for extracellular matrix. As a reference group, human corneal keratocytes incubated for 24 hours in DMEM growth medium with and without 10% fetal bovine serum (FCS, fetal calf serum) respectively were used (serumfree: 10±11%; FCS: 16±14%). As shown in FIG. 10, mouse C5L2 protein fragment was found to have a slight positive effect on myofibroblasts activation, compared to DMEM without FCS incubated human corneal keratocytes (serumfree control), in small concentrations (mC5L2 0.05 μg/ml: 23±13%; mC5L2 0.1 μg/ml: 22±17%; p=0.003 and p=0.009, respectively), whereas inhibition of myofibroblasts was observed in higher concentrations and did not reveal a difference to the serumfree control (hC5L2 0.2 μg/ml: 18±10%; hC5L2 0.3 μg/ml: 9±7%; p=0.064 and p=0.647, respectively).

### Example 11

Human C5L2 Protein Fragment Causes Inhibition of Myofibroblasts Activated by C3a

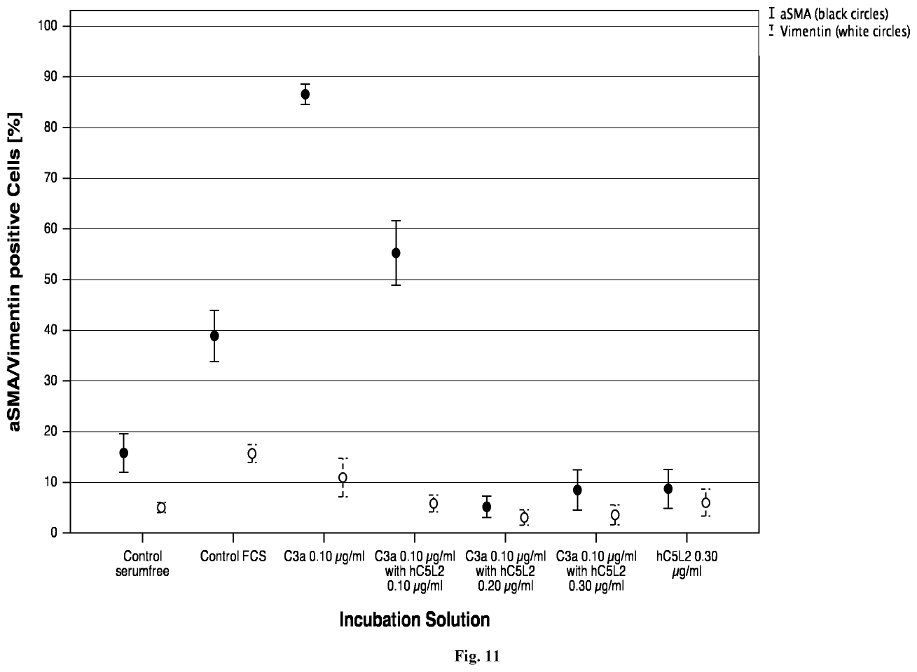

To explore the potential functional role of human C5L2 protein fragment (hC5L2), according to SEQ ID No.: 18, in the treatment of a subject having a pulmonary fibrosis, the effect of its presence on C3a-activated myofibroblasts was examined. Human alveolar basal epithelial cells (A549 cells) were stimulated with human C3a for 24 hours and assessed in regard to activated myofibroblasts (FIG. 11). For the detection of activated myofibroblasts aSMA antibodies were used, as well as vimentin antibodies as a marker for extracellular matrix. As a reference group, human alveolar basal epithelial cells (A549 cells) incubated for 24 hours in DMEM growth medium with and without 10% fetal bovine serum (FCS, fetal calf serum) respectively were used. As shown in FIG. 11, C3a 0.1 μg/ml caused significant activation of myofibroblasts (measured by aSMA positive cells, 87±6%) in comparison with the reference group (serumfree: 16±16%; FCS: 39±21%; p<0.001 and p<0.001, respectively). Incubation in the presence of human C3a and the human C5L2 protein fragment resulted in significant decrease (hC5L2 0.1 μg/ml: 55±19%; hC5L2 0.2 μg/ml: 5±6%; hC5L2 0.3 μg/ml: 8±12%), compared to C3a-activated myofibroblasts (p=0.001, p<0.001 and p<0.001, respectively). As shown in FIG. 11, the intrinsic effect of the human C5L2 protein fragment on the myofibroblast activation did not reveal a difference to the serumfree control (hC5L2 0.3 μg/ml: 9±11%; p=0.250). Thus, the human C5L2 protein fragment was responsible for causing inhibition of myofibroblasts activated by C3a. Bar=Standard error of the mean.

### Example 12

Human C5L2 Protein Fragment Causes Inhibition of Myofibroblasts Activated by C5a

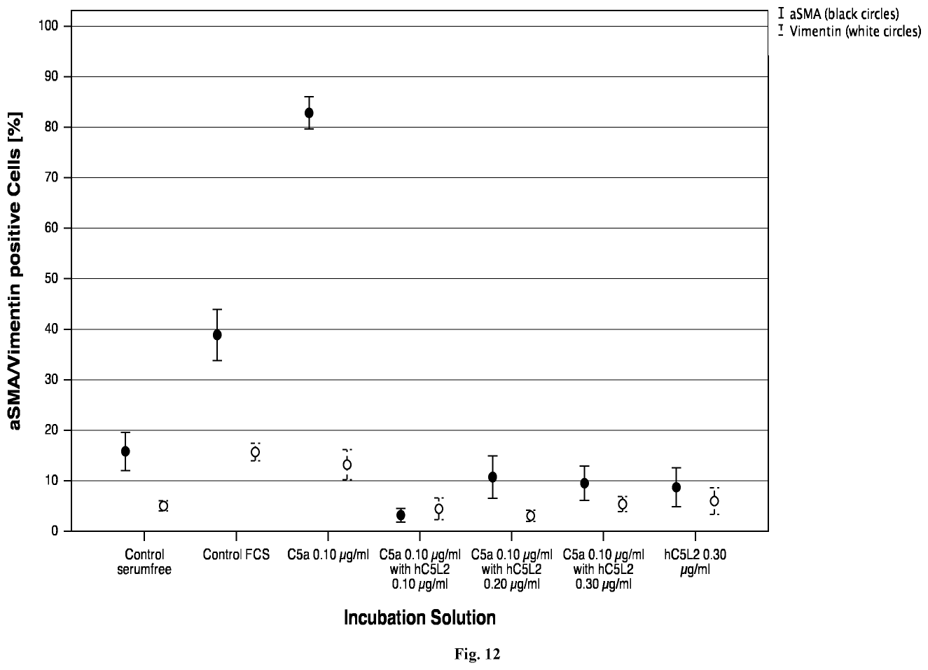

To explore the potential functional role of human C5L2 protein fragment (hC5L2), according to SEQ ID No.: 18, in the treatment of a subject having a pulmonary fibrosis, the effect of its presence on C5a-activated myofibroblasts was examined. Human alveolar basal epithelial cells (A549 cells) were stimulated with human C5a for 24 hours and assessed in regard to activated myofibroblasts (FIG. 12). For the detection of activated myofibroblasts aSMA antibodies were used, as well as vimentin antibodies as a marker for extracellular matrix. As a reference group, human alveolar basal epithelial cells (A549 cells) incubated for 24 hours in DMEM growth medium with and without 10% fetal bovine serum (FCS, fetal calf serum) respectively were used. As shown in FIG. 12, C5a 0.1 μg/ml caused significant activation of myofibroblasts (measured by aSMA positive cells, 83±10%) in comparison with the reference group (serumfree: 16±16%; FCS: 39±21%; p<0.001 and p<0.001, respectively). Incubation in the presence of human C5a and the human C5L2 protein fragment resulted in significant decrease (hC5L2 0.1 μg/ml: 3±4%; hC5L2 0.2 μg/ml: 11±13%; hC5L2 0.3 μg/ml: 10±10%), compared to C5a-activated myofibroblasts (p<0.001, p<0.001 and p<0.001, respectively). As shown in FIG. 12, the intrinsic effect of the human C5L2 protein fragment on the myofibroblast activation did not reveal a difference to the serumfree control (hC5L2 0.3 μg/ml: 9±11%; p=0.250). Thus, the human C5L2 protein fragment was responsible for causing inhibition of myofibroblasts activated by C5a. Bar=Standard error of the mean.

### Example 13

Human C5L2 Protein Fragment Causes Inhibition of Myofibroblasts Activated by C5a and C3a

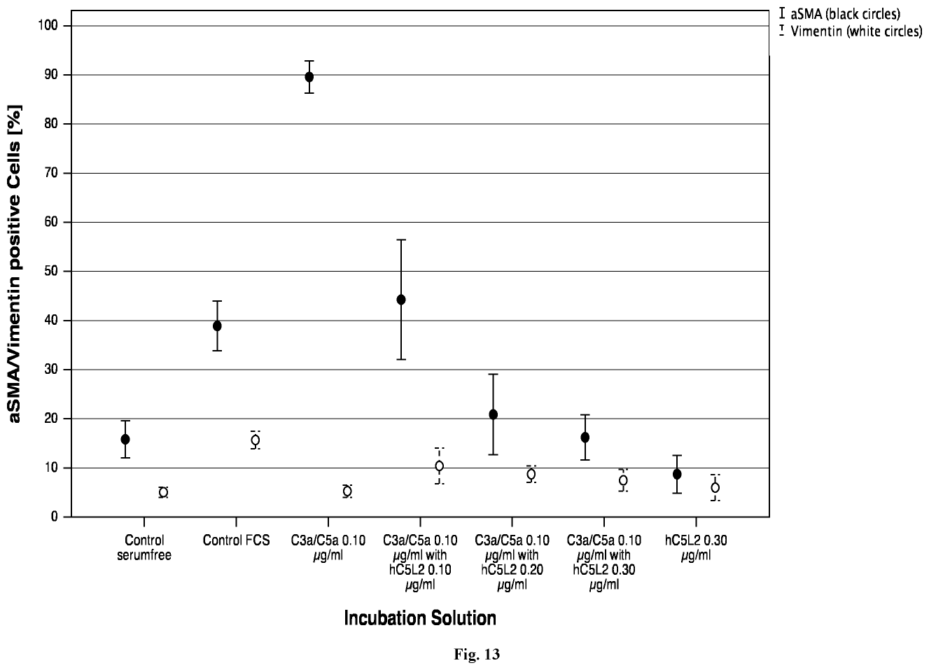

To explore the potential functional role of human C5L2 protein fragment (hC5L2), according to SEQ ID No.: 18, in the treatment of a subject having a pulmonary fibrosis, the effect of its presence on C5a/C3a-activated myofibroblasts was examined. Human alveolar basal epithelial cells (A549 cells) were stimulated with human C5a and human C3a for 24 hours and assessed in regard to activated myofibroblasts (FIG. 13). For the detection of activated myofibroblasts aSMA antibodies were used, as well as vimentin antibodies as a marker for extracellular matrix. As a reference group, human alveolar basal epithelial cells (A549 cells) incubated for 24 hours in DMEM growth medium with and without 10% fetal bovine serum (FCS, fetal calf serum) respectively were used. As shown in FIG. 13, C5a and C3a, both at a concentration of 0.1 μg/ml, caused significant activation of myofibroblasts (measured by aSMA positive cells, 90±10%) in comparison with the reference group (serumfree: 16±16%; FCS: 39±21%; p<0.001 and p<0.001, respectively). Incubation in the presence of human C3a, C5a and the human C5L2 protein fragment resulted in significant decrease (hC5L2 0.1 μg/ml: 44±37%; hC5L2 0.2 μg/ml: 21±25%; hC5L2 0.3 μg/ml: 16±14%), compared to C5a and C3a-activated myofibroblasts (p=0.006, p<0.001 and p<0.001, respectively). As shown in FIG. 13, the intrinsic effect of the human C5L2 protein fragment on the myofibroblast activation did not reveal a difference to the serumfree control (hC5L2 0.3 μg/ml: 9±11%; p=0.250). Thus, the human C5L2 protein fragment was responsible for causing inhibition of myofibroblasts activated by C5a and C3a. Bar=Standard error of the mean.

### Example 14

Human C5L2 Protein Fragment Causes Inhibition of Myofibroblasts in the Presence of Fetal Bovine Serum

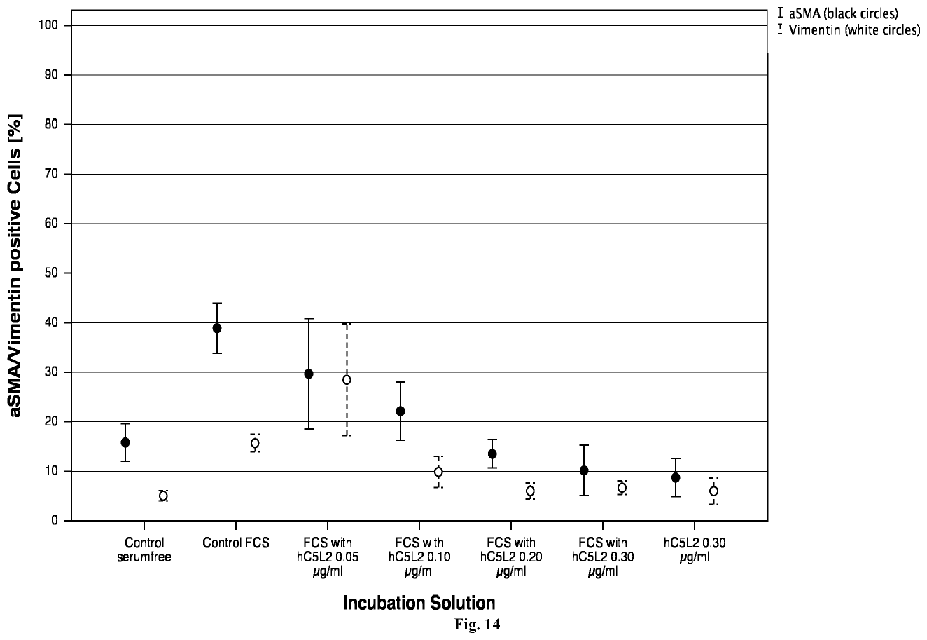

To explore the potential functional role of human C5L2 protein fragment (hC5L2), according to SEQ ID No.: 18, in the treatment of a subject having a pulmonary fibrosis, the effect of its concentration on myofibroblasts was examined. Human alveolar basal epithelial cells (A549 cells) were incubated for 24 hours in DMEM growth medium with 10% fetal bovine serum (FCS, fetal calf serum) and human C5L2 protein fragment in different concentrations (FIG. 14). For the detection of activated myofibroblasts aSMA antibodies were used, as well as vimentin antibodies as marker for extracellular matrix. As a reference group, human alveolar basal epithelial cells (A549 cells) incubated for 24 hours in DMEM growth medium with and without fetal bovine serum respectively were used (serumfree: 16±16%; FCS: 39±21%). As shown in FIG. 14, human C5L2 protein fragment was found to have a slight positive effect on myofibroblasts activation in small concentrations (hC5L2 0.05 μg/ml: 30±34% and hC5L2 0.1 μg/ml: 22±18%), whereas inhibition of myofibroblasts was observed in higher concentrations (hC5L2 0.2 μg/ml: 14±9% and hC5L2 0.3 μg/ml: 10±15%). Yet, compared to 10% FCS incubated human corneal keratocytes, differences remained insignificant (p=0.268, p=0.360, p=0.693 and p=0.390, respectively). As shown in FIG. 14, the intrinsic effect of the human C5L2 protein fragment on the myofibroblast activation did not reveal a difference to the serumfree control (hC5L2 0.3 μg/ml: 9±11%; p=0.250).

### Example 15

Full-Length Recombinant Human C5a Anaphylatoxin Chemotactic Receptor 2 (rhC5AR2/rhC5L2) Protein Causes Inhibition of Myofibroblasts Activated by C3a

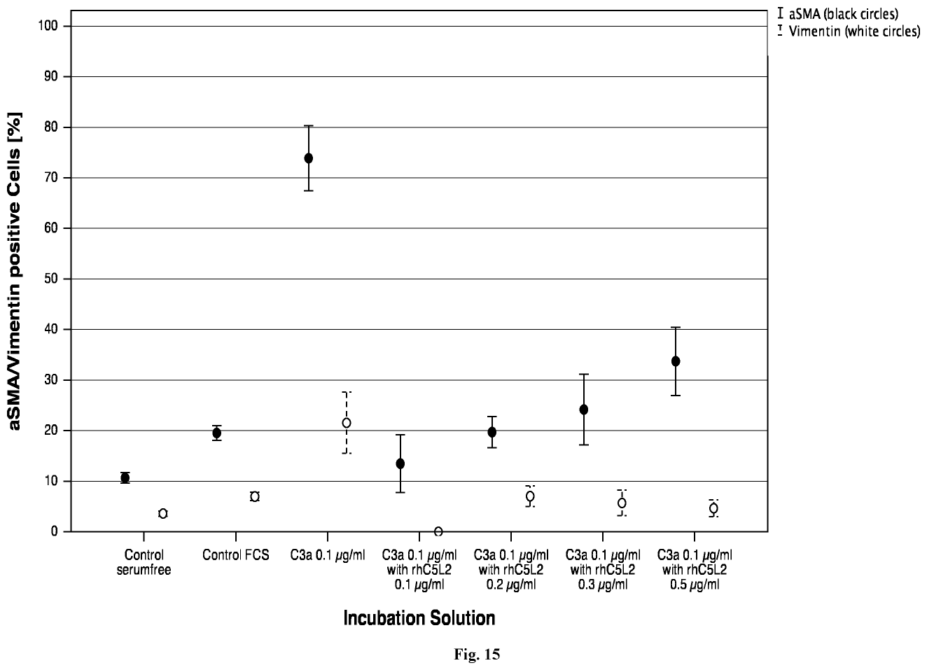

To explore the potential functional role of the rhC5L2 protein, according to SEQ ID No.: 1, in the treatment of a subject having an ocular wound or fibrosis, the effect of its presence on C3a-activated myofibroblasts was examined. Human corneal keratocytes were stimulated with human C3a for 24 hours and assessed in regard to activated myofibroblasts (FIG. 15). For the detection of activated myofibroblasts aSMA antibodies were used, as well as vimentin antibodies as a marker for extracellular matrix. As a reference group, human corneal keratocytes incubated for 24 hours in DMEM growth medium with and without 10% fetal bovine serum (FCS, fetal calf serum) respectively were used. As shown in FIG. 15, C3a 0.1 μg/ml caused significant activation of myofibroblasts (measured by aSMA positive cells, 74±22%) in comparison with the reference group (serumfree: 11±14%; FCS: 20±19%; p<0.001 and p<0.001, respectively). Incubation in the presence of human C3a and the human rhC5L2 protein resulted in significant decrease (rhC5L2 0.1 μg/ml: 13±17%; rhC5L2 0.2 μg/ml: 20±9%; rhC5L2 0.3 μg/ml: 24±21%; rhC5L2 0.5 μg/ml: 34±20%), compared to C3a-activated myofibroblasts (p<0.001, p<0.001, p<0.001 and p<0.005, respectively). Thus, the rhC5L2 protein was responsible for causing inhibition of myofibroblasts activated by C3a. Bar=Standard error of the mean.

### Example 16

Full-Length Recombinant Human C5a Anaphylatoxin Chemotactic Receptor 2 (rhC5AR2/rhC5L2) Protein Causes Inhibition of Myofibroblasts Activated by C5a

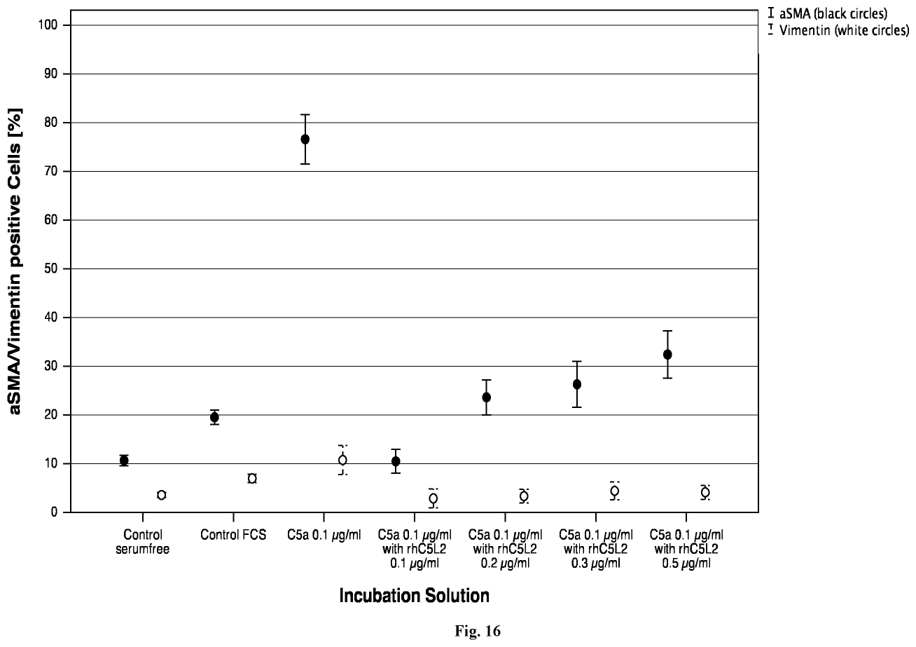

To explore the potential functional role of the rhC5L2 protein, according to SEQ ID No.: 1, in the treatment of a subject having an ocular wound or fibrosis, the effect of its presence on C5a-activated myofibroblasts was examined. Human corneal keratocytes were stimulated with human C5a for 24 hours and assessed in regard to activated myofibroblasts (FIG. 16). For the detection of activated myofibroblasts aSMA antibodies were used, as well as vimentin antibodies as a marker for extracellular matrix. As a reference group, human corneal keratocytes incubated for 24 hours in DMEM growth medium with and without 10% fetal bovine serum (FCS, fetal calf serum) respectively were used. As shown in FIG. 16, C5a 0.1 μg/ml caused significant activation of myofibroblasts (measured by aSMA positive cells, 77±23%) in comparison with the reference group (serumfree: 11±14%; FCS: 20±19%; p<0.001 and p<0.001, respectively). Incubation in the presence of human C5a and the human rhC5L2 protein resulted in significant decrease (rhC5L2 0.1 μg/ml: 11±7%; rhC5L2 0.2 μg/ml: 24±11%; rhC5L2 0.3 μg/ml: 26±14%; rhC5L2 0.5 μg/ml: 32±15%), compared to C5a-activated myofibroblasts (p<0.001, p<0.001, p<0.001 and p<0.001, respectively). Thus, the rhC5L2 protein was responsible for causing inhibition of myofibroblasts activated by C5a. Bar=Standard error of the mean.

### Example 17

Full-Length Recombinant Human C5a Anaphylatoxin Chemotactic Receptor 2 (rhC5AR2/rhC5L2) Protein Causes Inhibition of Myofibroblasts Activated by C5a and C3a

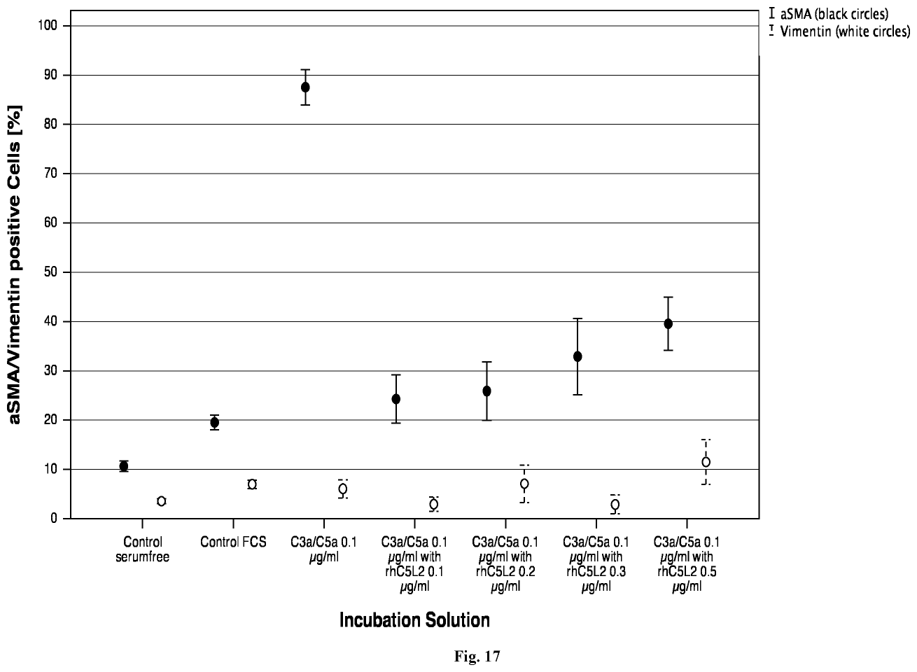

To explore the potential functional role of the rhC5L2 protein, according to SEQ ID No.: 1, in the treatment of a subject having an ocular wound or fibrosis, the effect of its presence on C5a/C3a-activated myofibroblasts was examined. Human corneal keratocytes were stimulated with human C5a and human C3a for 24 hours and assessed in regard to activated myofibroblasts (FIG. 17). For the detection of activated myofibroblasts aSMA antibodies were used, as well as vimentin antibodies as a marker for extracellular matrix. As a reference group, human corneal keratocytes incubated for 24 hours in DMEM growth medium with and without 10% fetal bovine serum (FCS, fetal calf serum) respectively were used. As shown in FIG. 17, C5a and C3a, both at a concentration of 0.1 μg/ml, caused significant activation of myofibroblasts (measured by aSMA positive cells, 88±11%) in comparison with the reference group (serumfree: 11±14%; FCS: 20±19%; p<0.001 and p<0.001, respectively). Incubation in the presence of human C3a, C5a and the human rhC5L2 protein resulted in significant decrease (rhC5L2 0.1 μg/ml: 24±15%; rhC5L2 0.2 μg/ml: 26±18%; rhC5L2 0.3 μg/ml: 33±23%; rhC5L2 0.5 μg/ml: 40±16%), compared to C5a and C3a-activated myofibroblasts (p<0.001, p<0.001, p<0.001 and p<0.001, respectively). Thus, the rhC5L2 protein was responsible for causing inhibition of myofibroblasts activated by C5a and C3a. Bar=Standard error of the mean.

### Example 18

The Effect of the Full-Length Recombinant Human C5a Anaphylatoxin Chemotactic Receptor 2 (rhC5AR2/rhC5L2) Protein Concentration on Myofibroblasts in the Presence of Fetal Bovine Serum

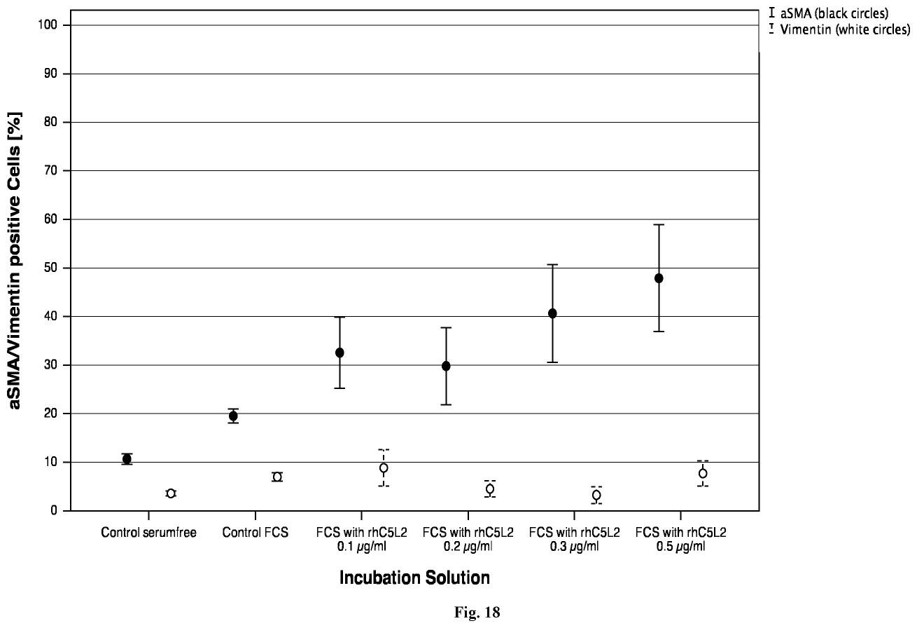

To explore the potential functional role of the human rhC5L2 protein, according to SEQ ID No.: 1, in the treatment of a subject having an ocular wound or fibrosis, the effect of its concentration on myofibroblasts was examined. Human corneal keratocytes were incubated for 24 hours in DMEM growth medium with 10% fetal bovine serum (FCS, fetal calf serum) and the human rhC5L2 protein in different concentrations (FIG. 18). For the detection of activated myofibroblasts aSMA antibodies were used, as well as vimentin antibodies as marker for extracellular matrix. As a reference group, human corneal keratocytes incubated for 24 hours in DMEM growth medium with and without fetal bovine serum respectively were used (serumfree: 11±14%; FCS: 20±19%). As shown in FIG. 18, the human rhC5L2 protein was found to have a positive effect on myofibroblasts activation in all concentrations (rhC5L2 0.1 μg/ml: 33±22%; rhC5L2 0.2 μg/ml: 20±24%; rhC5L2 0.3 μg/ml: 41±30%; rhC5L2 0.5 μg/ml: 48±33%). Compared to 10% FCS incubated human corneal keratocytes, differences were significant at rhC5L2 concentrations of 0.1 μg/ml and 0.5 μg/ml (p=0.046, p=0.118, p=0.070 and p=0.033, respectively).

### Example 19

The Effect of the Full-Length Recombinant Human C5a Anaphylatoxin Chemotactic Receptor 2 (rhC5AR2/rhC5L2) Protein Concentration on Myofibroblasts without Fetal Bovine Serum

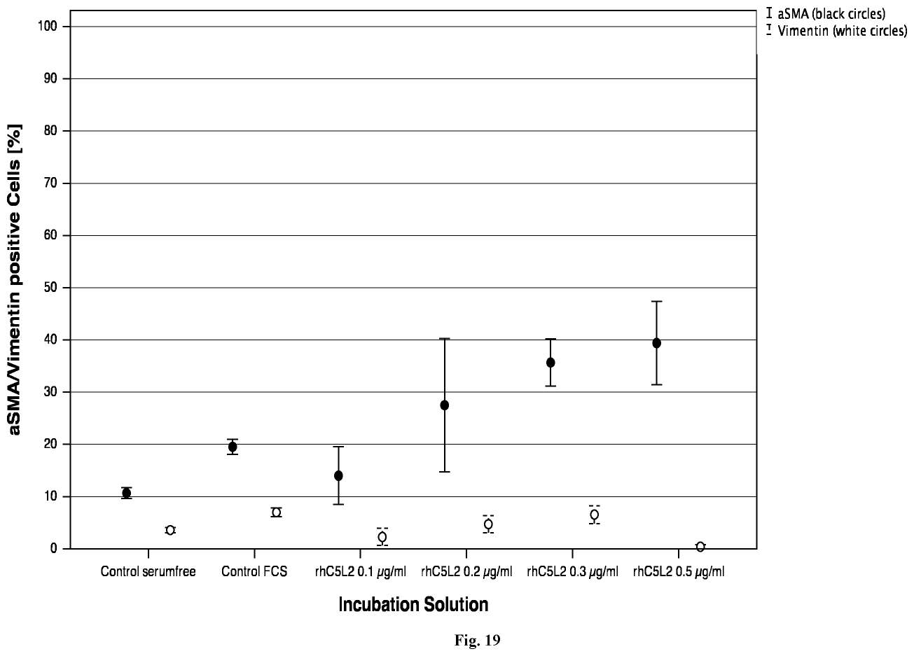

To explore the potential functional role of the human rhC5L2 protein, according to SEQ ID No.: 1, in the treatment of a subject having an ocular wound or fibrosis, the effect of its concentration on myofibroblasts was examined. Human corneal keratocytes were incubated for 24 hours in DMEM growth medium without fetal bovine serum and with the human rhC5L2 protein in different concentrations (FIG. 19). For the detection of activated myofibroblasts aSMA antibodies were used, as well as vimentin antibodies as marker for extracellular matrix. As a reference group, human corneal keratocytes incubated for 24 hours in DMEM growth medium with and without 10% fetal bovine serum (FCS, fetal calf serum) respectively were used (serumfree: 11±14%; FCS: 20±19%). As shown in FIG. 19, the human rhC5L2 protein was found to have a positive effect on myofibroblasts activation, compared to DMEM without FCS incubated human corneal keratocytes (serumfree control), in all concentrations (rhC5L2 0.1 μg/ml: 14±17%; rhC5L2 0.2 μg/ml: 28±38%; rhC5L2 0.3 μg/ml: 36±14%; rhC5L2 0.5 μg/ml: 39±24%). Compared to serumfree control human corneal keratocytes, differences were significant at rhC5L2 concentrations of 0.3 μg/ml and 0.5 μg/ml (p=0.501, p=0.224, p<0.001 and p=0.007, respectively).

### Example 20

Full-Length Recombinant Human C5a Anaphylatoxin Chemotactic Receptor 1 (rhC5AR1) Protein Causes Inhibition of Myofibroblasts Activated by C3a

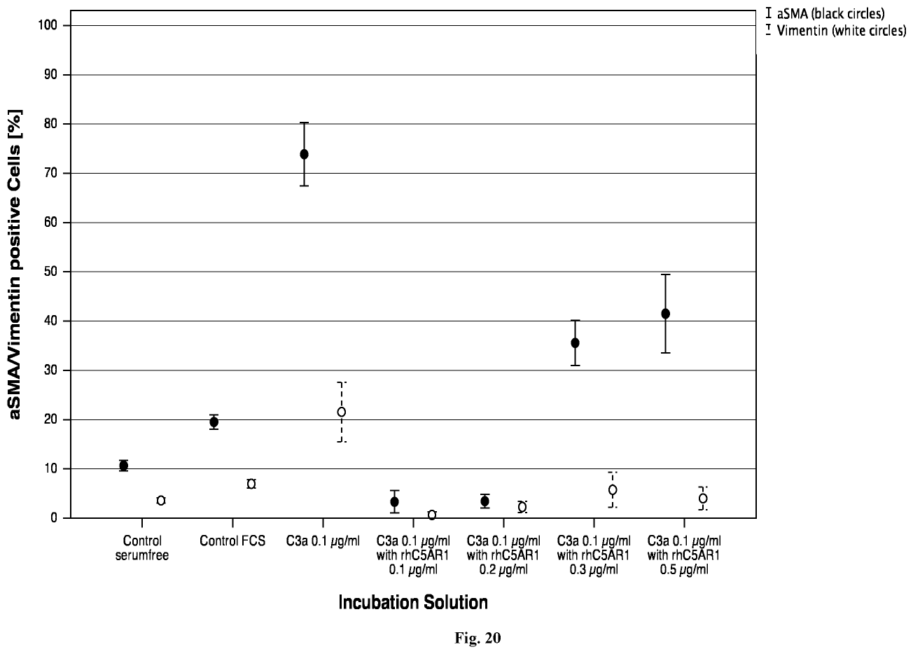

To explore the potential functional role of the rhC5AR1 protein, according to SEQ ID No.: 2, in the treatment of a subject having an ocular wound or fibrosis, the effect of its presence on C3a-activated myofibroblasts was examined. Human corneal keratocytes were stimulated with human C3a for 24 hours and assessed in regard to activated myofibroblasts (FIG. 20). For the detection of activated myofibroblasts aSMA antibodies were used, as well as vimentin antibodies as a marker for extracellular matrix. As a reference group, human corneal keratocytes incubated for 24 hours in DMEM growth medium with and without 10% fetal bovine serum (FCS, fetal calf serum) respectively were used. As shown in FIG. 20, C3a 0.1 μg/ml caused significant activation of myofibroblasts (measured by aSMA positive cells, 74±22%) in comparison with the reference group (serumfree: 11±14%; FCS: 20±19%; p<0.001 and p<0.001, respectively). Incubation in the presence of human C3a and the human rhC5AR1 protein resulted in significant decrease (rhC5AR1 0.1 μg/ml: 3±7%; rhC5AR1 0.2 μg/ml: 3±4%; rhC5AR1 0.3 μg/ml: 36±14%; rhC5AR1 0.5 μg/ml: 42±24%), compared to C3a-activated myofibroblasts (p<0.001, p<0.001, p<0.001 and p=0.005, respectively). Thus, the rhC5AR1 protein was responsible for causing inhibition of myofibroblasts activated by C3a. Bar=Standard error of the mean.

### Example 21

Full-Length Recombinant Human C5a Anaphylatoxin Chemotactic Receptor 1 (rhC5AR1) Protein Causes Inhibition of Myofibroblasts Activated by C5a

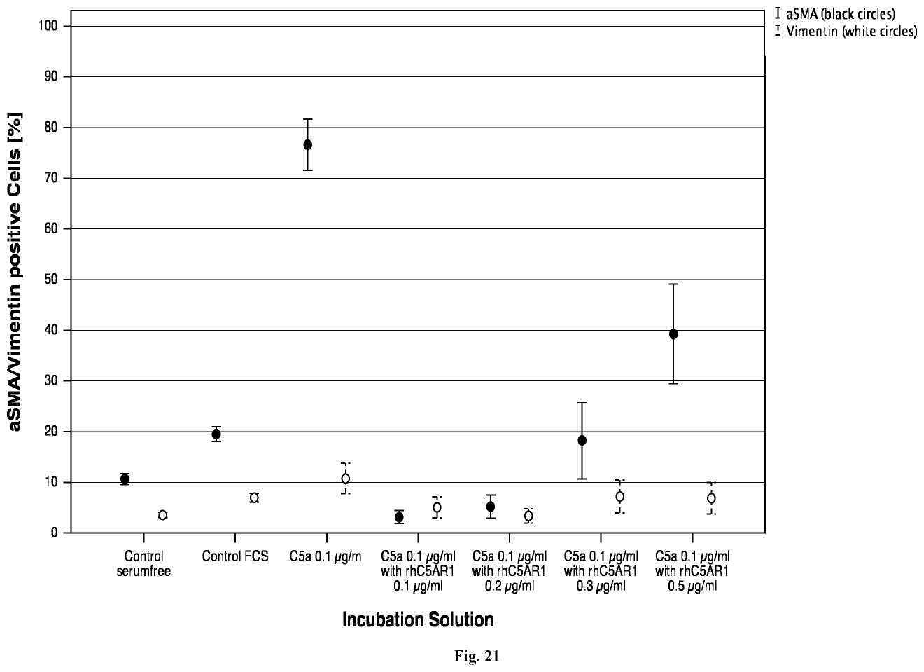

To explore the potential functional role of the rhC5AR1 protein, according to SEQ ID No.: 2, in the treatment of a subject having an ocular wound or fibrosis, the effect of its presence on C5a-activated myofibroblasts was examined. Human corneal keratocytes were stimulated with human C5a for 24 hours and assessed in regard to activated myofibroblasts (FIG. 21). For the detection of activated myofibroblasts aSMA antibodies were used, as well as vimentin antibodies as a marker for extracellular matrix. As a reference group, human corneal keratocytes incubated for 24 hours in DMEM growth medium with and without 10% fetal bovine serum (FCS, fetal calf serum) respectively were used. As shown in FIG. 21, C5a 0.1 μg/ml caused significant activation of myofibroblasts (measured by aSMA positive cells, 77±23%) in comparison with the reference group (serumfree: 11±14%; FCS: 20±19%; p<0.001 and p<0.001, respectively). Incubation in the presence of human C5a and the human rhC5AR1 protein resulted in significant decrease (rhC5AR1 0.1 μg/ml: 3±4%; rhC5AR1 0.2 μg/ml: 5±7%; rhC5AR1 0.3 μg/ml: 18±23%; rhC5AR1 0.5 μg/ml: 39±29%), compared to C5a-activated myofibroblasts (p<0.001, p<0.001, p<0.001 and p=0.001, respectively). Thus, the rhC5AR1 protein was responsible for causing inhibition of myofibroblasts activated by C5a. Bar=Standard error of the mean.

### Example 22

Full-Length Recombinant Human C5a Anaphylatoxin Chemotactic Receptor 1 (rhC5AR1) Protein Causes Inhibition of Myofibroblasts Activated by C5a and C3a

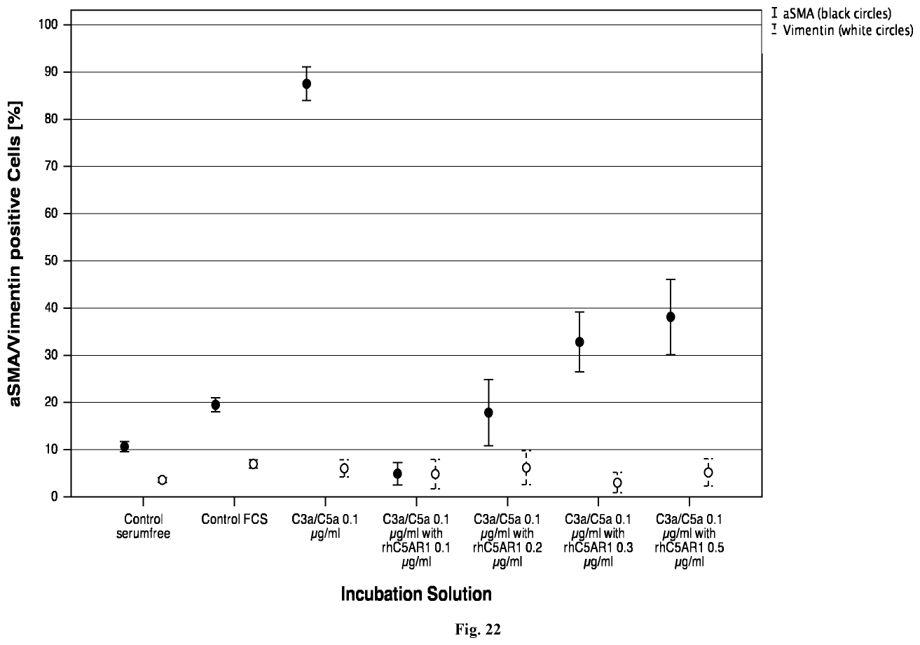

To explore the potential functional role of the rhC5AR1 protein, according to SEQ ID No.: 2, in the treatment of a subject having an ocular wound or fibrosis, the effect of its presence on C5a/C3a-activated myofibroblasts was examined. Human corneal keratocytes were stimulated with human C5a and human C3a for 24 hours and assessed in regard to activated myofibroblasts (FIG. 22). For the detection of activated myofibroblasts aSMA antibodies were used, as well as vimentin antibodies as a marker for extracellular matrix. As a reference group, human corneal keratocytes incubated for 24 hours in DMEM growth medium with and without 10% fetal bovine serum (FCS, fetal calf serum) respectively were used. As shown in FIG. 22, C5a and C3a, both at a concentration of 0.1 μg/ml, caused significant activation of myofibroblasts (measured by aSMA positive cells, 88±11%) in comparison with the reference group (serumfree: 11±14%; FCS: 20±19%; p<0.001 and p<0.001, respectively). Incubation in the presence of human C3a, C5a and the human rhC5AR1 protein resulted in significant decrease (rhC5AR1 0.1 μg/ml: 5±7%; rhC5AR1 0.2 μg/ml: 18±21%; rhC5AR1 0.3 μg/ml: 33±19%; rhC5AR1 0.5 μg/ml: 38±24%), compared to C5a and C3a-activated myofibroblasts (p<0.001, p<0.001, p<0.001 and p<0.001, respectively). Thus, the rhC5AR1 protein was responsible for causing inhibition of myofibroblasts activated by C5a and C3a. Bar=Standard error of the mean.

### Example 23

The Effect of the Full-Length Recombinant Human C5a Anaphylatoxin Chemotactic Receptor 1 (rhC5AR1) Protein Concentration on Myofibroblasts in the Presence of Fetal Bovine Serum

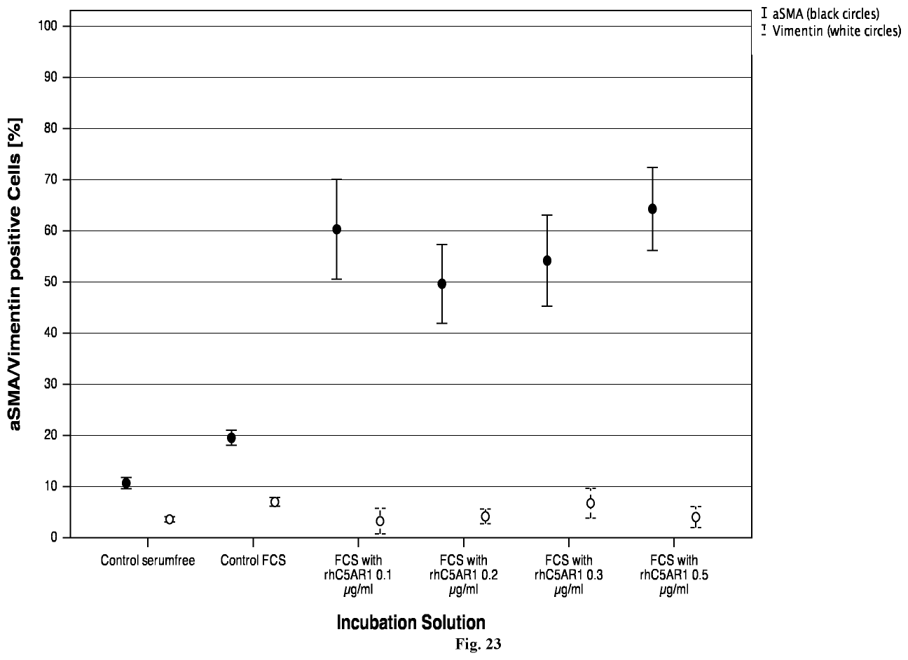

To explore the potential functional role of the human rhC5AR1 protein, according to SEQ ID No.: 2, in the treatment of a subject having an ocular wound or fibrosis, the effect of its concentration on myofibroblasts was examined. Human corneal keratocytes were incubated for 24 hours in DMEM growth medium with 10% fetal bovine serum (FCS, fetal calf serum) and the human rhC5AR1 protein in different concentrations (FIG. 23). For the detection of activated myofibroblasts aSMA antibodies were used, as well as vimentin antibodies as marker for extracellular matrix. As a reference group, human corneal keratocytes incubated for 24 hours in DMEM growth medium with and without fetal bovine serum respectively were used (serumfree: 11±14%; FCS: 20±19%). As shown in FIG. 23, the human rhC5AR1 protein was found to have a positive effect on myofibroblasts activation in all concentrations (rhC5AR1 0.1 μg/ml: 60±29%; rhC5AR1 0.2 μg/ml: 50±23%; rhC5AR1 0.3 μg/ml: 54±27%; rhC5AR1 0.5 μg/ml: 64±24%). Compared to 10% FCS incubated human corneal keratocytes, differences were significant (p=0.003, p<0.001, p<0.001 and p<0.001, respectively).

### Example 24

The Effect of the Full-Length Recombinant Human C5a Anaphylatoxin Chemotactic Receptor 1 (rhC5AR1) Protein Concentration on Myofibroblasts without Fetal Bovine Serum

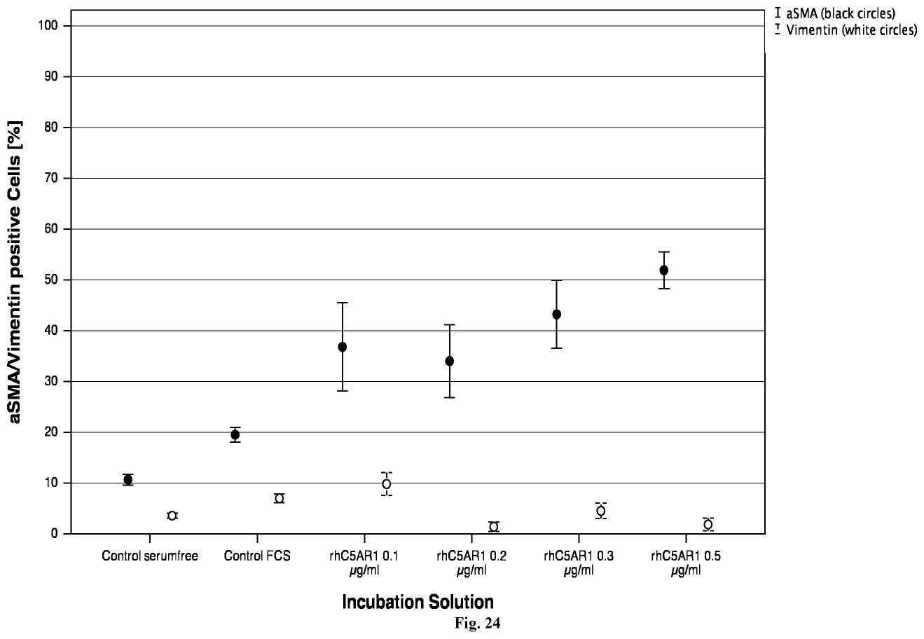

To explore the potential functional role of the human rhC5AR1 protein, according to SEQ ID No.: 2, in the treatment of a subject having an ocular wound or fibrosis, the effect of its concentration on myofibroblasts was examined. Human corneal keratocytes were incubated for 24 hours in DMEM growth medium without fetal bovine serum and with the human rhC5AR1 protein in different concentrations (FIG. 24). For the detection of activated myofibroblasts aSMA antibodies were used, as well as vimentin antibodies as marker for extracellular matrix. As a reference group, human corneal keratocytes incubated for 24 hours in DMEM growth medium with and without 10% fetal bovine serum (FCS, fetal calf serum) respectively were used (serumfree: 11±14%; FCS: 20±19%). As shown in FIG. 24, the human rhC5AR1 protein was found to have a positive effect on myofibroblasts activation, compared to DMEM without FCS incubated human corneal keratocytes (serumfree control), in all concentrations (rhC5AR1 0.1 μg/ml: 37±26%; rhC5AR1 0.2 μg/ml: 34±22%; rhC5AR1 0.3 μg/ml: 43±20%; rhC5AR1 0.5 μg/ml: 52±11%). Compared to serumfree control human corneal keratocytes, differences were significant (p=0.017, p=0.012, p<0.001 and p<0.001, respectively).

### Example 25

Full-Length Recombinant Human C3a Anaphylatoxin Chemotactic Receptor (rhC3AR) Protein Causes Inhibition of Myofibroblasts Activated by C3a

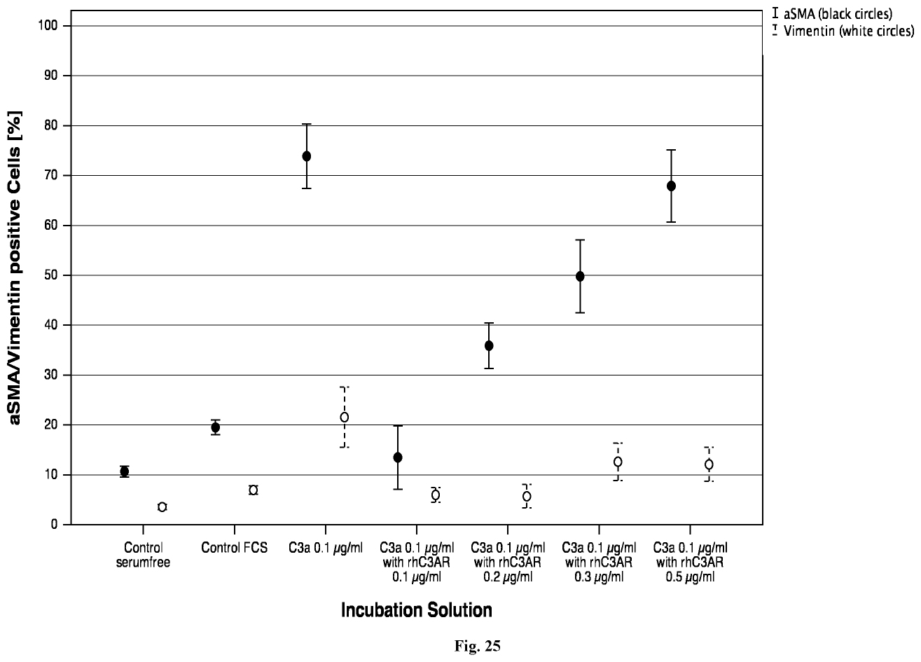

To explore the potential functional role of the rhC3AR protein, according to SEQ ID No.: 3, in the treatment of a subject having an ocular wound or fibrosis, the effect of its presence on C3a-activated myofibroblasts was examined. Human corneal keratocytes were stimulated with human C3a for 24 hours and assessed in regard to activated myofibroblasts (FIG. 25). For the detection of activated myofibroblasts aSMA antibodies were used, as well as vimentin antibodies as a marker for extracellular matrix. As a reference group, human corneal keratocytes incubated for 24 hours in DMEM growth medium with and without 10% fetal bovine serum (FCS, fetal calf serum) respectively were used. As shown in FIG. 25, C3a 0.1 μg/ml caused significant activation of myofibroblasts (measured by aSMA positive cells, 74±22%) in comparison with the reference group (serumfree: 11±14%; FCS: 20±19%; p<0.001 and p<0.001, respectively). Incubation in the presence of human C3a and the human rhC3AR protein resulted in significant decrease (rhC3AR 0.1 μg/ml: 13±19%; rhC3AR 0.2 μg/ml: 36±14%; rhC3AR 0.3 μg/ml: 50±22%; rhC3AR 0.5 μg/ml: 68±22%), compared to C3a-activated myofibroblasts (p<0.001, p<0.001, p=0.023 and p=0.547, respectively). Thus, the rhC3AR protein in concentrations of 0.1 μg/ml, 0.2 μg/ml and 0.3 μg/ml was responsible for causing inhibition of myofibroblasts activated by C3a. Bar=Standard error of the mean.

### Example 26

Full-Length Recombinant Human C3a Anaphylatoxin Chemotactic Receptor (rhC3AR) Protein Causes Inhibition of Myofibroblasts Activated by C5a

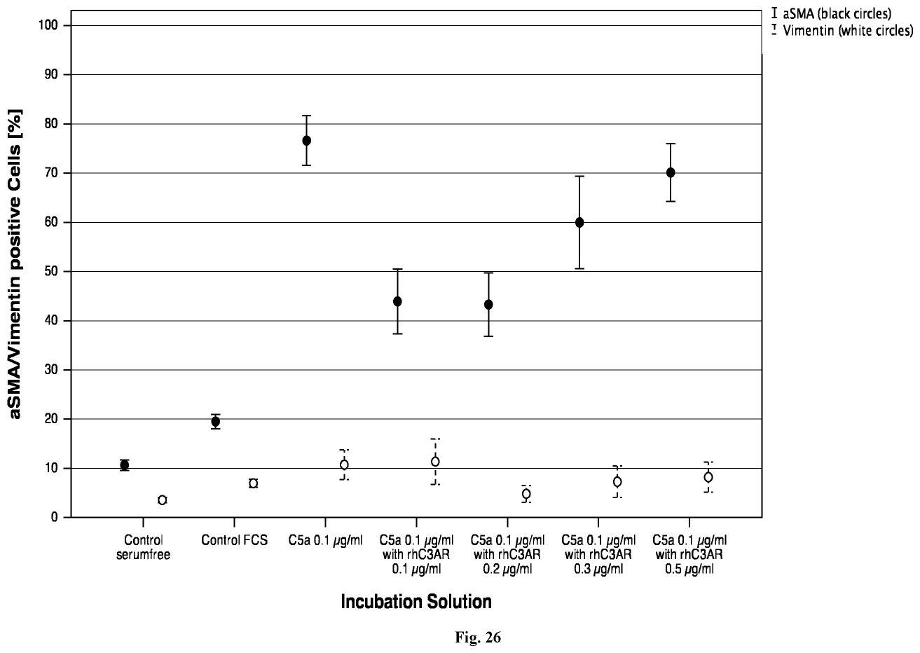

To explore the potential functional role of the rhC3AR protein, according to SEQ ID No.: 3, in the treatment of a subject having an ocular wound or fibrosis, the effect of its presence on C5a-activated myofibroblasts was examined. Human corneal keratocytes were stimulated with human C5a for 24 hours and assessed in regard to activated myofibroblasts (FIG. 26). For the detection of activated myofibroblasts aSMA antibodies were used, as well as vimentin antibodies as a marker for extracellular matrix. As a reference group, human corneal keratocytes incubated for 24 hours in DMEM growth medium with and without 10% fetal bovine serum (FCS, fetal calf serum) respectively were used. As shown in FIG. 26, C5a 0.1 μg/ml caused significant activation of myofibroblasts (measured by aSMA positive cells, 77±23%) in comparison with the reference group (serumfree: 11±14%; FCS: 20±19%; p<0.001 and p<0.001, respectively). Incubation in the presence of human C5a and the human rhC3AR protein resulted in significant decrease (rhC3AR 0.1 μg/ml: 44±20%; rhC3AR 0.2 μg/ml: 43±19%; rhC3AR 0.3 μg/ml: 60±28%; rhC3AR 0.5 μg/ml: 70±18%), compared to C5a-activated myofibroblasts (p=0.001, p=0.001, p=0.103 and p=0.460, respectively). Thus, the rhC3AR protein in concentrations of 0.1 μg/ml and 0.2 μg/ml was responsible for causing inhibition of myofibroblasts activated by C5a. Bar=Standard error of the mean.

### Example 27

Full-Length Recombinant Human C3a Anaphylatoxin Chemotactic Receptor (rhC3AR) Protein Causes Inhibition of Myofibroblasts Activated by C5a and C3a

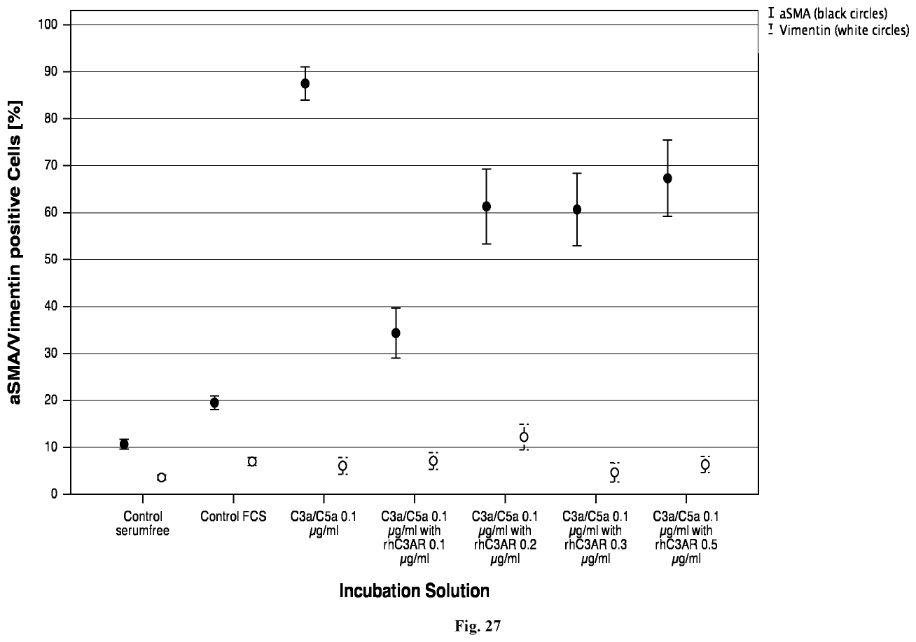

To explore the potential functional role of the rhC3AR protein, according to SEQ ID No.: 3, in the treatment of a subject having an ocular wound or fibrosis, the effect of its presence on C5a/C3a-activated myofibroblasts was examined. Human corneal keratocytes were stimulated with human C5a and human C3a for 24 hours and assessed in regard to activated myofibroblasts (FIG. 27). For the detection of activated myofibroblasts aSMA antibodies were used, as well as vimentin antibodies as a marker for extracellular matrix. As a reference group, human corneal keratocytes incubated for 24 hours in DMEM growth medium with and without 10% fetal bovine serum (FCS, fetal calf serum) respectively were used. As shown in FIG. 27, C5a and C3a, both at a concentration of 0.1 μg/ml, caused significant activation of myofibroblasts (measured by aSMA positive cells, 88±11%) in comparison with the reference group (serumfree: 11±14%; FCS: 20±19%; p<0.001 and p<0.001, respectively). Incubation in the presence of human C3a, C5a and the human rhC3AR protein resulted in significant decrease (rhC3AR 0.1 μg/ml: 34±16%; rhC3AR 0.2 μg/ml: 61±24%; rhC3AR 0.3 μg/ml: 61±23%; rhC3AR 0.5 μg/ml: 67±24%), compared to C5a and C3a-activated myofibroblasts (p<0.001, p=0.012, p=0.006 and p=0.044, respectively). Thus, the rhC3AR protein was responsible for causing inhibition of myofibroblasts activated by C5a and C3a. Bar=Standard error of the mean.

### Example 28

The Effect of the Full-Length Recombinant Human C3a Anaphylatoxin Chemotactic Receptor (rhC3AR) Protein Concentration on Myofibroblasts in the Presence of Fetal Bovine Serum

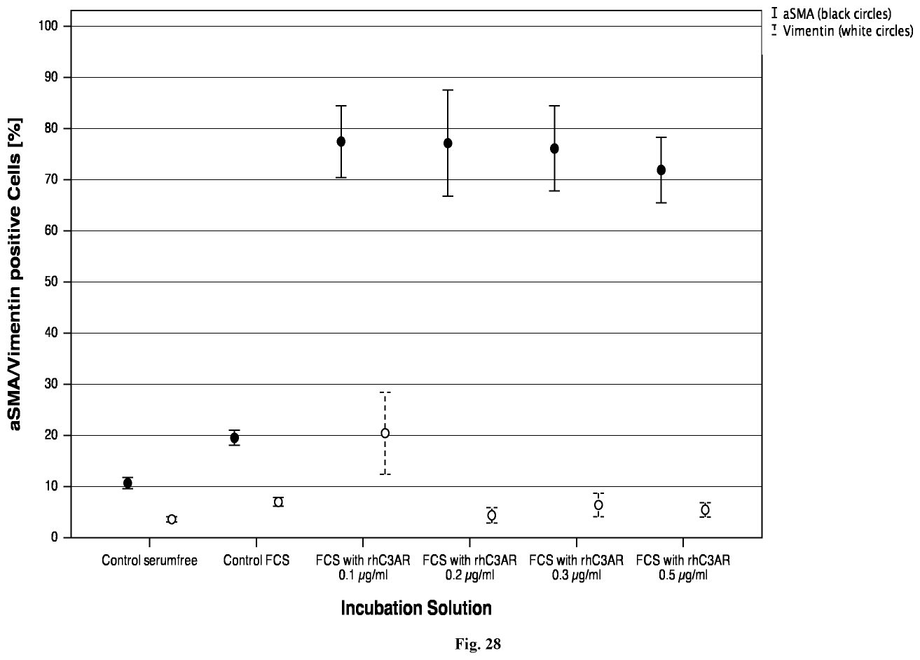

To explore the potential functional role of the human rhC3AR protein, according to SEQ ID No.: 3, in the treatment of a subject having an ocular wound or fibrosis, the effect of its concentration on myofibroblasts was examined. Human corneal keratocytes were incubated for 24 hours in DMEM growth medium with 10% fetal bovine serum (FCS, fetal calf serum) and the human rhC3AR protein in different concentrations (FIG. 28). For the detection of activated myofibroblasts aSMA antibodies were used, as well as vimentin antibodies as marker for extracellular matrix. As a reference group, human corneal keratocytes incubated for 24 hours in DMEM growth medium with and without fetal bovine serum respectively were used (serumfree: 11±14%; FCS: 20±19%). As shown in FIG. 28, the human rhC3AR protein was found to have a positive effect on myofibroblasts activation in all concentrations (rhC3AR 0.1 μg/ml: 77±21%; rhC3AR 0.2 μg/ml: 77±31%; rhC3AR 0.3 μg/ml: 76±25%; rhC3AR 0.5 μg/ml: 72±19%). Compared to 10% FCS incubated human corneal keratocytes, differences were significant (p<0.001, p<0.001, p<0.001 and p<0.001, respectively).

### Example 29

The Effect of the Full-Length Recombinant Human C3a Anaphylatoxin Chemotactic Receptor (rhC3AR) Protein Concentration on Myofibroblasts without Fetal Bovine Serum

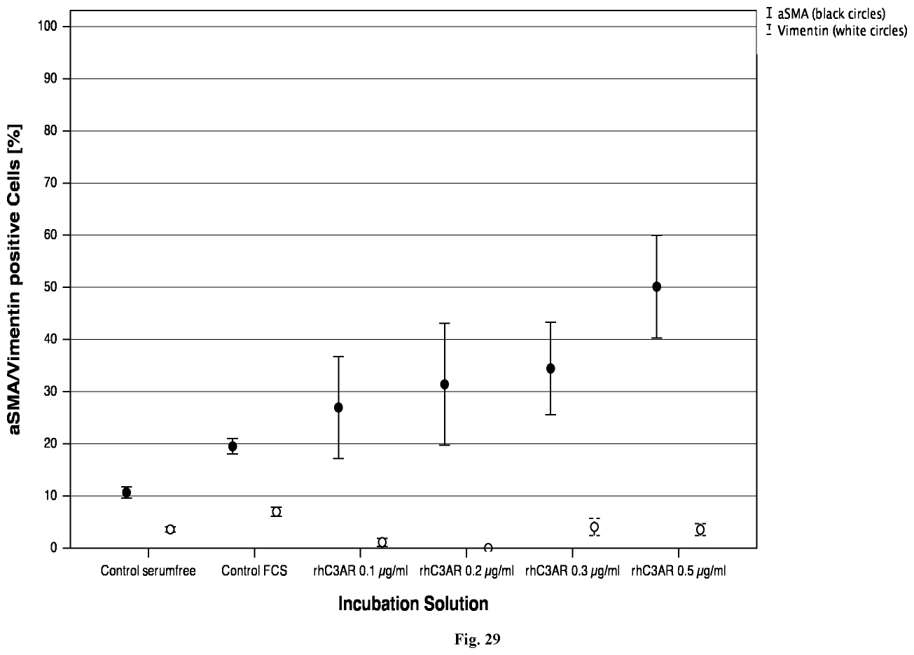

To explore the potential functional role of the human rhC3AR protein, according to SEQ ID No.: 3, in the treatment of a subject having an ocular wound or fibrosis, the effect of its concentration on myofibroblasts was examined. Human corneal keratocytes were incubated for 24 hours in DMEM growth medium without fetal bovine serum and with the human rhC3AR protein in different concentrations (FIG. 29). For the detection of activated myofibroblasts aSMA antibodies were used, as well as vimentin antibodies as marker for extracellular matrix. As a reference group, human corneal keratocytes incubated for 24 hours in DMEM growth medium with and without 10% fetal bovine serum (FCS, fetal calf serum) respectively were used (serumfree: 11±14%; FCS: 20±19%). As shown in FIG. 29, the human rhC3AR protein was found to have a positive effect on myofibroblasts activation, compared to DMEM without FCS incubated human corneal keratocytes (serumfree control), in all concentrations (rhC3AR 0.1 μg/ml: 27±29%; rhC3AR 0.2 μg/ml: 31±35%; rhC3AR 0.3 μg/ml: 34±27%; rhC3AR 0.5 μg/ml: 50±29%). Compared to serumfree control human corneal keratocytes, differences were significant at rhC3AR concentrations of 0.3 μg/ml and 0.5 μg/ml (p=0.136, p=0.114, p=0.028 and p=0.004, respectively).

### Example 30

RNA/DNA Aptamer Binding to Human C5a Causes Inhibition of Myofibroblasts Activated by C3a

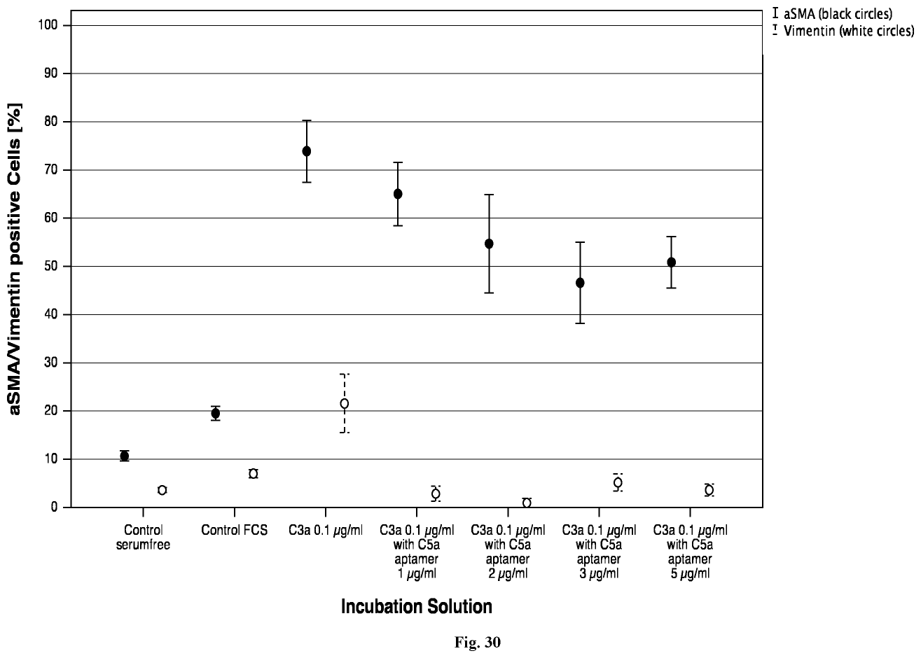

To explore the potential functional role of the L-RNA/L-DNA aptamer binding to human C5a (C5a aptamer), containing a C5a binding site according to SEQ ID No.: 41, in the treatment of a subject having an ocular wound or fibrosis, the effect of its presence on C3a-activated myofibroblasts was examined. Human corneal keratocytes were stimulated with human C3a for 24 hours and assessed in regard to activated myofibroblasts (FIG. 30). For the detection of activated myofibroblasts aSMA antibodies were used, as well as vimentin antibodies as a marker for extracellular matrix. As a reference group, human corneal keratocytes incubated for 24 hours in DMEM growth medium with and without 10% fetal bovine serum (FCS, fetal calf serum) respectively were used. As shown in FIG. 30, C3a 0.1 μg/ml caused significant activation of myofibroblasts (measured by aSMA positive cells, 74±22%) in comparison with the reference group (serumfree: 11±14%; FCS: 20±19%; p<0.001 and p<0.001, respectively). Incubation in the presence of human C3a and the C5a aptamer resulted in significant decrease (C5a aptamer 1 μg/ml: 65±20%; C5a aptamer 2 μg/ml: 55±31%; C5a aptamer 3 μg/ml: 47±25%; C5a aptamer 5 μg/ml: 51±16%), compared to C3a-activated myofibroblasts (p=0.356, p=0.112, p=0.017 and p=0.017, respectively). Thus, the C5a aptamer in concentrations of 3 μg/ml and 5 μg/ml was responsible for causing inhibition of myofibroblasts activated by C3a. Bar=Standard error of the mean.

### Example 31

RNA/DNA Aptamer Binding to Human C5a Causes Inhibition of Myofibroblasts Activated by C5a

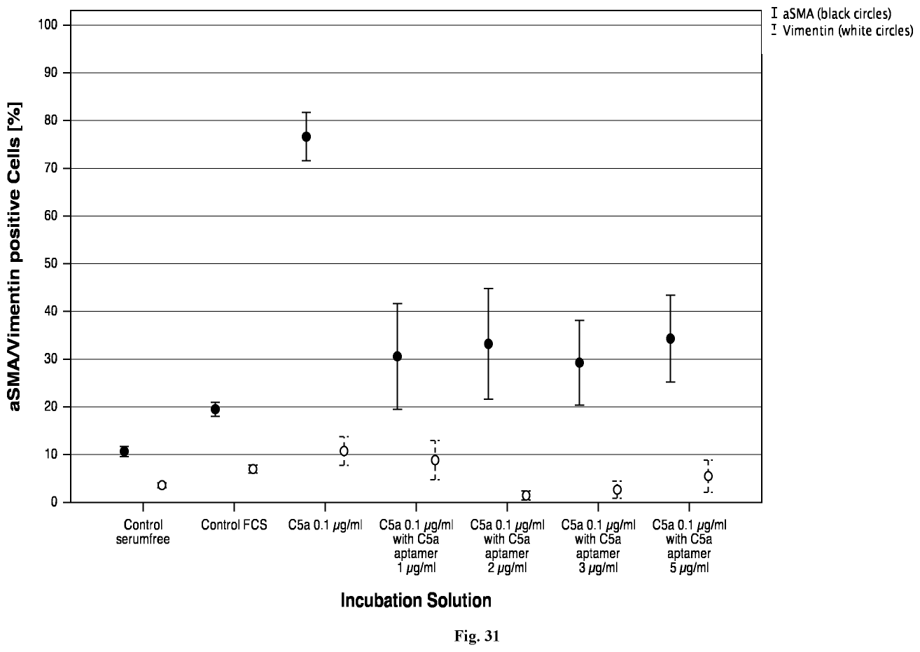

To explore the potential functional role of the L-RNA/L-DNA aptamer binding to human C5a (C5a aptamer), containing a C5a binding site according to SEQ ID No.: 41, in the treatment of a subject having an ocular wound or fibrosis, the effect of its presence on C5a-activated myofibroblasts was examined. Human corneal keratocytes were stimulated with human C5a for 24 hours and assessed in regard to activated myofibroblasts (FIG. 31). For the detection of activated myofibroblasts aSMA antibodies were used, as well as vimentin antibodies as a marker for extracellular matrix. As a reference group, human corneal keratocytes incubated for 24 hours in DMEM growth medium with and without 10% fetal bovine serum (FCS, fetal calf serum) respectively were used. As shown in FIG. 31, C5a 0.1 μg/ml caused significant activation of myofibroblasts (measured by aSMA positive cells, 77±23%) in comparison with the reference group (serumfree: 11±14%; FCS: 20±19%; p<0.001 and p<0.001, respectively). Incubation in the presence of human C5a and the C5a aptamer resulted in significant decrease (C5a aptamer 1 μg/ml: 31±33%; C5a aptamer 2 μg/ml: 33±35%; C5a aptamer 3 μg/ml: 29±27%; C5a aptamer 5 μg/ml: 34±27%), compared to C5a-activated myofibroblasts (p<0.001, p<0.001, p<0.001 and p<0.001, respectively). Thus, the C5a aptamer was responsible for causing inhibition of myofibroblasts activated by C5a. Bar=Standard error of the mean.

### Example 32

RNA/DNA Aptamer Binding to Human C5a Causes Inhibition of Myofibroblasts Activated by C5a and C3a

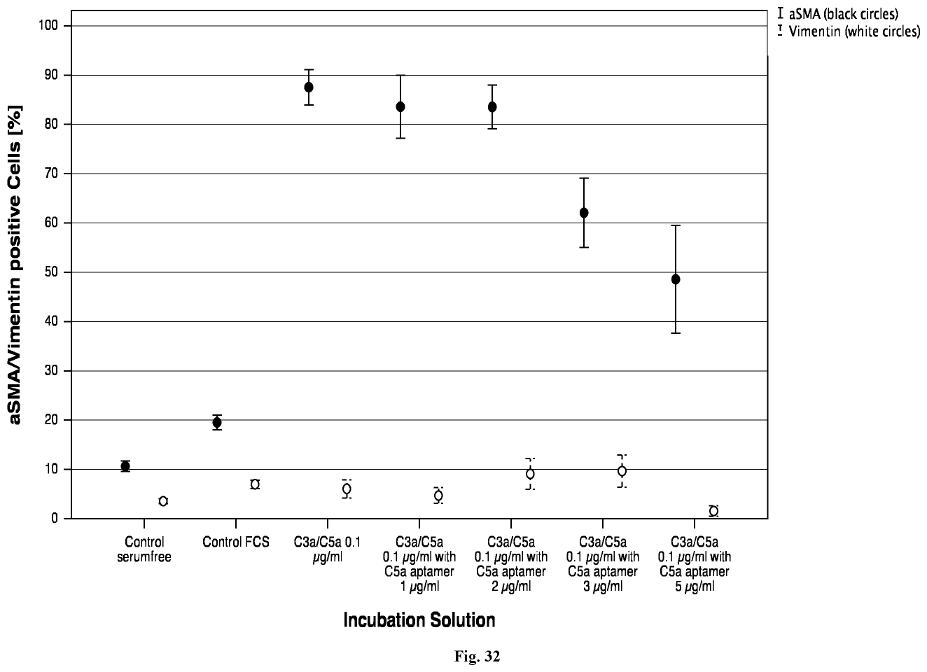

To explore the potential functional role of the L-RNA/L-DNA aptamer binding to human C5a (C5a aptamer), containing a C5a binding site according to SEQ ID No.: 41, in the treatment of a subject having an ocular wound or fibrosis, the effect of its presence on C5a/C3a-activated myofibroblasts was examined. Human corneal keratocytes were stimulated with human C5a and human C3a for 24 hours and assessed in regard to activated myofibroblasts (FIG. 32). For the detection of activated myofibroblasts aSMA antibodies were used, as well as vimentin antibodies as a marker for extracellular matrix. As a reference group, human corneal keratocytes incubated for 24 hours in DMEM growth medium with and without 10% fetal bovine serum (FCS, fetal calf serum) respectively were used. As shown in FIG. 32, C5a and C3a, both at a concentration of 0.1 μg/ml, caused significant activation of myofibroblasts (measured by aSMA positive cells, 88±11%) in comparison with the reference group (serumfree: 11±14%; FCS: 20±19%; p<0.001 and p<0.001, respectively). Incubation in the presence of C3a, C5a and the C5a aptamer resulted in significant decrease (C5a aptamer 1 μg/ml: 84±13%; C5a aptamer 2 μg/ml: 84±13%; C5a aptamer 3 μg/ml: 62±21%; C5a aptamer 5 μg/ml: 49±33%), compared to C5a and C3a-activated myofibroblasts (p=0.519, p=0.495, p=0.005 and p=0.007, respectively). Thus, the C5a aptamer was responsible for causing inhibition of myofibroblasts activated by C5a and C3a. Bar=Standard error of the mean.

### Example 33

The Effect of the RNA/DNA Aptamer, Binding to Human C5a, Concentration on Myofibroblasts in the Presence of Fetal Bovine Serum

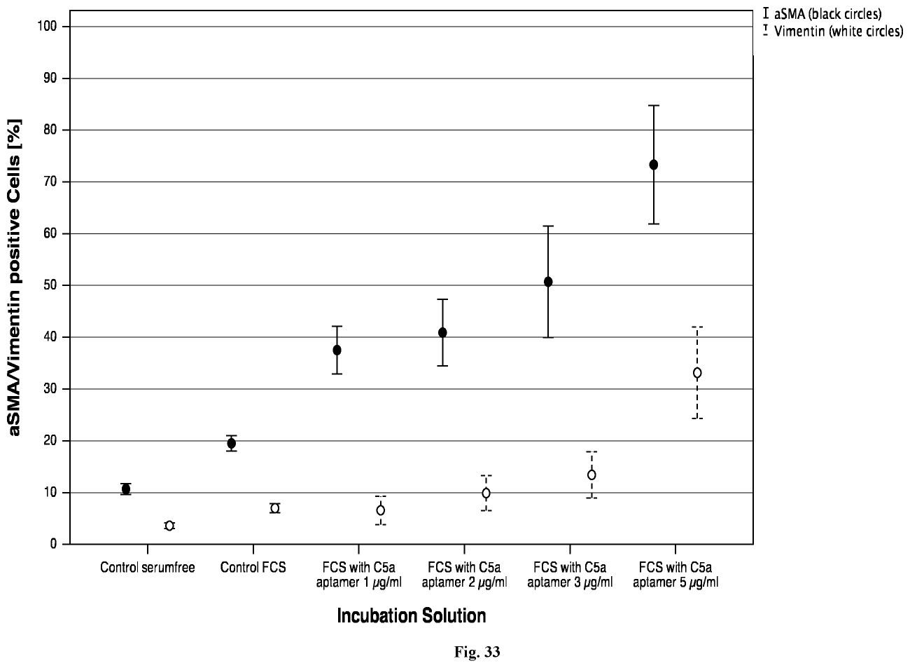

To explore the potential functional role of the L-RNA/L-DNA aptamer binding to human C5a (C5a aptamer), containing a C5a binding site according to SEQ ID No.: 41, in the treatment of a subject having an ocular wound or fibrosis, the effect of its concentration on myofibroblasts was examined. Human corneal keratocytes were incubated for 24 hours in DMEM growth medium with 10% fetal bovine serum (FCS, fetal calf serum) and the C5a aptamer in different concentrations (FIG. 33). For the detection of activated myofibroblasts aSMA antibodies were used, as well as vimentin antibodies as marker for extracellular matrix. As a reference group, human corneal keratocytes incubated for 24 hours in DMEM growth medium with and without fetal bovine serum respectively were used (serumfree: 11±14%; FCS: 20±19%). As shown in FIG. 33, the C5a aptamer was found to have a positive effect on myofibroblasts activation in all concentrations (C5a aptamer 1 μg/ml: 38±14%; C5a aptamer 2 μg/ml: 41±19%; C5a aptamer 3 μg/ml: 51±32%; C5a aptamer 5 μg/ml: 73±34%). Compared to 10% FCS incubated human corneal keratocytes, differences were significant (p=0.005, p=0.001, p=0.020 and p=0.001, respectively).

### Example 34

The Effect of the RNA/DNA Aptamer, Binding to Human C5a, Concentration on Myofibroblasts without Fetal Bovine Serum

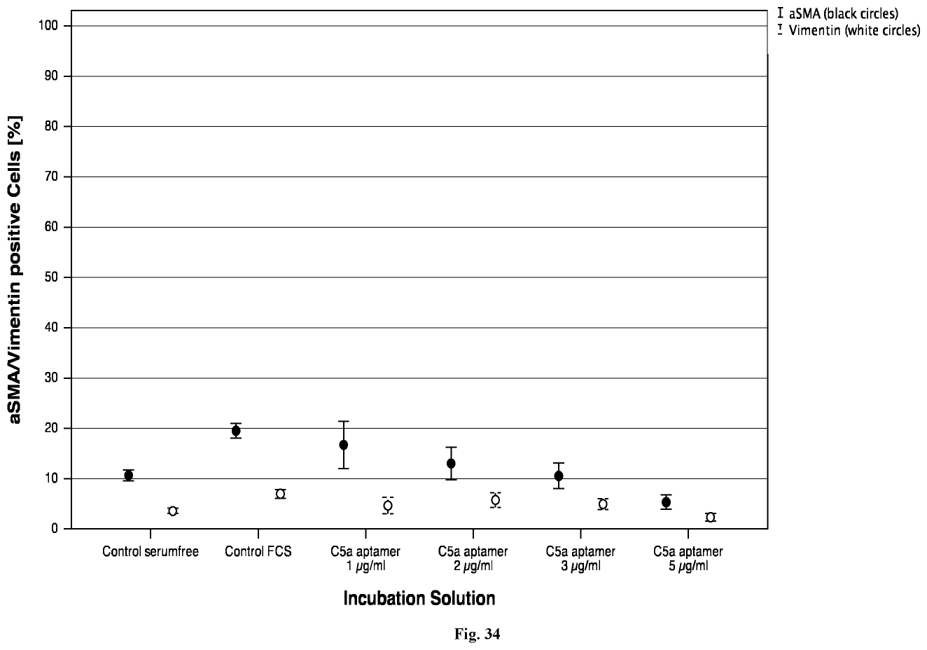

To explore the potential functional role of the L-RNA/L-DNA aptamer binding to human C5a (C5a aptamer), containing a C5a binding site according to SEQ ID No.: 41, in the treatment of a subject having an ocular wound or fibrosis, the effect of its concentration on myofibroblasts was examined. Human corneal keratocytes were incubated for 24 hours in DMEM growth medium without fetal bovine serum and with the C5a aptamer in different concentrations (FIG. 34). For the detection of activated myofibroblasts aSMA antibodies were used, as well as vimentin antibodies as marker for extracellular matrix. As a reference group, human corneal keratocytes incubated for 24 hours in DMEM growth medium with and without 10% fetal bovine serum (FCS, fetal calf serum) respectively were used (serumfree: 11±14%; FCS: 20±19%). As shown in FIG. 34, the C5a aptamer was found to have a slightly positive effect on myofibroblasts activation, compared to DMEM without FCS incubated human corneal keratocytes (serumfree control), in all concentrations (C5a aptamer 1 μg/ml: 17±14%; C5a aptamer 2 μg/ml: 13±10%; C5a aptamer 3 μg/ml: 11±8%; C5a aptamer 5 μg/ml: 5±4%). However, compared to serumfree control human corneal keratocytes, differences were not significant (p=0.219, p=0.629, p=0.983 and p=0.270, respectively).

### Example 35

Antibodies Binding to Human C5a Cause Inhibition of Myofibroblasts Activated by C5a, but do not Cause Inhibition of Myofibroblasts Activated by C3a nor C3a and C5a Combined.

To explore the potential functional role of antibodies binding to human C5a (C5a Ab) in the treatment of a subject having an ocular wound or fibrosis, the effects of its presence on C3a-, C5a- and C5a/C3a-activated myofibroblasts were examined. Furthermore, the effects of its concentrations on myofibroblasts with and without the presence of fetal bovine serum were examined, as well.

The antibodies examined were the polyclonal rabbit immunoglobulin G antibody 250565 (Abbiotec; San Diego, USA), raised against the sequence within amino acids 700-755 of the human complement C5 isoform 1 preproprotein (Accession No.: NP_001726), that corresponds to the sequence within amino acids 23-74 of SEQ ID No.: 20; and the polyclonal rabbit immunoglobulin G antibody 308733 (Biorbyt; Cambridge, United Kingdom), raised against the sequence within amino acids 1275-1290 of the human complement C5 isoform 1 preproprotein (Accession No.: NP_001726).

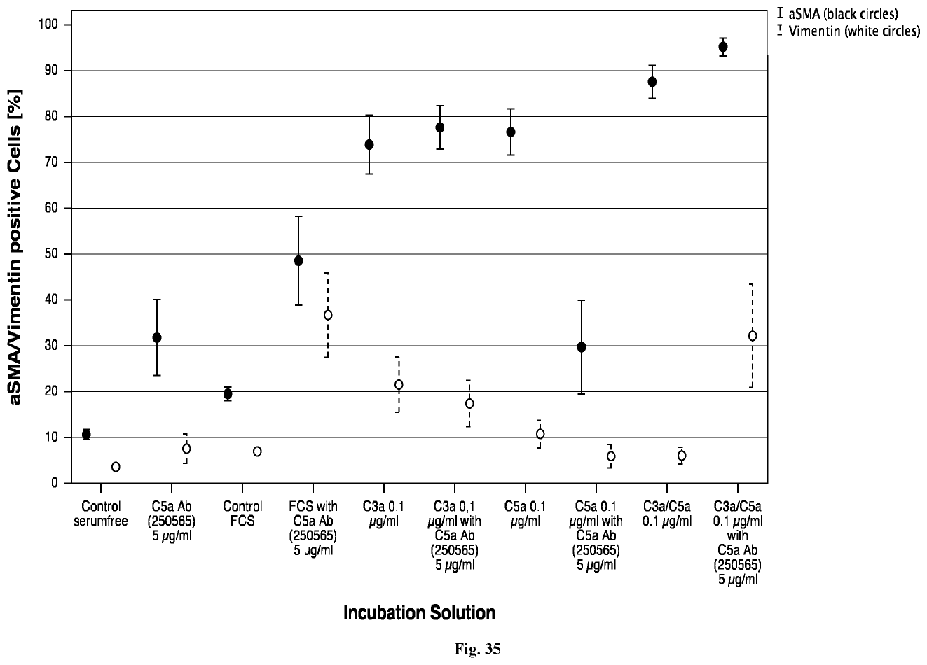

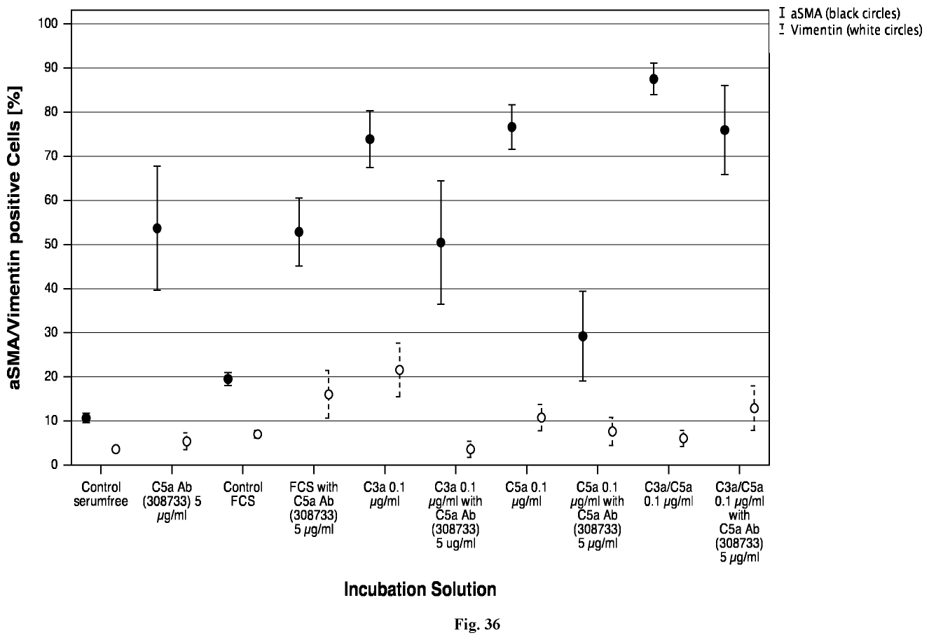

Human corneal keratocytes were stimulated with human C3a, human C5a and human C5a/C3a combined for 24 hours and assessed in regard to activated myofibroblasts (FIGS. 35 and 36). For the detection of activated myofibroblasts aSMA antibodies were used, as well as vimentin antibodies as a marker for extracellular matrix. As a reference group, human corneal keratocytes incubated for 24 hours in DMEM growth medium with and without 10% fetal bovine serum (FCS, fetal calf serum) respectively were used. As shown in FIGS. 35 and 36, C3a, C5a and C5a/C3a caused significant activation of myofibroblasts (measured by aSMA positive cells; C3a 0.1 μg/ml: 74±22%; C5a 0.1 μg/ml: 77±23%; C5a 0.1 μg/ml and C3a 0.1 μg/ml: 88±11%) in comparison with the reference group (serumfree: 11±14%; FCS: 20±19%; p-values<0.001). Incubation in the presence of human C3a and C5a antibodies resulted in no significant decrease (C5a Ab (250565) 5 μg/ml: 78±14%; C5a Ab (308733) 5 μg/ml: 50±42%), compared to C3a-activated myofibroblasts (p=0.645, p=0.155, respectively). Incubation in the presence of human C5a and C5a antibodies resulted in a significant decrease (C5a Ab (250565) 5 μg/ml: 30±31%; C5a Ab (308733) 5 μg/ml: 29±31%), compared to C5a-activated myofibroblasts (p<0.001, p<0.001, respectively). Incubation in the presence of human C3a, C5a and C5a antibodies resulted in no significant decrease (C5a Ab (250565) 5 μg/ml: 95±6%; C5a Ab (308733) 5 μg/ml: 76±30%), compared to C5a and C3a-activated myofibroblasts (p=0.079, p=0.294, respectively). As shown in FIGS. 35 and 36, the C5a antibodies were found to have a positive effect on myofibroblasts activation in the presence of 10% FCS (C5a Ab (250565) 5 μg/ml: 49±29%; C5a Ab (308733) 5 μg/ml: 53±23%). Compared to 10% FCS incubated human corneal keratocytes, differences were significant (p<0.001, p=0.002, respectively). As shown in FIGS. 35 and 36, the C5a antibodies were found to have a positive effect on myofibroblasts activation, compared to DMEM without FCS incubated human corneal keratocytes (serumfree control), in all concentrations (C5a Ab (250565) 5 μg/ml: 32±25%; C5a Ab (308733) 5 μg/ml: 54±42%). Compared to serumfree control human corneal keratocytes, differences were significant (p=0.034, p=0.016, respectively). Thus, the C5a antibodies 250565 (Abbiotec) and 308733 (Biorbyt) in concentrations of 5 μg/ml were responsible for causing inhibition of myofibroblasts activated by C5a, but not by C3a nor C3a and C5a combined. Bar=Standard error of the mean.

### Example 36

Antibodies Binding to Human C3a Cause Inhibition of Myofibroblasts Activated by C3a, but do not Cause Inhibition of Myofibroblasts Activated by C5a nor C3a and C5a Combined.

To explore the potential functional role of antibodies binding to human C3a (C3a mAb) in the treatment of a subject having an ocular wound or fibrosis, the effects of its presence on C3a-, C5a- and C5a/C3a-activated myofibroblasts were examined. Furthermore, the effects of its concentrations on myofibroblasts with and without the presence of fetal bovine serum were examined, as well.

The antibodies examined were the monoclonal mouse immunoglobulin G₁ (kappa light chain) antibody sc28294 (Santa Cruz Biotechnology; Dallas, USA), raised against the sequence within amino acids 541-840 of the human complement C3 preproprotein (Accession No.: NP_000055.2), that covers SEQ ID No.: 43; and the monoclonal rat immunoglobulin G₂ₐ antibody HM1072 (Hycult Biotech; Uden, The Netherlands), raised against a sequence of the mouse C5 protein (Specification according to the reference by Mastellos D et al. Mol Immunol 2004).

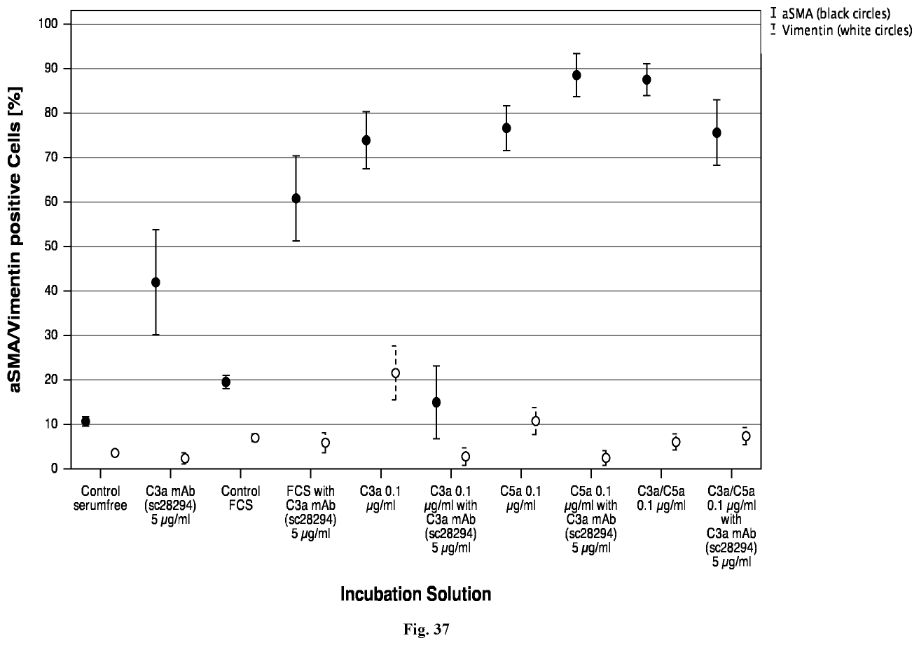

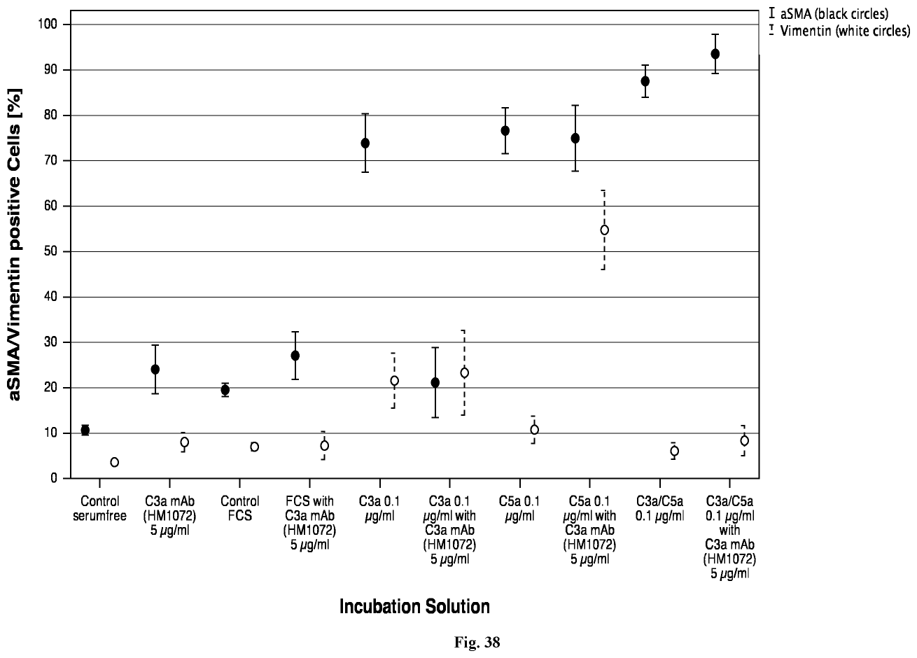

Human corneal keratocytes were stimulated with human C3a, human C5a and human C5a/C3a combined for 24 hours and assessed in regard to activated myofibroblasts (FIGS. 37 and 38). For the detection of activated myofibroblasts aSMA antibodies were used, as well as vimentin antibodies as a marker for extracellular matrix. As a reference group, human corneal keratocytes incubated for 24 hours in DMEM growth medium with and without 10% fetal bovine serum (FCS, fetal calf serum) respectively were used. As shown in FIGS. 37 and 38, C3a, C5a and C5a/C3a caused significant activation of myofibroblasts (measured by aSMA positive cells; C3a 0.1 μg/ml: 74±22%; C5a 0.1 μg/ml: 77±23%; C5a 0.1 μg/ml and C3a 0.1 μg/ml: 88±11%) in comparison with the reference group (serumfree: 11±14%; FCS: 20±19%; p-values<0.001). Incubation in the presence of human C3a and C3a antibodies resulted in a significant decrease (C3a mAb (sc28294) 5 μg/ml: 15±25%; C3a mAb (HM1072) 5 μg/ml: 21±23%), compared to C3a-activated myofibroblasts (p<0.001, p<0.001, respectively). Incubation in the presence of human C5a and C3a antibodies resulted in no significant decrease (C3a mAb (sc28294) 5 μg/ml: 89±14%; C3a mAb (HM1072) 5 μg/ml: 75±22%), compared to C5a-activated myofibroblasts (p=0.167, p=0.855, respectively). Incubation in the presence of human C3a, C5a and C3a antibodies resulted in no significant decrease (C3a mAb (sc28294) 5 μg/ml: 76±22%; C3a mAb (HM1072) 5 μg/ml: 94±13%), compared to C5a and C3a-activated myofibroblasts (p=0.165, p=0.301, respectively). As shown in FIGS. 37 and 38, the C3a antibodies were found to have a positive effect on myofibroblasts activation in the presence of 10% FCS (C3a mAb (sc28294) 5 μg/ml: 61±29%; C3a mAb (HM1072) 5 μg/ml: 27±16%). Compared to 10% FCS incubated human corneal keratocytes, differences were significant for C3a mAb (sc28294) 5 μg/ml (p=0.002, p=0.241, respectively). As shown in FIGS. 37 and 38, the C3a antibodies were found to have a positive effect on myofibroblasts activation, compared to DMEM without FCS incubated human corneal keratocytes (serumfree control), in all concentrations (C3a mAb (sc28294) 5 μg/ml: 42±35%; C3a mAb (HM1072) 5 μg/ml: 24±16%). Compared to serumfree control human corneal keratocytes, differences were significant (p<0.001, p=0.007, respectively). Thus, the C3a antibodies sc28294 (Santa Cruz Biotechnology) and HM1072 (Hycult Biotech) in concentrations of 5 μg/ml were responsible for causing inhibition of myofibroblasts activated by C3a, but not by C5a nor C3a and C5a combined. Bar=Standard error of the mean.

### Example 37

Mouse C5L2 Protein Fragment Reduces the Formation of Corneal Fibrosis after Alkali-Burn of the Cornea in Mice

To explore the potential functional role of mouse C5L2 protein fragment (mC5L2), according to SEQ ID No.: 19, in the treatment of a subject having an ocular wound or fibrosis, the effect of its presence was examined in an in vivo corneal alkali-burn mouse model. C57/BL6 mice (6-8 weeks old) were treated according to a standardized mouse model of corneal alkali-burn under intraperitoneal general anesthesia (Saika S et al. Am J Pathol 2005). A filter paper, measuring 1.5 mm in diameter, soaked with 2 μl M NaOH (sodium hydroxide) was placed, under stereomicroscopic view, on the central cornea of the right mouse eye for 2 minutes to induce a corneal alkali-burn. Immediately after corneal alkali-burn the treated eyes received either phosphate-buffered saline (PBS) and 0.3% ofloxacin ointment (on day 2, 4, 6 and 8) (PBS/control group); or PBS and 0.3% ofloxacin ointment (on day 2, 4, 6 and 8) and 1.5 μg/ml mC5L2 eye drops 5 times a day (during the entire follow-up period) (PBS with mC5L2 treatment group).

The course of wound healing of the ‘PBS/control group’ and ‘PBS with mC5L2 treatment group’ was examined 5, 10 and 20 days after corneal alkali-burn by gene expression. A list of differentially expressed genes, attained from mouse corneas and generated from a gene expression Clariom S mouse microarray, between the ‘PBS/control’ and ‘PBS with mC5L2 treatment’ group, are shown in Table 9 (day 5), Table 10 (day 10) and Table 11 (day 20). Strongest gene expression differences were observed on day 10 after corneal alkali-burn, accordingly the 100 most significant functional annotations to the differentially expressed genes are listed in Table 12. Thus, the mouse C5L2 protein fragment (mC5L2) was responsible for affecting wound healing and fibrogenesis after corneal alkali-burn in mice by influencing the gene expression, amongst others, of extracellular matrix organization, collagen metabolic processes, cellular responses to growth factors, transforming growth factor beta (receptor) signaling and smooth muscle cell differentiation.

The clinical manifestation of the corneal fibrosis, 20 days after corneal alkali-burn, was evaluated by using established corneal fibrosis grading systems according to Cowell (Cowell B A et al. ILAR J 1999), McDonald (McDonald T O et al. Eye irritation 1997, p 579-582: Marzulli F N et al. Dermatotoxicology and pharmacology) and Drew (Drew A F et al., Invest Ophthalmol Vis Sci. 2000).

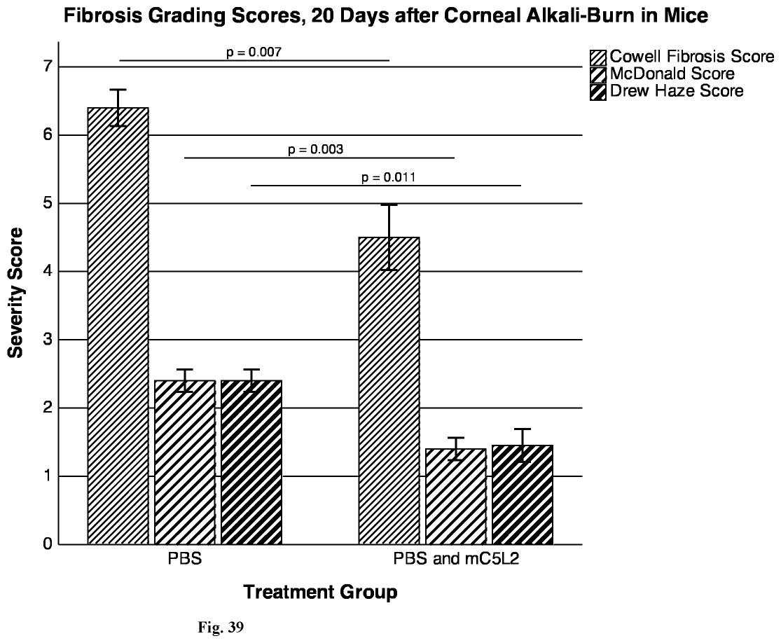

The Cowell score is the sum of grading the area of fibrosis (0: None, 1: 1-25%, 2: 26-50%, 3: 51-75%, 4: 76-100%), the density of opacity (0: Clear, 1: Slight cloudiness, details of pupil and iris discernible, 2: Cloudy, but outline of the iris and pupil remains visible, 3: Cloudy, opacity not uniform, 4: Uniform opacity) and the surface regularity (0: Smooth, 1: Slight surface irregularity, 2: Rough surface, some swelling, 3: Significant swelling, crater or descemetocele formation, 4: Perforation or serious descemetocele). The McDonald-(Shadduck) score is grading of the transparency of the cornea (0: No visible lesion, 1: Some loss of transparency. The underlying structures are clearly visible with diffuse illumination, 2: Moderate loss of transparency. With diffuse illumination the underlying structures are barely visible, but can still be examined and graded, 3: Severe loss of transparency. With diffuse illumination the underlying structures are not visible when viewed through the lesion and evaluation of them is impaired). The Drew haze score is grading of the corneal haze (0: complete clarity, ½ minimal haze, 1: mild haze, 2: significant haze, 3: complete obscuration of the anterior chamber and iris). The grading scores according to Cowell, McDonald and Drew of the corneal fibrosis, 20 days after corneal alkali-burn, of the ‘PBS/control’ and ‘PBS with mC5L2 treatment’ group are shown in FIG. 39. The treatment with the mouse C5L2 protein fragment (mC5L2) resulted in significantly reduced scores (Cowell: 4.5±1.5, McDonald: 1.4±0.5, Drew: 1.5±0.8), compared to PBS-treated controls (Cowell: 6.4±0.8, McDonald: 2.4±0.5, Drew: 2.4±0.5; p=0.007, p=0.003 and p=0.011, respectively).

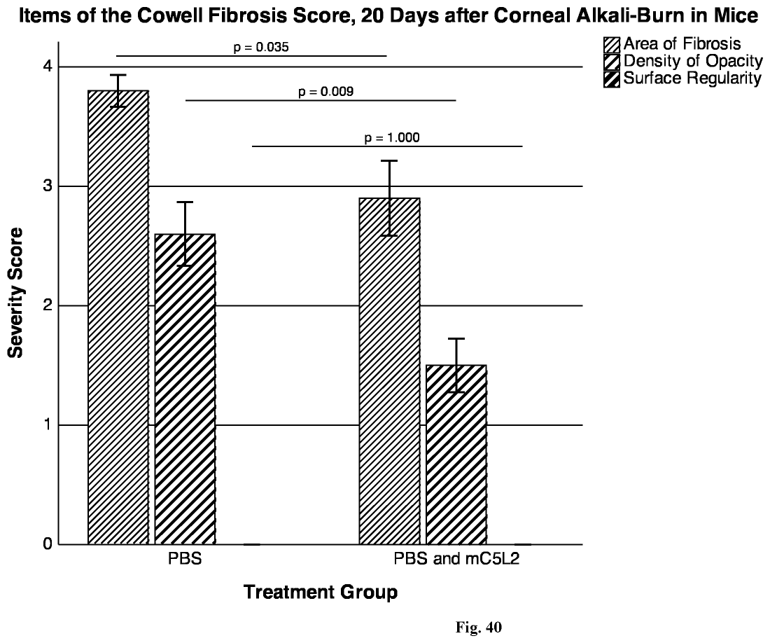

Regarding the items of the Cowell score, as shown in FIG. 40, the treatment with mC5L2 resulted in significantly reduced area and density of opacity (area of fibrosis: 2.9±1.0 vs. 3.8±0.4, p=0.035; density of opacity: 1.5±0.7 vs. 2.6±0.8, p=0.009), but not surface regularity (surface regularity: 0.0±0.0 vs. 0.0±0.0, p=1.000), compared to PBS-treated controls. Thus, the mouse C5L2 protein fragment (mC5L2) was responsible for causing inhibition of the corneal fibrosis after alkali-burn in mice, which resulted in a reduced density of opacity and less haze with greater corneal transparency, and smaller fibrotic areas. Bar=Standard error of the mean.

Wound healing and fibrosis, 20 days after corneal alkali-burn, of the ‘PBS/control’ and ‘PBS with mC5L2 treatment’ group was examined by protein expression. A list of differentially expressed proteins, attained from mouse corneas and generated from a protein expression scioDiscover antibody microarray, between the ‘PBS/control’ and ‘PBS with mC5L2 treatment’ group, are shown in Table 13. The functional annotations to the differentially expressed proteins are listed in Table 14. Thus, the mouse C5L2 protein fragment (mC5L2) was responsible for affecting wound healing and fibrogenesis after corneal alkali-burn in mice by influencing the protein expression, amongst others, of responses to wounding, immune system processes, collagen catabolic processes, as well as extracellular matrix disassembly and organization.

In summary, the mouse C5L2 protein fragment (mC5L2) was responsible for causing inhibition of fibrosis after corneal alkali-burn in mice by intervening diverse biological processes, as listed in Tab. 12 and 14, which resulted in a smaller area and less opacification of the fibrosis on cornea.

TABLE 2
Differentiation of gene expression of human corneal keratocytes after 24 hours of
incubation with human C3a 0.1 μg/ml and serum-free medium (control).

| | C3a | serumfree | | | C3a | serumfree | |
|---|---|---|---|---|---|---|---|
| Gene | Avg | Avg | Fold | Gene | Avg | Avg | Fold |
| Symbol | (log2) | (log2) | Change | Symbol | (log2) | (log2) | Change |
| MYO1E | 4.33 | 6.52 | −4.55 | EPB41L3 | 5.83 | 4.83 | 2 |
| EPHA4 | 3.64 | 5.57 | −3.82 | MED12L | 7.47 | 6.47 | 2 |
| PPM1B | 3.49 | 5.21 | −3.29 | LIMCH1 | 12 | 10.99 | 2 |
| ZNF573 | 5.67 | 7.35 | −3.22 | RP11-93O14.2 | 7.11 | 6.1 | 2.01 |
| JAK2 | 4.95 | 6.46 | −2.85 | SMAD6 | 9.15 | 8.14 | 2.01 |
| RPS3A | 5.5 | 7 | −2.83 | ANKS3 | 8.2 | 7.19 | 2.02 |
| ART1 | 4.66 | 6.09 | −2.71 | SLC27A2 | 6.46 | 5.44 | 2.04 |
| CCBE1 | 4.73 | 6.11 | −2.6 | ANKRD1 | 8.96 | 7.94 | 2.04 |
| TUBGCP3 | 4 | 5.37 | −2.6 | VIPR1 | 7.25 | 6.22 | 2.04 |
| WDR1 | 4.58 | 5.95 | −2.59 | OR4F29 | 6.2 | 5.17 | 2.05 |
| EDA | 4.19 | 5.57 | −2.59 | OR4F16 | 6.2 | 5.17 | 2.05 |
| EXT1; | 8.7 | 10.07 | −2.58 | MX2 | 6.77 | 5.73 | 2.06 |
| hunera | | | | | | | |
| SGMS2 | 4.2 | 5.53 | −2.52 | CDH6 | 9.56 | 8.52 | 2.06 |
| C18orf63 | 3.69 | 5.02 | −2.51 | GLB1L | 7.79 | 6.75 | 2.06 |
| WNK2 | 5.95 | 7.26 | −2.47 | HGD | 5.13 | 4.07 | 2.08 |
| NTM | 9.16 | 10.45 | −2.45 | WRB | 5.35 | 4.29 | 2.09 |
| OR12D2 | 3.13 | 4.42 | −2.45 | TECPR2 | 6.82 | 5.76 | 2.1 |
| CYP2R1 | 3.91 | 5.18 | −2.41 | DACT1 | 5.64 | 4.57 | 2.1 |
| SLC44A5 | 4.72 | 5.98 | −2.39 | NCKAP5 | 6.63 | 5.56 | 2.1 |
| SECTM1 | 4.39 | 5.64 | −2.39 | ZBBX | 3.93 | 2.85 | 2.13 |
| MICAL2 | 4.44 | 5.67 | −2.36 | RGS20 | 6.26 | 5.16 | 2.14 |
| BTLA | 3.78 | 5 | −2.34 | ASCL3 | 4.99 | 3.89 | 2.14 |
| IGIP | 5.71 | 6.93 | −2.33 | ZNF502 | 5.78 | 4.68 | 2.15 |
| SCIN | 4.05 | 5.27 | −2.32 | UBE2D3 | 5.1 | 3.99 | 2.15 |
| POGZ | 6.73 | 7.93 | −2.3 | DIRAS3 | 9.25 | 8.13 | 2.17 |
| MXD1 | 4.8 | 5.99 | −2.28 | LYPD6B | 8.76 | 7.64 | 2.18 |
| TOPAZ1 | 4.11 | 5.3 | −2.28 | MAL2 | 5.7 | 4.57 | 2.19 |
| OR52E8 | 4.22 | 5.4 | −2.28 | INPP5E | 8.16 | 7.03 | 2.19 |
| C18orf65 | 4.19 | 5.37 | −2.27 | SEMA3D | 6.56 | 5.43 | 2.19 |
| AF131215.3 | 4.83 | 6.02 | −2.27 | WNT2 | 7.49 | 6.34 | 2.22 |
| PLGLB2 | 4.29 | 5.46 | −2.26 | TRHDE | 5.17 | 4 | 2.26 |
| ZNF436- | 4.72 | 5.89 | −2.25 | RBKS | 5.85 | 4.67 | 2.26 |
| AS1 | | | | | | | |
| TMEM14EP | 2.9 | 4.06 | −2.23 | TAS2R50 | 4.64 | 3.47 | 2.26 |
| HDLBP | 4.94 | 6.09 | −2.23 | THSD4 | 5.97 | 4.8 | 2.26 |
| OR52E1 | 3.82 | 4.96 | −2.22 | DMD | 8.61 | 7.39 | 2.33 |
| TMEM204 | 4.02 | 5.17 | −2.21 | CHTF8 | 5.57 | 4.34 | 2.34 |
| EFHC2 | 3.14 | 4.28 | −2.21 | KIRREL3 | 5.29 | 4.05 | 2.36 |
| FEZF2 | 4.4 | 5.53 | −2.2 | ADAM28 | 5.08 | 3.84 | 2.36 |
| TAF1 | 4.62 | 5.76 | −2.19 | FAM46C | 5.68 | 4.42 | 2.4 |
| KLHDC4 | 4.91 | 6.04 | −2.19 | TINAG | 5.14 | 3.87 | 2.4 |
| ITGB2 | 3.95 | 5.08 | −2.18 | CDNF | 5.34 | 4.07 | 2.41 |
| ARR3 | 4.39 | 5.51 | −2.17 | CHRM3 | 6.97 | 5.7 | 2.42 |
| C1QTNF6 | 5.09 | 6.2 | −2.17 | RPS6KA5 | 8.17 | 6.86 | 2.47 |
| TTC39B | 4.4 | 5.51 | −2.16 | IGF2 | 7.06 | 5.72 | 2.54 |
| FOXO1 | 3.45 | 4.56 | −2.16 | PTGFRN | 8.86 | 7.5 | 2.56 |
| DOCK10 | 5.49 | 6.58 | −2.13 | SPIB | 5.16 | 3.79 | 2.6 |
| DUX4 | 4.31 | 5.4 | −2.13 | OSBPL1A | 4.56 | 3.18 | 2.61 |
| RAB39B | 3.21 | 4.3 | −2.13 | ANKRD18B | 6.05 | 4.65 | 2.63 |
| BIRC3 | 4.03 | 5.12 | −2.12 | FLOT2 | 6.49 | 5.08 | 2.66 |
| SF1 | 7.68 | 8.76 | −2.12 | ZNF546 | 5.66 | 4.24 | 2.68 |
| KIRREL3 | 6.66 | 7.74 | −2.11 | NME5 | 5.58 | 4.14 | 2.72 |
| COL6A2 | 4.5 | 5.57 | −2.1 | PDE1C | 11.56 | 10.1 | 2.74 |
| TFRC | 3.54 | 4.6 | −2.09 | SERPINB2 | 8.32 | 6.85 | 2.78 |
| PPEF2 | 4.53 | 5.58 | −2.08 | RFX4 | 5.77 | 4.29 | 2.81 |
| ST8SIA1 | 3.86 | 4.92 | −2.08 | SEMA5A | 7.66 | 6.11 | 2.92 |
| UTS2B | 4.78 | 5.83 | −2.06 | RGS7BP | 6.83 | 5.27 | 2.94 |
| HSFX1 | 5.77 | 6.81 | −2.06 | IL6 | 9.52 | 7.86 | 3.15 |
| LYZL6 | 3.42 | 4.47 | −2.06 | RARB | 6.21 | 4.52 | 3.23 |
| MBOAT2 | 4.97 | 6.01 | −2.06 | ANKRD44 | 5.66 | 3.93 | 3.32 |
| CHAC1 | 10.11 | 11.15 | −2.05 | SULF1 | 11.88 | 9.32 | 5.89 |
| TPD52 | 8.55 | 9.58 | −2.04 | | | | |
| XRCC5 | 6.8 | 7.83 | −2.04 | | | | |
| RBMS1 | 4.69 | 5.72 | −2.04 | | | | |
| IFNA7 | 3.2 | 4.22 | −2.03 | | | | |
| SCAPER | 3.66 | 4.68 | −2.03 | | | | |
| LINGO4 | 4.32 | 5.34 | −2.03 | | | | |
| ANKRD36 | 6.9 | 7.92 | −2.03 | | | | |
| C5orf66 | 5.1 | 6.12 | −2.03 | | | | |
| SQSTM1 | 3.88 | 4.9 | −2.02 | | | | |
| LTBP4 | 8.25 | 9.26 | −2.02 | | | | |
| NEK5 | 4.09 | 5.11 | −2.02 | | | | |
| MYO1D | 5.96 | 6.98 | −2.02 | | | | |
| ANK2 | 5.05 | 6.06 | −2.02 | | | | |
| HCRP1 | 3.59 | 4.6 | −2.01 | | | | |
| SLC38A9 | 4.82 | 5.83 | −2.01 | | | | |
| SUMO4 | 7.16 | 8.17 | −2.01 | | | | |
| KSR2 | 4.19 | 5.2 | −2.01 | | | | |
| PILRB | 4.1 | 5.11 | −2.01 | | | | |
| STK32C | 7.67 | 8.67 | −2 | | | | |
| SPIRE2 | 5.63 | 6.63 | −2 | | | | |
| DCST1 | 5.33 | 6.34 | −2 | | | | |
| UNC13A | 3.8 | 4.8 | −2 | | | | |

TABLE 3
Differentiation of gene expression of human corneal keratocytes after 24 hours of
incubation with human C5a 0.1 μg/ml and serum-free medium (control).

| | C5a | serumfree | | | C5a | serumfree | |
|---|---|---|---|---|---|---|---|
| Gene | Avg | Avg | Fold | Gene | Avg | Avg | Fold |
| Symbol | (log2) | (log2) | Change | Symbol | dog2) | dog2) | Change |
| CAMKMT | 3.55 | 5.74 | −4.56 | CLEC4M | 6.79 | 5.78 | 2.01 |
| RAB28 | 4.55 | 6.29 | −3.34 | ZNF582-AS1 | 5.45 | 4.43 | 2.02 |
| JAK2 | 4.88 | 6.46 | −3 | TMEM212 | 4.34 | 3.32 | 2.03 |
| PKP4 | 5.6 | 7.17 | −2.96 | TDRD12 | 4.17 | 3.15 | 2.03 |
| CYP2R1 | 3.66 | 5.18 | −2.87 | CDON | 8.32 | 7.3 | 2.04 |
| BACH2 | 2.98 | 4.45 | −2.77 | MTNRIA | 5.48 | 4.45 | 2.04 |
| PLEKHA6 | 4.57 | 6.03 | −2.75 | SORCS1 | 5.97 | 4.94 | 2.04 |
| EXT1;hunera | 8.62 | 10.07 | −2.72 | RPS6KA5 | 7.9 | 6.86 | 2.04 |
| LCORL | 4.2 | 5.61 | −2.65 | TSGA10IP | 7.6 | 6.57 | 2.04 |
| PSMB8-AS1 | 5.38 | 6.78 | −2.64 | CSPG5 | 5.61 | 4.57 | 2.06 |
| CSMD1 | 3.59 | 4.97 | −2.6 | SLC22A18AS | 7.81 | 6.76 | 2.06 |
| RPS3A | 5.63 | 7 | −2.58 | OR9K2 | 8.03 | 6.96 | 2.09 |
| OR5F1 | 4.32 | 5.66 | −2.55 | ERV3-1 | 8 | 6.93 | 2.1 |
| MTFP1 | 4.72 | 6.04 | −2.5 | PCP2 | 7.41 | 6.33 | 2.12 |
| CD53 | 3.61 | 4.91 | −2.47 | PTPRR | 5.11 | 4.01 | 2.14 |
| LSM6 | 5.63 | 6.91 | −2.43 | GAGE2D | 4.81 | 3.72 | 2.14 |
| TBC1D3 | 8.02 | 9.29 | −2.4 | IFNA8 | 6.22 | 5.12 | 2.14 |
| TAAR2 | 3.92 | 5.18 | −2.4 | TMEM179 | 5.48 | 4.37 | 2.15 |
| POGZ | 5.47 | 6.73 | −2.39 | PCSK2 | 4.47 | 3.36 | 2.15 |
| RAD54L | 5.37 | 6.62 | −2.38 | WDR78 | 6.28 | 5.16 | 2.17 |
| MARCH1 | 3.46 | 4.71 | −2.37 | FLOT2 | 6.22 | 5.08 | 2.2 |
| ITGB6 | 4 | 5.24 | −2.36 | GOLGA8N | 6.57 | 5.41 | 2.22 |
| TBC1D3H | 8.9 | 10.14 | −2.35 | IFIH1 | 8.31 | 7.15 | 2.23 |
| DACH1 | 3.3 | 4.52 | −2.33 | MPZL3 | 8.2 | 7.04 | 2.23 |
| PCDHB1 | 3.76 | 4.98 | −2.33 | THEM5 | 6.47 | 5.3 | 2.25 |
| NR2C2 | 5.61 | 6.81 | −2.3 | FNDC7 | 4.95 | 3.78 | 2.25 |
| SF1 | 7.56 | 8.76 | −2.3 | AACSP1 | 5.81 | 4.63 | 2.27 |
| TRIM10 | 5.42 | 6.6 | −2.26 | METTL7B | 7.45 | 6.26 | 2.29 |
| EPHA4 | 4.56 | 5.73 | −2.25 | SELL | 4.79 | 3.6 | 2.29 |
| TTC39B | 4.35 | 5.51 | −2.24 | RAB11A | 4.57 | 3.36 | 2.31 |
| LGI2 | 4.2 | 5.36 | −2.24 | INTS1 | 5.7 | 4.48 | 2.33 |
| TPK1 | 4.62 | 5.78 | −2.24 | RBKS | 5.91 | 4.67 | 2.36 |
| NPL | 3.48 | 4.64 | −2.23 | PLCB1 | 5.66 | 4.42 | 2.36 |
| PNPLA7 | 3.19 | 4.34 | −2.21 | ENKD1 | 7.33 | 6.09 | 2.36 |
| CCDC84 | 8.31 | 9.44 | −2.2 | OR8H2 | 5.07 | 3.82 | 2.37 |
| TAS2R19 | 4.08 | 5.22 | −2.19 | KCNN3 | 5.56 | 4.24 | 2.51 |
| C16orf72 | 5.28 | 6.41 | −2.19 | TINAG | 5.23 | 3.87 | 2.55 |
| HDLBP | 4.97 | 6.09 | −2.17 | DLK1 | 5.27 | 3.91 | 2.56 |
| CLASP2 | 4.46 | 5.57 | −2.17 | PDK4 | 4.72 | 3.25 | 2.77 |
| ANXA2R | 6.86 | 7.97 | −2.17 | CCDC173 | 5.72 | 4.24 | 2.79 |
| SLC44A5 | 4.86 | 5.98 | −2.17 | HBB | 5.99 | 4.47 | 2.87 |
| XRCC5 | 7.57 | 8.68 | −2.16 | LAP3 | 5.87 | 3.75 | 4.36 |
| HESX1 | 4.6 | 5.7 | −2.15 | | | | |
| EXT1; spaw1a | 6.42 | 7.53 | −2.15 | | | | |
| AKR1C8P | 3.84 | 4.94 | −2.15 | | | | |
| ATP13A3 | 5.33 | 6.43 | −2.15 | | | | |
| OXCT2P1 | 5.72 | 6.82 | −2.14 | | | | |
| ZBTB9 | 8.17 | 9.27 | −2.14 | | | | |
| TMEM236 | 3.75 | 4.83 | −2.12 | | | | |
| CYP39A1 | 3.35 | 4.43 | −2.12 | | | | |
| SLC17A1 | 3.95 | 5.03 | −2.11 | | | | |
| STK33 | 3.45 | 4.52 | −2.1 | | | | |
| GALNTL5 | 3.87 | 4.94 | −2.1 | | | | |
| TAS2R31 | 7.49 | 8.56 | −2.09 | | | | |
| CPLX4 | 4.95 | 6.01 | −2.09 | | | | |
| CC2D2A | 3.31 | 4.37 | −2.09 | | | | |
| MXD1 | 4.93 | 5.99 | −2.08 | | | | |
| MS4A4E | 4.65 | 5.7 | −2.07 | | | | |
| TNKS | 5.98 | 7.03 | −2.06 | | | | |
| KRT23 | 4.56 | 5.6 | | | | | |
| OR12D2 | 3.39 | 4.42 | −2.04 | | | | |
| HSFX2 | 6.64 | 7.67 | −2.04 | | | | |
| TJP1 | 4.59 | 5.61 | −2.04 | | | | |
| PLGLB2 | 4.44 | 5.46 | −2.04 | | | | |
| LRRC20 | 5.23 | 6.25 | −2.03 | | | | |
| KIRREL3 | 6.72 | 7.74 | −2.02 | | | | |
| PAQR6 | 4.86 | 5.87 | −2.02 | | | | |
| KDM4C | 5.62 | 6.63 | −2.02 | | | | |
| PTAFR | 4.49 | 5.5 | −2.02 | | | | |
| NEK5 | 4.09 | 5.11 | −2.02 | | | | |
| ZNF721 | 7.83 | 8.84 | −2.02 | | | | |
| PYY | 5.03 | 6.04 | −2.02 | | | | |
| TBC1D3G | 7.84 | 8.85 | −2.01 | | | | |
| TCERG1 | 4.02 | 5.03 | −2.01 | | | | |
| ZNF436- | 4.88 | 5.89 | −2 | | | | |
| AS1 | | | | | | | |
| ERAS | 5.21 | 6.22 | −2 | | | | |
| NAIP | 6.31 | 7.31 | −2 | | | | |
| OR5T1 | 4.38 | 5.38 | −2 | | | | |
| SLC24A2 | 4.49 | 3.48 | 2 | | | | |

TABLE 4
Differentiation of gene expression of human corneal keratocytes after 24 hours of
incubation with human C3a/C5a 0.1 μg/ml and serum-free medium.

| | C3a/C5a | serumfree | | | C3a/C5a | serumfree | |
|---|---|---|---|---|---|---|---|
| Gene | Avg | Avg | Fold | Gene | Avg | Avg | Fold |
| Symbol | (log2) | (log2) | Change | Symbol | (log2) | (log2) | Change |
| OCRL | 4.57 | 6.47 | −3.71 | DZANK1 | 5.76 | 4.75 | 2.01 |
| TJP1 | 3.98 | 5.61 | −3.1 | ADAM18 | 5.11 | 4.11 | 2.01 |
| MYO1E | 4.9 | 6.52 | −3.07 | RGS7BP | 6.29 | 5.27 | 2.02 |
| TAF1 | 4.2 | 5.76 | −2.93 | KCNAB2 | 9.53 | 8.51 | 2.02 |
| HYOU1 | 3.64 | 5.19 | −2.93 | TCAP | 9.99 | 8.97 | 2.02 |
| SYT1 | 5.24 | 6.79 | −2.93 | PVALB | 5.61 | 4.59 | 2.03 |
| SCAPER | 3.23 | 4.68 | −2.72 | HOXB5 | 6.84 | 5.82 | 2.03 |
| HK2 | 7.68 | 9.12 | −2.72 | SPATA45 | 5.33 | 4.31 | 2.03 |
| NTM | 9.05 | 10.45 | −2.63 | MS4A5 | 5.87 | 4.85 | 2.03 |
| CYP2R1 | 3.8 | 5.18 | −2.61 | SYT12 | 5.42 | 4.4 | 2.03 |
| CST9L | 3.64 | 5.01 | −2.58 | TACSTD2 | 5.08 | 4.06 | 2.03 |
| LTBP2 | 6.22 | 7.58 | −2.56 | NME5 | 5.16 | 4.14 | 2.04 |
| OR10AG1 | 6.05 | 7.38 | −2.51 | IL1B | 10.45 | 9.42 | 2.04 |
| MAGEE1 | 4.9 | 6.22 | −2.5 | OR2B2 | 4.36 | 3.34 | 2.04 |
| EPHA4 | 4.27 | 5.57 | −2.48 | CDH6 | 9.55 | 8.52 | 2.04 |
| PLEKHA6 | 4.76 | 6.03 | −2.42 | ATP10A | 5.43 | 4.4 | 2.05 |
| MXD1 | 4.72 | 5.99 | −2.4 | EPB41 | 6.82 | 5.79 | 2.05 |
| POGZ | 6.67 | 7.93 | −2.4 | ATP4B | 5.04 | 4.01 | 2.05 |
| LAPTM4A | 4.42 | 5.67 | −2.39 | KCNN3 | 5.27 | 4.24 | 2.05 |
| ARPP21 | 3.98 | 5.23 | −2.38 | CXCL8 | 7.26 | 6.22 | 2.06 |
| STC1 | 10.62 | 11.86 | −2.37 | PATEl | 5.78 | 4.74 | 2.06 |
| CADPS | 3.16 | 4.4 | −2.36 | FLG | 4.62 | 3.57 | 2.06 |
| MTFP1 | 4.8 | 6.04 | −2.35 | NTF3 | 9.78 | 8.74 | 2.06 |
| YEATS2 | 3.24 | 4.47 | −2.35 | ST6GAL1 | 7.55 | 6.49 | 2.08 |
| ZNF512 | 4.99 | 6.21 | −2.33 | TES | 11.25 | 10.19 | 2.08 |
| EDA | 4.37 | 5.57 | −2.3 | LAP3 | 4.81 | 3.75 | 2.08 |
| RAB2A | 4.53 | 5.73 | −2.29 | LINC01588 | 5.54 | 4.48 | 2.09 |
| OR5F1 | 4.47 | 5.66 | −2.29 | CNTNAP3P2 | 7.46 | 6.39 | 2.09 |
| PTPRB | 6.28 | 7.48 | −2.29 | CHTF8 | 5.41 | 4.34 | 2.09 |
| BNIP3 | 13.79 | 14.97 | −2.27 | RASAL3 | 8.24 | 7.17 | 2.1 |
| KIRREL3 | 6.55 | 7.74 | −2.27 | GPRC5B | 11.18 | 10.1 | 2.12 |
| CEP97 | 6.16 | 7.34 | −2.27 | ANO9 | 5.04 | 3.96 | 2.12 |
| GMDS | 6.87 | 8.04 | −2.26 | SPRR3 | 5.24 | 4.15 | 2.13 |
| ZNF165 | 3.98 | 5.15 | −2.25 | TTC21A | 6.56 | 5.47 | 2.13 |
| VCAN | 9.69 | 10.86 | −2.25 | F2RL1 | 7.64 | 6.54 | 2.14 |
| TPD52 | 8.42 | 9.58 | −2.23 | RBM26 | 6.59 | 5.49 | 2.14 |
| TEF | 5.97 | 7.12 | −2.23 | C7orf69 | 7.73 | 6.63 | 2.14 |
| GPR173 | 3.83 | 4.98 | −2.23 | ZNF527 | 6.05 | 4.95 | 2.14 |
| FAM151B | 4.88 | 6.02 | −2.2 | ICA1 | 4.71 | 3.61 | 2.14 |
| PCDHB13 | 5.37 | 6.51 | −2.2 | ITGB4 | 6.02 | 4.91 | 2.15 |
| ZBTB9 | 8.14 | 9.27 | −2.2 | SNX29P2 | 8.01 | 6.9 | 2.15 |
| TMEM204 | 4.03 | 5.17 | −2.2 | ESAM | 5.93 | 4.83 | 2.15 |
| THSD4 | 8.41 | 9.55 | −2.2 | MRAS | 6.42 | 5.31 | 2.16 |
| MYO1D | 5.85 | 6.98 | −2.19 | EPB41L3 | 5.94 | 4.83 | 2.16 |
| SERTAD3 | 7.52 | 8.65 | −2.18 | EPHA7 | 6.18 | 5.07 | 2.16 |
| PCDHB1 | 3.86 | 4.98 | −2.18 | CDRT1 | 5.73 | 4.61 | 2.17 |
| SGMS2 | 4.41 | 5.53 | −2.17 | HLA-DQB2 | 7.85 | 6.73 | 2.17 |
| DNM3 | 3.83 | 4.95 | −2.17 | DDX11 | 6.55 | 5.43 | 2.18 |
| WNK2 | 6.15 | 7.26 | −2.16 | ASCL3 | 5.01 | 3.89 | 2.18 |
| AGR2 | 3.73 | 4.84 | −2.15 | RARRES1 | 9.61 | 8.48 | 2.18 |
| NLRP3 | 3.61 | 4.72 | −2.15 | SSPN | 6.53 | 5.4 | 2.19 |
| ITGB6 | 4.13 | 5.24 | −2.15 | LYPD6 | 7.01 | 5.88 | 2.19 |
| LINC01537 | 3.86 | 4.96 | −2.15 | SERPINA4 | 4.88 | 3.75 | 2.19 |
| SKP2 | 5.3 | 6.41 | −2.15 | PHOSPHO1 | 5.76 | 4.62 | 2.19 |
| EPHA4 | 4.63 | 5.73 | −2.15 | C1orf198 | 10.42 | 9.28 | 2.2 |
| RASSF9 | 3.26 | 4.37 | −2.15 | DACT1 | 5.72 | 4.57 | 2.21 |
| NAIP | 5.78 | 6.88 | −2.14 | SPNS3 | 8.1 | 6.96 | 2.21 |
| NLRP9 | 4.37 | 5.46 | −2.13 | AIM1L | 6.35 | 5.2 | 2.22 |
| CSTA | 5.21 | 6.29 | −2.13 | TAS2R50 | 4.62 | 3.47 | 2.23 |
| DGKD | 5.05 | 6.13 | −2.11 | OR4F6 | 4.36 | 3.21 | 2.23 |
| PIP4K2A | 4.4 | 5.48 | −2.11 | LAIR1 | 5.33 | 4.17 | 2.23 |
| OR5T1 | 4.31 | 5.38 | −2.11 | IL7 | 5.07 | 3.91 | 2.24 |
| KLF17 | 3.78 | 4.85 | −2.11 | KCNK17 | 7.92 | 6.76 | 2.24 |
| RPS3A | 5.92 | 7 | −2.1 | RAB7B | 7.87 | 6.7 | 2.24 |
| TTC25 | 5.47 | 6.54 | −2.09 | ANKRD18B | 5.83 | 4.65 | 2.25 |
| CSMD1 | 3.91 | 4.97 | −2.08 | C10orf10 | 7.75 | 6.57 | 2.26 |
| DNASE1 | 5.08 | 6.14 | −2.08 | BLK | 7.84 | 6.65 | 2.27 |
| CCDC159 | 4.85 | 5.9 | −2.08 | WNT2 | 7.53 | 6.34 | 2.27 |
| DDIT4 | 11 | 12.06 | −2.08 | ITGB8 | 6.64 | 5.45 | 2.28 |
| QRICH1 | 6.44 | 7.49 | −2.08 | SORCS1 | 6.13 | 4.94 | 2.28 |
| HDLBP | 5.04 | 6.09 | −2.08 | THEM5 | 6.51 | 5.3 | 2.3 |
| MYRIP | 7.02 | 8.08 | −2.07 | DEFB105B | 5.28 | 4.08 | 2.3 |
| NLRP1 | 8 | 9.05 | −2.07 | EVPLL | 5.74 | 4.53 | 2.3 |
| BIRC3 | 4.07 | 5.12 | −2.06 | LILRB3 | 4.9 | 3.69 | 2.31 |
| BACH2 | 3.41 | 4.45 | −2.06 | ZSCAN31 | 7.19 | 5.97 | 2.32 |
| PEX5L | 4.88 | 5.92 | −2.06 | OR52E6 | 5.52 | 4.31 | 2.33 |
| KDM4C | 5.59 | 6.63 | −2.05 | PDE1C | 11.34 | 10.1 | 2.36 |
| PITPNM2 | 5.51 | 6.55 | −2.05 | UGT2B10 | 4.4 | 3.14 | 2.39 |
| ARHGAP15 | 3.44 | 4.47 | −2.04 | RGS20 | 6.43 | 5.16 | 2.41 |
| VLDLR | 8.63 | 9.66 | −2.04 | HTR5A | 5.16 | 3.89 | 2.42 |
| AP1S1 | 5.56 | 6.59 | −2.04 | PABPC4L | 7.6 | 6.32 | 2.42 |
| EFCAB13 | 5.43 | 6.45 | −2.04 | CSPG5 | 5.85 | 4.57 | 2.43 |
| PRKAA2 | 5.92 | 6.95 | −2.03 | CYTH4 | 6.54 | 5.26 | 2.44 |
| ZNF385D | 5.68 | 6.7 | −2.02 | TMEM212 | 4.61 | 3.32 | 2.45 |
| SRP72 | 4.69 | 5.71 | −2.02 | NCKAP5 | 6.87 | 5.56 | 2.48 |
| | | | | LMOD3 | 4.87 | 3.56 | 2.48 |
| | | | | MS4A12 | 5.93 | 4.61 | 2.5 |
| | | | | Sep 14 | 5.25 | 3.91 | 2.53 |
| | | | | UBE2D3 | 5.34 | 3.99 | 2.55 |
| | | | | SEMA5A | 7.47 | 6.11 | 2.56 |
| | | | | NGB | 5.39 | 4.02 | 2.59 |
| | | | | PDK4 | 4.65 | 3.25 | 2.65 |
| | | | | PTGFRN | 8.91 | 7.5 | 2.65 |
| | | | | TAL1 | 5.78 | 4.37 | 2.65 |
| | | | | ADAM28 | 5.25 | 3.84 | 2.66 |
| | | | | IGF2 | 7.14 | 5.72 | 2.67 |
| | | | | RARB | 5.96 | 4.52 | 2.72 |
| | | | | SERPINB2 | 8.33 | 6.85 | 2.78 |
| | | | | PADI4 | 5.84 | 4.35 | 2.81 |
| | | | | C8orf46 | 6.35 | 4.8 | 2.91 |
| | | | | FLOT2 | 6.63 | 5.08 | 2.92 |
| | | | | IL6 | 9.96 | 7.86 | 4.28 |
| | | | | SULF1 | 12.24 | 9.32 | 7.57 |

TABLE 5
Differentiation of gene expression of human corneal keratocytes after 24 hours of
incubation with human C3a 0.1 μg/ml and human C3a 0.1 μg/ml with human C5L2 0.3 μg/ml.

| | C3a | C3a/C5L2 | | | C3a | C3a/C5L2 | |
|---|---|---|---|---|---|---|---|
| Gene | Avg | Avg | Fold | Gene | Avg | Avg | Fold |
| Symbol | (log2) | (log2) | Change | Symbol | (log2) | (log2) | Change |
| SLC38A9 | 4.82 | 6.65 | −3.56 | NME5 | 5.58 | 4.58 | 2 |
| ART1 | 4.66 | 6.4 | −3.35 | SNRPD2P2 | 5.36 | 4.36 | 2.01 |
| RFX3 | 5.18 | 6.84 | −3.15 | FAM46C | 5.68 | 4.67 | 2.02 |
| TUBGCP3 | 4 | 5.62 | −3.09 | GPR157 | 8.27 | 7.25 | 2.02 |
| WDR1 | 4.58 | 6.15 | −2.97 | SAR1B | 5.25 | 4.23 | 2.03 |
| CLCNKB | 3.79 | 5.36 | −2.97 | AKR1C8P | 4.97 | 3.95 | 2.03 |
| BIRC3 | 4.03 | 5.59 | −2.94 | ZNF812P | 5.46 | 4.43 | 2.04 |
| POLE | 4.69 | 6.23 | −2.9 | KCNAB1 | 5.62 | 4.59 | 2.04 |
| HIPK1 | 5.5 | 7.02 | −2.87 | RFX4 | 5.77 | 4.73 | 2.06 |
| IGIP | 5.71 | 7.23 | −2.86 | TRAF1 | 6.2 | 5.15 | 2.07 |
| UBR3 | 3.41 | 4.91 | −2.83 | BCL7A | 5.01 | 3.95 | 2.09 |
| JAK2 | 4.95 | 6.38 | −2.69 | ZFPM2 | 5.55 | 4.48 | 2.1 |
| RGS16 | 4.34 | 5.7 | −2.57 | HRH1 | 7.06 | 5.98 | 2.1 |
| CYLC2 | 4.53 | 5.86 | −2.52 | TAS2R50 | 4.64 | 3.57 | 2.1 |
| SIDT1 | 4.78 | 6.1 | −2.49 | OTUD6B | 5.5 | 4.43 | 2.11 |
| SLC23A3 | 5.08 | 6.4 | −2.49 | ANKRD44 | 5.66 | 4.58 | 2.11 |
| STON2 | 4.08 | 5.4 | −2.49 | MYH9 | 5.9 | 4.81 | 2.13 |
| MPO | 3.65 | 4.96 | −2.48 | KPNA7 | 5.05 | 3.96 | 2.13 |
| ZNF436-AS1 | 4.72 | 6.02 | −2.48 | SPN | 5.66 | 4.57 | 2.14 |
| CAPN3 | 4.2 | 5.49 | −2.43 | TFAP2C | 5.57 | 4.47 | 2.15 |
| TK2 | 4.61 | 5.87 | −2.39 | CTAGE5 | 7.56 | 6.46 | 2.15 |
| FMN1 | 6.07 | 7.32 | −2.38 | SMIM14 | 9.25 | 8.14 | 2.16 |
| OR13G1 | 4.18 | 5.43 | −2.38 | PLEKHA6 | 5.12 | 4 | 2.18 |
| RFC3 | 5.48 | 6.73 | −2.37 | TCEANC | 4.76 | 3.62 | 2.2 |
| S100A14 | 5.03 | 6.27 | −2.37 | ITGB6 | 4.82 | 3.68 | 2.2 |
| PTPN13 | 5 | 6.21 | −2.31 | MS4A7 | 5.04 | 3.89 | 2.21 |
| ERAP1 | 3.59 | 4.8 | −2.3 | PPP4R4 | 5.03 | 3.88 | 2.22 |
| XKR9 | 4.64 | 5.81 | −2.25 | SIGLEC15 | 5.78 | 4.6 | 2.27 |
| FCHO1 | 3.99 | 5.15 | −2.23 | ZNF846 | 7.41 | 6.22 | 2.28 |
| B3GALT4 | 4.67 | 5.82 | −2.23 | SPOCD1 | 9.99 | 8.73 | 2.39 |
| LRRIQ3 | 3.96 | 5.12 | −2.22 | CCDC79 | 5.29 | 4 | 2.43 |
| PRND | 4.46 | 5.61 | −2.22 | A2BP1 | 4.64 | 3.24 | 2.64 |
| TNFRSF25 | 7.59 | 8.73 | −2.21 | OR4F29 | 6.2 | 4.67 | 2.89 |
| RDH5 | 5.82 | 6.95 | −2.19 | OR4F16 | 6.2 | 4.67 | 2.89 |
| MYRFL | 4.53 | 5.65 | −2.17 | TRHDE | 5.17 | 3.6 | 2.99 |
| KCNK1 | 4.15 | 5.27 | −2.17 | | | | |
| LINC01559 | 4.64 | 5.76 | −2.17 | | | | |
| EPB42 | 4.1 | 5.22 | −2.17 | | | | |
| KLRB1 | 3.91 | 5.02 | −2.16 | | | | |
| CNTNAP3B | 6.16 | 7.26 | −2.15 | | | | |
| HGSNAT | 4.97 | 6.06 | −2.13 | | | | |
| PCSK1 | 4.14 | 5.22 | −2.12 | | | | |
| RIMS2 | 3.53 | 4.62 | −2.12 | | | | |
| PTRH1 | 6.1 | 7.18 | −2.11 | | | | |
| STK32C | 7.67 | 8.75 | −2.11 | | | | |
| EXT1; | 8.7 | 9.77 | −2.11 | | | | |
| hunera | | | | | | | |
| GAS2 | 3.2 | 4.27 | −2.1 | | | | |
| SMAD7 | 6.04 | 7.11 | −2.09 | | | | |
| GPR84 | 3.16 | 4.22 | −2.09 | | | | |
| OR2T3 | 6.18 | 7.24 | −2.08 | | | | |
| ASB17 | 3.93 | 4.98 | −2.08 | | | | |
| EFHC2 | 3.14 | 4.2 | −2.08 | | | | |
| DDC | 4.56 | 5.61 | −2.08 | | | | |
| ALPK3 | 4.02 | 5.07 | −2.07 | | | | |
| BSX | 4.33 | 5.38 | −2.07 | | | | |
| PPM1B | 3.49 | 4.53 | −2.06 | | | | |
| DOCK2 | 4.74 | 5.78 | −2.06 | | | | |
| PTK2 | 5.38 | 6.42 | −2.05 | | | | |
| ANKRD18A | 4.24 | 5.26 | −2.04 | | | | |
| CCBE1 | 4.73 | 5.76 | −2.04 | | | | |
| SPATA32 | 4.63 | 5.66 | −2.03 | | | | |
| TOPAZ1 | 4.11 | 5.13 | −2.03 | | | | |
| CALCR | 3.81 | 4.83 | −2.03 | | | | |
| RSPO2 | 4.12 | 5.14 | −2.03 | | | | |
| PCSK2 | 3.34 | 4.36 | −2.03 | | | | |
| TAS1R2 | 4.43 | 5.44 | −2.03 | | | | |
| ZNF396 | 3.74 | 4.75 | −2.02 | | | | |
| ZNF573 | 5.67 | 6.68 | −2.02 | | | | |
| RGSL1 | 3.35 | 4.37 | −2.02 | | | | |
| PPP2R2B | 4.89 | 5.89 | −2.01 | | | | |
| FABP6 | 4 | 5 | −2 | | | | |
| STPG2 | 3.35 | 4.35 | −2 | | | | |
| SLC15A1 | 3.99 | 4.99 | −2 | | | | |

TABLE 6
Differentiation of gene expression of human corneal keratocytes after 24 hours of
incubation with human C5a 0.1 μg/ml and human C5a 0.1 μg/ml with human C5L2 0.3 μg/ml.

| | C5a | C5a/C5L2 | | | C5a | C5a/C5L2 | |
|---|---|---|---|---|---|---|---|
| Gene | Avg | Avg | Fold | Gene | Avg | Avg | Fold |
| Symbol | (log2) | (log2) | Change | Symbol | (log2) | (log2) | Change |
| HNRNPK | 5.53 | 7.32 | −3.46 | ABCA8 | 7.02 | 6.02 | 2 |
| ANKRD52 | 5.51 | 7.08 | −2.97 | GLRA2 | 4.34 | 3.34 | 2.01 |
| COBLL1 | 4.35 | 5.91 | −2.96 | PRND | 5.41 | 4.39 | 2.02 |
| CD53 | 3.61 | 5.14 | −2.89 | IBA57 | 7.02 | 6 | 2.02 |
| KIRREL3 | 3.96 | 5.45 | −2.81 | CABP1 | 6.01 | 4.98 | 2.04 |
| RAD54L | 5.37 | 6.86 | −2.81 | VPS37B | 7.9 | 6.87 | 2.04 |
| TCF4 | 4.06 | 5.54 | −2.79 | MLXIP | 6.7 | 5.67 | 2.04 |
| NDUFV3 | 3.97 | 5.43 | −2.75 | OR6C74 | 4.31 | 3.28 | 2.05 |
| HNRNPH1 | 6.58 | 8 | −2.67 | PRSS48 | 4.47 | 3.43 | 2.05 |
| RSPO2 | 3.84 | 5.25 | −2.66 | TMIGD2 | 6.55 | 5.51 | 2.06 |
| SNAPC4 | 5.53 | 6.93 | −2.64 | CLEC4G | 7.33 | 6.29 | 2.06 |
| KRTAP4-3 | 3.39 | 4.75 | −2.57 | SELL | 4.79 | 3.74 | 2.07 |
| NPL | 3.48 | 4.83 | −2.54 | PLEKHG6 | 4.34 | 3.29 | 2.07 |
| KRTAP1-5 | 6 | 7.34 | −2.53 | TBATA | 5.97 | 4.91 | 2.08 |
| MARCH1 | 3.46 | 4.77 | −2.47 | FAM209B | 4.98 | 3.93 | 2.08 |
| GPR32 | 4.5 | 5.78 | −2.44 | HBB | 5.99 | 4.94 | 2.08 |
| KRTAP22-2 | 3.95 | 5.23 | −2.42 | FDXR | 5.17 | 4.11 | 2.08 |
| PKP4 | 5.6 | 6.87 | −2.41 | MYOM2 | 5.59 | 4.52 | 2.09 |
| POGZ | 5.38 | 6.65 | −2.41 | CAPN12 | 5.5 | 4.44 | 2.09 |
| JAK2 | 4.88 | 6.13 | −2.38 | FNDC7 | 4.95 | 3.89 | 2.09 |
| TBC1D3H | 8.9 | 10.16 | −2.38 | PREX1 | 8.4 | 7.33 | 2.1 |
| C12orf80 | 3.89 | 5.12 | −2.35 | KCNN3 | 5.56 | 4.49 | 2.11 |
| FABP9 | 4.3 | 5.53 | −2.34 | MYO1G | 4.1 | 3.03 | 2.11 |
| FAM45A | 6.3 | 7.52 | −2.33 | GTF2A1L | 5.24 | 4.17 | 2.11 |
| CNNM3 | 6.35 | 7.57 | −2.32 | FAM197Y1 | 5.08 | 3.99 | 2.12 |
| ZNF446 | 5.83 | 7.04 | −2.31 | HMHB1 | 8.03 | 6.94 | 2.13 |
| SGIP1 | 4.26 | 5.45 | −2.28 | ANKUB1 | 5.36 | 4.26 | 2.14 |
| LRRCC1 | 4.54 | 5.72 | −2.25 | DEFB105B/A | 4.9 | 3.79 | 2.15 |
| CAMKMT | 3.55 | 4.72 | −2.25 | OR11H2 | 4.55 | 3.44 | 2.15 |
| PLA2G2E | 5.67 | 6.83 | −2.23 | PTPRC | 5.89 | 4.79 | 2.15 |
| EFCAB5 | 4.75 | 5.91 | −2.23 | EXD3 | 8.66 | 7.55 | 2.17 |
| AKR1C8P | 3.84 | 4.99 | −2.22 | RLN1 | 4.51 | 3.39 | 2.17 |
| CUL9 | 4.07 | 5.22 | −2.22 | HKDC1 | 5.11 | 3.97 | 2.21 |
| MLXIP | 7.45 | 8.59 | −2.21 | ZNF790 | 7.84 | 6.7 | 2.21 |
| SPATA5 | 5.56 | 6.7 | −2.21 | DEC1 | 5.01 | 3.86 | 2.22 |
| IL12A | 5.87 | 7.01 | −2.2 | IGF1 | 6.59 | 5.44 | 2.22 |
| OR2Y1 | 3.33 | 4.47 | −2.2 | UNC119B | 5.11 | 3.94 | 2.25 |
| ZNF606 | 6.7 | 7.84 | −2.2 | ACKR3 | 6.91 | 5.72 | 2.28 |
| TBC1D3K | 7.23 | 8.36 | −2.19 | PDK4 | 4.72 | 3.53 | 2.28 |
| SH3GL3 | 4.24 | 5.36 | −2.16 | GRK4 | 6.09 | 4.88 | 2.3 |
| ID1 | 5.29 | 6.4 | −2.15 | METTL7B | 7.45 | 6.25 | 2.3 |
| ITGAM | 3.87 | 4.97 | −2.14 | TMEM25 | 8.16 | 6.95 | 2.31 |
| PNPLA7 | 3.19 | 4.29 | −2.14 | SLC22A18AS | 7.81 | 6.59 | 2.33 |
| KRT23 | 4.56 | 5.66 | −2.14 | OR52E1 | 5.78 | 4.55 | 2.35 |
| HADHA | 4.6 | 5.69 | −2.13 | EFHC2 | 4.61 | 3.37 | 2.36 |
| TCERG1 | 4.02 | 5.11 | −2.13 | GAGE2D | 4.81 | 3.57 | 2.37 |
| GALNT14 | 4.75 | 5.84 | −2.13 | IL23R | 4.41 | 3.16 | 2.37 |
| DDX11 | 5.3 | 6.38 | −2.11 | RADIL | 4.6 | 3.36 | 2.38 |
| PPAP2B | 4.98 | 6.04 | −2.09 | THSD4 | 6.02 | 4.76 | 2.4 |
| ATP13A3 | 5.33 | 6.39 | −2.09 | FAM53B | 7.19 | 5.92 | 2.4 |
| PPM1L | 4.38 | 5.44 | −2.09 | TRIM31 | 5.27 | 4 | 2.42 |
| CCKBR | 6.32 | 7.38 | −2.08 | SEMA3A | 4.29 | 3.01 | 2.43 |
| PRELID3A | 5.97 | 7.03 | −2.08 | PLCB1 | 5.66 | 4.35 | 2.49 |
| TBC1D3G | 7.84 | 8.9 | −2.08 | GRIK2 | 7.07 | 5.76 | 2.49 |
| ZNF721 | 7.83 | 8.88 | −2.08 | PPP4R4 | 5.47 | 4.13 | 2.53 |
| ZNF536 | 4.31 | 5.36 | −2.08 | CCDC173 | 5.72 | 4.38 | 2.54 |
| MLXIP | 5.4 | 6.46 | −2.07 | CLYBL | 5.98 | 4.54 | 2.72 |
| CYP2R1 | 3.66 | 4.71 | −2.07 | SERINC5 | 5.59 | 4.09 | 2.84 |
| PTPN13 | 4.77 | 5.81 | −2.07 | OR2B6 | 5.85 | 4.34 | 2.85 |
| PRICKLE3 | 6.18 | 7.23 | −2.06 | AFF4 | 7.14 | 5.63 | 2.87 |
| DISP2 | 4.28 | 5.32 | −2.06 | PPM1B | 5.93 | 4.33 | 3.03 |
| XKR9 | 4.9 | 5.93 | −2.05 | LAP3 | 5.87 | 4.28 | 3.03 |
| SNX5 | 6.28 | 7.32 | −2.05 | OR9K2 | 8.03 | 6.19 | 3.57 |
| IL15 | 6.55 | 7.58 | −2.05 | | | | |
| OR8K5 | 3.55 | 4.58 | −2.04 | | | | |
| ATP2A3 | 8.33 | 9.36 | −2.04 | | | | |
| YWHAG | 5.33 | 6.35 | −2.03 | | | | |
| CCDC84 | 4.87 | 5.89 | −2.03 | | | | |
| TAS2R4 | 4.48 | 5.5 | −2.03 | | | | |
| TEF | 6.2 | 7.22 | −2.03 | | | | |
| OR5D13 | 3.81 | 4.83 | −2.02 | | | | |
| EFHB | 3.6 | 4.61 | −2.02 | | | | |
| ELF1 | 4.28 | 5.3 | −2.02 | | | | |
| NR2C2 | 5.61 | 6.62 | −2.02 | | | | |
| ITLN2 | 3.41 | 4.42 | −2.01 | | | | |
| JSRP1 | 3.38 | 4.39 | −2.01 | | | | |
| COL27A1 | 7.15 | 8.16 | −2.01 | | | | |
| TRIM27 | 4.16 | 5.16 | −2.01 | | | | |
| HSFX2 | 6.64 | 7.64 | −2 | | | | |
| MEDAG | 4.4 | 5.4 | −2 | | | | |

TABLE 7
Differentiation of gene expression of human corneal keratocytes after 24 hours of incubation
with human C3a/C5a 0.1 μg/ml and human C3a/C5a 0.1 μg/ml with human C5L2 0.3 μg/ml.

| | C3a/C5a | C3a/C5a/ | | | C3a/C5a | C3a/C5a/ | |
|---|---|---|---|---|---|---|---|
| Gene | Avg | C5L2 Avg | Fold | Gene | Avg | C5L2 Avg | Fold |
| Symbol | (log2) | (log2) | Change | Symbol | (log2) | (log2) | Change |
| ZNF512 | 4.99 | 6.44 | −2.73 | KBTBD8 | 6.84 | 5.83 | 2 |
| HYOU1 | 3.64 | 5.05 | −2.66 | SLC8A2 | 7.33 | 6.32 | 2.01 |
| DHX35 | 3.59 | 4.99 | −2.64 | STKLD1 | 6.46 | 5.45 | 2.01 |
| TAF1 | 4.2 | 5.57 | −2.58 | KRTAP5-8 | 5.4 | 4.39 | 2.01 |
| SRP72 | 4.69 | 6 | −2.47 | KPNA3 | 7.83 | 6.82 | 2.02 |
| MIR4738 | 3.78 | 5.07 | −2.45 | CFAP53 | 4.78 | 3.76 | 2.02 |
| SELL | 3.71 | 4.98 | −2.41 | CCDC85A | 4.71 | 3.69 | 2.02 |
| SLC12A9 | 6.8 | 8.07 | −2.41 | CCDC33 | 9.42 | 8.4 | 2.03 |
| SPX | 4.33 | 5.56 | −2.35 | SCUBE1 | 7.27 | 6.24 | 2.04 |
| ATP8A2 | 3.94 | 5.14 | −2.29 | POTEM | 4.66 | 3.63 | 2.04 |
| SLC38A9 | 5.44 | 6.59 | −2.21 | LOC79999 | 4.89 | 3.85 | 2.05 |
| FAM131B | 5.14 | 6.27 | −2.19 | HIVEP2 | 4.27 | 3.22 | 2.06 |
| MARCH9 | 6.15 | 7.27 | −2.18 | MUC12 | 6.23 | 5.18 | 2.07 |
| LOC105377348 | 4.56 | 5.68 | −2.18 | ESAM | 5.93 | 4.88 | 2.08 |
| ALKBH7 | 6.03 | 7.14 | −2.16 | DOCK3 | 5.79 | 4.74 | 2.08 |
| CPB1 | 3.11 | 4.22 | −2.16 | SYT12 | 5.42 | 4.36 | 2.08 |
| YEATS2 | 3.24 | 4.34 | −2.15 | DUS3L | 6.53 | 5.47 | 2.08 |
| MAGEE1 | 4.9 | 6 | −2.15 | OR5I1 | 4.71 | 3.65 | 2.09 |
| DFNB31 | 4.47 | 5.54 | −2.09 | IL7 | 5.07 | 4 | 2.1 |
| PSRC1 | 7.44 | 8.49 | −2.08 | GUCA1A | 5.92 | 4.85 | 2.1 |
| USP24 | 4.77 | 5.82 | −2.07 | C8orf46 | 6.35 | 5.27 | 2.11 |
| ACTR3C | 5.46 | 6.51 | −2.07 | GPR22 | 4.37 | 3.29 | 2.11 |
| PAK3 | 4.68 | 5.69 | −2.03 | CCDC110 | 4.71 | 3.63 | 2.12 |
| MPPE1 | 7.28 | 8.3 | −2.02 | OPCML | 4.66 | 3.57 | 2.12 |
| IKZF3 | 4.75 | 5.76 | −2.02 | PRKCH | 5.96 | 4.87 | 2.12 |
| KIRREL3 | 4.47 | 5.48 | −2.01 | THADA | 6.07 | 4.98 | 2.13 |
| MAK | 3.93 | 4.93 | −2.01 | PABPC4L | 7.6 | 6.5 | 2.13 |
| | | | | ARHGEF1 | 6.38 | 5.29 | 2.14 |
| | | | | OR2B2 | 4.36 | 3.27 | 2.14 |
| | | | | FCAMR | 7.12 | 6.02 | 2.14 |
| | | | | CCDC38 | 5.81 | 4.69 | 2.17 |
| | | | | PHGR1 | 6.18 | 5.05 | 2.19 |
| | | | | SLC38A8 | 7.34 | 6.19 | 2.22 |
| | | | | LIPA | 5.74 | 4.58 | 2.23 |
| | | | | OR52E6 | 5.52 | 4.35 | 2.26 |
| | | | | DSG4 | 4.68 | 3.51 | 2.26 |
| | | | | LMOD3 | 4.87 | 3.69 | 2.26 |
| | | | | DDX11 | 6.55 | 5.37 | 2.27 |
| | | | | OR14A16 | 4.56 | 3.38 | 2.28 |
| | | | | C2orf83 | 6.42 | 5.22 | 2.29 |
| | | | | LAIR1 | 5.33 | 4.13 | 2.3 |
| | | | | TAL1 | 5.78 | 4.57 | 2.3 |
| | | | | CCSER1 | 4.47 | 3.25 | 2.32 |
| | | | | HMG20A | 6.67 | 5.45 | 2.33 |
| | | | | PTHLH | 6.57 | 5.35 | 2.33 |
| | | | | ACVRL1 | 7.01 | 5.78 | 2.34 |
| | | | | TEX29 | 5.9 | 4.68 | 2.34 |
| | | | | PADI4 | 5.84 | 4.58 | 2.39 |
| | | | | SH3TC1 | 8.17 | 6.91 | 2.39 |
| | | | | DHRSX | 5.26 | 3.99 | 2.42 |
| | | | | MOBP | 7.49 | 6.2 | 2.45 |
| | | | | PLGLB2 | 5.81 | 4.51 | 2.46 |
| | | | | P2RY10 | 6 | 4.68 | 2.5 |
| | | | | TCAP | 9.99 | 8.52 | 2.76 |
| | | | | SRGN | 8.82 | 7.34 | 2.78 |

TABLE 8
Differentiation of gene expression of human corneal keratocytes after 24 hours of
incubation with human C5L2 0.3 μg/ml and serum-free medium (control).

| | C5L2 | serumfree | | | C5L2 | serumfree | |
|---|---|---|---|---|---|---|---|
| Gene | Avg | Avg | Fold | Gene | Avg | Avg | Fold |
| Symbol | (log2) | (log2) | Change | Symbol | (log2) | (log2) | Change |
| EPHA4 | 4.11 | 5.73 | −3.08 | AKR1C3 | 7.35 | 6.34 | 2 |
| PLEKHA6 | 4.46 | 6.03 | −2.97 | TTC28 | 6.57 | 5.57 | 2.01 |
| EPHA4 | 4.19 | 5.57 | −2.61 | AOC2 | 4.8 | 3.79 | 2.01 |
| SCAPER | 3.33 | 4.68 | −2.54 | FAM153A | 5.12 | 4.11 | 2.01 |
| CYP21A1P | 4.42 | 5.76 | −2.53 | PHACTR2 | 4.72 | 3.7 | 2.02 |
| SLC44A5 | 4.69 | 5.98 | −2.45 | ABCG2 | 6.25 | 5.23 | 2.02 |
| KDM1A | 3.76 | 5.03 | −2.41 | OR51B5 | 4.46 | 3.44 | 2.03 |
| SORBS2 | 3.1 | 4.37 | −2.41 | NCAM1 | 6.63 | 5.6 | 2.04 |
| MBOAT2 | 4.8 | 6.01 | −2.31 | OR4F6 | 4.24 | 3.21 | 2.05 |
| LINC00174 | 4.48 | 5.68 | −2.31 | OTOR | 5.88 | 4.85 | 2.05 |
| SKAP1 | 3.88 | 5.08 | −2.3 | EVPLL | 5.57 | 4.53 | 2.06 |
| CST9L | 3.83 | 5.01 | −2.26 | ZNF582-AS1 | 5.47 | 4.43 | 2.06 |
| SOHLH2 | 3.54 | 4.72 | −2.25 | RRP36 | 7.96 | 6.92 | 2.06 |
| ACSM1 | 3.11 | 4.28 | −2.25 | TMEM2 | 7.06 | 6.01 | 2.07 |
| PVRL4 | 6.58 | 7.73 | −2.23 | OSBPL1A | 4.23 | 3.18 | 2.07 |
| EFCAB10 | 5.59 | 6.74 | −2.23 | IKZF1 | 4.33 | 3.27 | 2.08 |
| AF131215.3 | 4.86 | 6.02 | −2.23 | DACT1 | 5.63 | 4.57 | 2.08 |
| CLEC4A | 4.78 | 5.93 | −2.22 | SPATA19 | 4.53 | 3.47 | 2.08 |
| PLA2G5 | 5.54 | 6.67 | −2.2 | FLOT2 | 6.14 | 5.08 | 2.08 |
| PDK1 | 11.02 | 12.15 | −2.19 | EFCAB1 | 5.39 | 4.33 | 2.09 |
| CGNL1 | 3.89 | 5 | −2.16 | C8orf46 | 5.87 | 4.8 | 2.09 |
| MAGEAl | 5.29 | 6.4 | −2.15 | SEMA5A | 7.18 | 6.11 | 2.1 |
| TRGJ1 | 3.47 | 4.57 | −2.14 | TSC1 | 7.5 | 6.43 | 2.1 |
| AGR2 | 3.74 | 4.84 | −2.14 | CCDC79 | 6.37 | 5.29 | 2.1 |
| USP49 | 6.45 | 7.54 | −2.13 | SLC22A5 | 7.42 | 6.34 | 2.11 |
| RPS3A | 5.91 | 7 | −2.13 | S100A7 | 4.1 | 3.02 | 2.11 |
| C20orf196 | 4.24 | 5.32 | −2.12 | LEAP2 | 5.06 | 3.98 | 2.12 |
| TJP3 | 2.99 | 4.06 | −2.11 | NSUN6 | 7.77 | 6.67 | 2.14 |
| WDR1 | 4.88 | 5.95 | −2.1 | MICALCL | 5.22 | 4.12 | 2.14 |
| CHST4 | 3.84 | 4.9 | −2.08 | RGS8 | 5.04 | 3.94 | 2.15 |
| MTM1 | 5.71 | 6.76 | −2.07 | YEATS2 | 5.58 | 4.47 | 2.15 |
| TRIM10 | 5.55 | 6.6 | −2.07 | OR5AL1 | 4.58 | 3.47 | 2.17 |
| MYO1E | 5.47 | 6.52 | −2.07 | SPARCL1 | 5.14 | 4.01 | 2.19 |
| PDE1A | 3.16 | 4.21 | −2.07 | LONRF2 | 7.1 | 5.96 | 2.2 |
| FAM160A1 | 3.76 | 4.81 | −2.06 | TCF4 | 5.75 | 4.61 | 2.2 |
| ZBTB9 | 8.23 | 9.27 | −2.06 | NLGN4Y | 4.83 | 3.68 | 2.21 |
| OR5T1 | 4.35 | 5.38 | −2.05 | IFIH1 | 8.3 | 7.15 | 2.22 |
| PGK2 | 4.66 | 5.69 | −2.05 | DIO2 | 5.38 | 4.22 | 2.24 |
| C4orf50 | 4.93 | 5.96 | −2.04 | ATXN1 | 7.7 | 6.53 | 2.24 |
| NDUFA10 | 4.22 | 5.25 | −2.04 | MX1 | 7.68 | 6.51 | 2.25 |
| FEZF2 | 4.51 | 5.53 | −2.04 | FAM182B | 6.16 | 4.98 | 2.27 |
| EVI5 | 4.96 | 5.99 | −2.04 | ELF1 | 7.08 | 5.89 | 2.28 |
| SCIMP | 4.7 | 5.72 | −2.04 | TRIM27 | 5.89 | 4.69 | 2.29 |
| GBP4 | 4.75 | 5.77 | −2.03 | GHRHR | 5.62 | 4.41 | 2.32 |
| INPP5D | 3.66 | 4.68 | −2.03 | MS4A12 | 5.83 | 4.61 | 2.33 |
| NEK5 | 4.09 | 5.11 | −2.02 | VPS8 | 4.95 | 3.73 | 2.34 |
| BNIP3 | 13.97 | 14.97 | −2.01 | PATE1 | 5.97 | 4.74 | 2.34 |
| | | | | CDRT1 | 5.85 | 4.61 | 2.36 |
| | | | | HTN1 | 4.64 | 3.4 | 2.36 |
| | | | | KCNN3 | 5.48 | 4.24 | 2.37 |
| | | | | ZNF546 | 5.49 | 4.24 | 2.39 |
| | | | | MRAS | 6.63 | 5.31 | 2.5 |
| | | | | UBE2D3 | 5.33 | 3.99 | 2.53 |
| | | | | KIRREL3 | 5.43 | 4.05 | 2.61 |
| | | | | MYEF2 | 5.67 | 4.26 | 2.66 |
| | | | | IFT44L | 5.11 | 3.66 | 2.75 |
| | | | | SLC38A9 | 7.29 | 5.83 | 2.76 |
| | | | | LAP3 | 5.59 | 3.75 | 3.58 |

TABLE 9
Differentially expressed genes in mouse corneas, 5 days after corneal alkali-burn and
treatment, between the ‘PBS/control’ and ‘PBS with mC5L2’ treatment group.

| Down-regulated genes (mC5L2 vs. PBS) | | | | Up-regulated genes (mC5L2 vs. PBS) | | | |
|---|---|---|---|---|---|---|---|
| Gene | baseMean | Fold | adjusted | Gene | baseMean | Fold | adjusted |
| Symbol | (log2) | Change | p-value | Symbol | (log2) | Change | p-value |
| Tph2 | 4.31 | −2.19 | 0.006 | Gm10318 | 6.68 | 1.00 | 0.012 |
| Scn7a | 5.59 | −2.00 | 0.033 | Gm6880 | 4.55 | 1.00 | 0.012 |
| Dsg4 | 3.00 | −1.78 | 0.006 | Klk6 | 4.23 | 1.00 | 0.027 |
| Foxp2 | 5.07 | −1.44 | 0.025 | 1700020 | 4.41 | 1.01 | 0.030 |
| | | | | D05Rik | | | |
| Itih5 | 6.81 | −1.33 | 0.037 | D6Ertd | 6.07 | 1.01 | 0.015 |
| | | | | 527e | | | |
| Pigr | 5.27 | −1.32 | 0.031 | Flna | 11.12 | 1.01 | 0.029 |
| Hnmt | 5.61 | −1.27 | 0.027 | Naf1 | 7.40 | 1.01 | 0.016 |
| Xlr3c | 3.71 | −1.23 | 0.027 | Sec31b | 5.24 | 1.01 | 0.018 |
| Slc18a1 | 5.39 | −1.23 | 0.009 | Tbx20 | 4.58 | 1.02 | 0.033 |
| Ube2dn1 | 4.61 | −1.20 | 0.022 | Prrc2a | 8.19 | 1.03 | 0.008 |
| Tnn | 4.54 | −1.20 | 0.014 | Hsd11b2 | 5.35 | 1.04 | 0.032 |
| 1700008 | 2.88 | −1.20 | 0.025 | 1700006 | 3.76 | 1.05 | 0.048 |
| P02Rik | | | | A11Rik | | | |
| Polr1e | 6.30 | −1.15 | 0.012 | Fam46c | 7.29 | 1.05 | 0.006 |
| Acsm1 | 9.62 | −1.12 | 0.016 | Defb5 | 4.71 | 1.05 | 0.023 |
| Fgg | 3.41 | −1.12 | 0.047 | Ghrhr | 4.45 | 1.05 | 0.032 |
| Gin1 | 6.38 | −1.09 | 0.016 | Ttc9b | 4.33 | 1.07 | 0.012 |
| Olfr890 | 2.13 | −1.09 | 0.032 | Ccr6 | 3.68 | 1.07 | 0.033 |
| Ldlrad3 | 7.01 | −1.08 | 0.019 | Txk | 4.87 | 1.07 | 0.035 |
| Pstk | 4.60 | −1.07 | 0.027 | Qprt | 6.48 | 1.08 | 0.012 |
| Tspan18 | 3.40 | −1.06 | 0.042 | Nxph4 | 6.25 | 1.09 | 0.015 |
| Usp32 | 6.74 | −1.04 | 0.015 | 1700013 | 4.55 | 1.13 | 0.006 |
| | | | | F07Rik | | | |
| Cd200r4 | 2.68 | −1.04 | 0.008 | Prlh | 6.72 | 1.15 | 0.012 |
| Gm5795 | 2.85 | −1.04 | 0.028 | Defb8 | 5.44 | 1.21 | 0.006 |
| 1700012 | 5.50 | −1.03 | 0.012 | Tnfrsf1a | 9.90 | 1.21 | 0.006 |
| B09Rik | | | | | | | |
| Itgb7 | 8.16 | −1.01 | 0.007 | Sprr2b | 6.78 | 1.22 | 0.015 |
| | | | | Sprr2j-ps | 7.33 | 1.22 | 0.015 |
| | | | | Atp4a | 5.26 | 1.23 | 0.012 |
| | | | | Lrrc15 | 6.52 | 1.23 | 0.031 |
| | | | | D130040 | 4.12 | 1.42 | 0.012 |
| | | | | H23Rik | | | |

TABLE 10
Differentially expressed genes in mouse corneas, 10 days after corneal alkali-burn and
treatment, between the ‘PBS/control’ and ‘PBS with mC5L2’ treatment group.

| Down-regulated genes (mC5L2 vs. PBS) | | | | Up-regulated genes (mC5L2 vs. PBS) | | | |
|---|---|---|---|---|---|---|---|
| Gene | baseMean | Fold | adjusted | Gene | baseMean | Fold | adjusted |
| Symbol | (log2) | Change | p-value | Symbol | (log2) | Change | p-value |
| Xlr4b | 6.30 | −1.72 | 0.022 | Ltbp3 | 7.78 | 1.02 | 0.021 |
| Asb11 | 6.28 | −1.57 | 0.022 | Jcad | 6.00 | 1.02 | 0.042 |
| Inpp1 | 7.35 | −1.47 | 0.021 | Tcf4 | 9.65 | 1.02 | 0.033 |
| Als2cr12 | 5.66 | −1.44 | 0.022 | Pcdhb20 | 5.25 | 1.03 | 0.037 |
| Ddx60 | 7.77 | −1.40 | 0.025 | Grasp | 6.83 | 1.03 | 0.034 |
| Cldn17 | 6.18 | −1.36 | 0.033 | Nfatc4 | 6.20 | 1.03 | 0.022 |
| St8sia6 | 7.51 | −1.35 | 0.036 | Cacna1g | 6.48 | 1.03 | 0.039 |
| Tmprss11d | 8.64 | −1.35 | 0.021 | Adamts10 | 6.65 | 1.03 | 0.026 |
| Capsl | 5.51 | −1.31 | 0.039 | Reck | 5.22 | 1.03 | 0.045 |
| Adh6a | 9.21 | −1.31 | 0.046 | Serpinf1 | 9.20 | 1.04 | 0.024 |
| Prdm1 | 7.96 | −1.30 | 0.045 | Slc41a2 | 5.46 | 1.04 | 0.041 |
| Hpgds | 9.62 | −1.30 | 0.024 | Sprr2j-ps | 7.33 | 1.04 | 0.028 |
| Sdr9c7 | 6.75 | −1.29 | 0.021 | Rnd1 | 4.30 | 1.05 | 0.046 |
| Arhgef37 | 6.94 | −1.28 | 0.021 | Bace1 | 6.26 | 1.06 | 0.022 |
| Gm7008 | 4.69 | −1.26 | 0.021 | Gpc6 | 6.35 | 1.07 | 0.042 |
| Slco4c1 | 5.52 | −1.26 | 0.037 | Tns1 | 7.91 | 1.07 | 0.024 |
| Rnf39 | 8.83 | −1.25 | 0.024 | Itpripl2 | 6.69 | 1.07 | 0.041 |
| 2310009 | 5.18 | −1.24 | 0.021 | Fndc3b | 9.11 | 1.08 | 0.028 |
| B15Rik | | | | | | | |
| Serpinb8 | 7.96 | −1.23 | 0.021 | Fkbp9 | 9.45 | 1.10 | 0.033 |
| Id2 | 9.97 | −1.23 | 0.048 | Zfp423 | 6.57 | 1.10 | 0.031 |
| Tuft1 | 8.18 | −1.23 | 0.021 | Lrp1 | 9.72 | 1.11 | 0.041 |
| Tmem159 | 8.06 | −1.23 | 0.041 | Plxna4 | 5.43 | 1.12 | 0.042 |
| Ace2 | 6.16 | −1.20 | 0.043 | Gaa | 9.44 | 1.12 | 0.022 |
| Nabp1 | 9.16 | −1.19 | 0.021 | Cstad | 5.76 | 1.12 | 0.022 |
| Alpk3 | 4.34 | −1.18 | 0.037 | Flna | 11.12 | 1.13 | 0.024 |
| Usp32 | 6.74 | −1.17 | 0.021 | Tenm3 | 7.55 | 1.16 | 0.022 |
| Pigr | 5.27 | −1.15 | 0.049 | H2afx | 9.28 | 1.16 | 0.022 |
| Pir | 8.13 | −1.14 | 0.021 | Alox5 | 5.61 | 1.17 | 0.024 |
| Dnajb4 | 8.25 | −1.14 | 0.040 | Tsku | 7.14 | 1.17 | 0.023 |
| Ppfibp2 | 4.25 | −1.13 | 0.048 | Tgfb1i1 | 6.67 | 1.19 | 0.035 |
| Mettl5 | 6.49 | −1.13 | 0.024 | Fgfr1 | 6.86 | 1.20 | 0.045 |
| Lrrc31 | 4.72 | −1.12 | 0.047 | Ext1 | 8.95 | 1.21 | 0.031 |
| Cd274 | 7.94 | −1.10 | 0.047 | Adamts2 | 7.98 | 1.22 | 0.034 |
| Lipm | 7.61 | −1.09 | 0.039 | Slc39a14 | 7.04 | 1.22 | 0.045 |
| Bcas1 | 8.49 | −1.07 | 0.041 | Lrrc15 | 6.52 | 1.22 | 0.037 |
| D18r1 | 6.88 | −1.07 | 0.031 | Chpf | 7.58 | 1.26 | 0.024 |
| Cmc1 | 6.72 | −1.06 | 0.044 | Scarf2 | 8.02 | 1.26 | 0.044 |
| Edn1 | 5.86 | −1.06 | 0.041 | Slit3 | 8.33 | 1.29 | 0.034 |
| Casp14 | 5.23 | −1.06 | 0.031 | Col5a2 | 7.42 | 1.29 | 0.038 |
| Kctd9 | 8.45 | −1.04 | 0.021 | Apbb2 | 9.67 | 1.29 | 0.041 |
| Cirbp | 7.05 | −1.04 | 0.050 | Fkbp10 | 7.83 | 1.31 | 0.046 |
| Ociad2 | 6.22 | −1.04 | 0.022 | Col5a1 | 7.39 | 1.32 | 0.047 |
| Mr1 | 8.69 | −1.02 | 0.033 | Nlgn2 | 6.79 | 1.32 | 0.045 |
| Oas12 | 9.63 | −1.02 | 0.028 | Kirrel | 6.98 | 1.32 | 0.050 |
| Usp54 | 8.11 | −1.01 | 0.021 | Gprl53 | 5.80 | 1.35 | 0.028 |
| Zfp772 | 7.60 | −1.01 | 0.033 | Dpysl3 | 7.93 | 1.37 | 0.039 |
| Slc17a5 | 8.05 | −1.00 | 0.037 | Sulf1 | 8.37 | 1.38 | 0.047 |
| | | | | Tgfb1 | 8.42 | 1.38 | 0.024 |
| | | | | Tfrc | 6.74 | 1.44 | 0.024 |
| | | | | Tnfrsf1a | 9.90 | 1.45 | 0.001 |
| | | | | Dchs1 | 7.28 | 1.45 | 0.026 |
| | | | | Colla1 | 11.22 | 1.49 | 0.024 |
| | | | | Mrc2 | 8.75 | 1.53 | 0.029 |
| | | | | Itga11 | 6.68 | 1.53 | 0.048 |
| | | | | Tagln | 7.05 | 1.56 | 0.035 |
| | | | | Steap1 | 5.82 | 1.57 | 0.036 |
| | | | | Serpine2 | 6.68 | 1.58 | 0.021 |
| | | | | Tnfaip2 | 7.75 | 1.61 | 0.042 |
| | | | | Pdgfrb | 8.15 | 1.63 | 0.047 |
| | | | | Ncam1 | 8.10 | 1.67 | 0.033 |
| | | | | Thbs1 | 10.84 | 1.68 | 0.047 |
| | | | | Bgn | 10.83 | 1.69 | 0.031 |
| | | | | Tmem47 | 8.22 | 1.70 | 0.033 |
| | | | | Tgfb3 | 7.63 | 1.71 | 0.024 |
| | | | | Mmp2 | 10.57 | 1.73 | 0.046 |
| | | | | Pmepa1 | 8.27 | 1.74 | 0.030 |
| | | | | Hspg2 | 7.53 | 1.74 | 0.026 |
| | | | | Pitx2 | 8.23 | 1.75 | 0.024 |
| | | | | Sdc3 | 8.26 | 1.75 | 0.050 |
| | | | | Creb311 | 6.81 | 1.79 | 0.038 |
| | | | | Fbn1 | 7.26 | 1.80 | 0.045 |
| | | | | Fbln5 | 9.36 | 1.90 | 0.031 |
| | | | | Laptm5 | 7.77 | 1.92 | 0.049 |
| | | | | Lox11 | 7.85 | 1.95 | 0.037 |
| | | | | Lrrc32 | 6.20 | 1.99 | 0.037 |
| | | | | Eng | 8.04 | 2.01 | 0.031 |
| | | | | Prelp | 9.60 | 2.04 | 0.031 |
| | | | | Fmod | 10.49 | 2.18 | 0.042 |
| | | | | Igsf10 | 6.87 | 2.22 | 0.048 |
| | | | | Aebp1 | 9.74 | 2.25 | 0.031 |
| | | | | Gpx3 | 8.12 | 2.25 | 0.021 |
| | | | | Sod3 | 8.82 | 2.50 | 0.025 |

TABLE 11
Differentially expressed genes in mouse corneas, 20 days after corneal alkali-burn and
treatment, between the ‘PBS/control’ and ‘PBS with mC5L2’ treatment group.

| Down-regulated genes (mC5L2 vs. PBS) | | | | Up-regulated genes (mC5L2 vs. PBS) | | | |
|---|---|---|---|---|---|---|---|
| Gene | baseMean | Fold | adjusted | Gene | baseMean | Fold | adjusted |
| Symbol | (log2) | Change | p-value | Symbol | (log2) | Change | p-value |
| Arntl | 6.02 | −1.75 | 0.008 | Fxyd3 | 10.09 | 1.02 | 0.031 |
| Neil3 | 6.47 | −1.19 | 0.042 | Lim2 | 5.33 | 1.03 | 0.031 |
| Asns | 8.01 | −1.11 | 0.039 | Arhgap27 | 8.39 | 1.05 | 0.008 |
| Adprh | 5.95 | −1.04 | 0.031 | Cfap100 | 7.35 | 1.05 | 0.031 |
| | | | | Ciart | 5.13 | 1.11 | 0.049 |
| | | | | Per2 | 7.27 | 1.63 | 0.008 |

TABLE 12
Functional annotations of differentially expressed genes in mouse corneas, 10 days
after corneal alkali-burn and treatment, between the ‘PBS/control’ and ‘PBS with mC5L2’
treatment group (trunked to the 100 most significant).

| | | adj. p- | |
|---|---|---|---|
| Accession | Description | value | Gene Symbols |
| GO:0031012 | extracellular matrix | <0.001 | Fmod/Col5a2/Serpine2/Prelp/Col5a1/Thbs1/Fbn1/ |
| | | | Sdc3/Hspg2/Sod3/Tgfb1/Tgfbli1/Mmp2/Ncam1/ |
| | | | Loxl1/Bgn/Aebp1/Slit3/Adamts2/Col1a1/Serpinf1/ |
| | | | Tgfb3/Fbln5/Gpc6/Lrrc15/Adamts10/Ltbp3 |
| GO:0005578 | proteinaceous | <0.001 | Fmod/Col5a2/Prelp/Col5a1/Fbn1/Hspg2/Tgfb1/ |
| | extracellular matrix | | Mmp2/Loxl1/Bgn/Slit3/Adamts2/Col1a1/Serpinf1/ |
| | | | Tgfb3/Fbln5/Gpc6/Adamts10/Ltbp3 |
| GO:0030198 | extracellular matrix | <0.001 | Sulf1/Col5a2/Col5a1/Thbs1/Creb311/Eng/Reck/ |
| | organization | | Hspg2/Apbb2/Tgfb1/Lox11/Adamts2/Col1a1/Fbln5 |
| GO:0032963 | collagen metabolic | <0.001 | Col5a1/Creb311/Eng/Tgfb1/Mmp2/Adamts2/ |
| | process | | Col1a1/Mrc2/Tgfb3/Pdgfrb |
| GO:0043062 | extracellular structure | <0.001 | Sulf1/Col5a2/Col5al/Thbs1/Creb311/Eng/Reck/ |
| | organization | | Hspg2/Apbb2/Tgfb1/Loxl1/Adamts2/Col1a1/Fbln5 |
| GO:0032964 | collagen biosynthetic | <0.001 | Col5al/Creb311/Eng/Tgfb1/Col1a1/Tgfb3/Pdgfrb |
| | process | | |
| GO:0001501 | skeletal system | <0.001 | Sulf1/Col5a2/Thbs1/Fbn1/Pitx2/Hspg2/Tgfb1/ |
| | development | | Dchs1/Fgfr1/Mmp2/Col1a1/Tgfb3/Edn1/Ext1/ |
| | | | Pdgfrb/Ltbp3 |
| GO:0001525 | angiogenesis | <0.001 | Sulf1/Thbs1/Eng/Pitx2/Hspg2/Tnfrsf1a/Fgfr1/ |
| | | | Mmp2/Flna/Serpinf1/Tnfaip2/Edn1/Nfatc4/Jcad/ |
| | | | Pdgfrb/Tcf4 |
| GO:0090287 | reg. of cellular resp. to | <0.001 | Sulf1/Thbs1/Fbn1/Pmepa1/Eng/Tgfb1/Tgfb1i1/ |
| | growth factor stimulus | | Fgfr1/Zfp423/Tgfb3/Jcad/Tcf4 |
| GO:0044420 | extracellular matrix | <0.001 | Col5a2/Col5al/Fbn1/Hspg2/Loxl1/Col1a1/Serpinf1/ |
| | component | | Fbln5/Adamts10 |
| GO:0061448 | connective tissue | <0.001 | Sulf1/Col5al/Thbs1/Hspg2/Tgfb1/Fgfr1/Col1a1/ |
| | development | | Id2/Edn1/Pdgfrb/Ltbp3 |
| GO:0005539 | glycosaminoglycan | <0.001 | Serpine2/Prelp/Col5a1/Thbs1/Fbn1/Eng/Fgfr1/ |
| | binding | | Ncam1/Bgn/Dpysl3 |
| GO:0060485 | mesenchyme | <0.001 | Thbs1/Eng/Pitx2/Tgfb1/Tgfb1i1/Dchs1/Fgfr1/ |
| | development | | Flna/Col1a1/Tgfb3/Edn1 |
| GO:0032967 | pos. reg. of collagen | <0.001 | Creb311/Eng/Tgfb1/Tgfb3/Pdgfrb |
| | biosynthetic process | | |
| GO:0001503 | ossification | <0.001 | Creb311/Fndc3b/Igsf10/Hspg2/Tgfb1/Dchs1/Fgfr1/ |
| | | | Mmp2/Col1a1/Id2/Ext1/Ltbp3 |
| GO:0010714 | pos. reg. of collagen | <0.001 | Creb311/Eng/Tgfb1/Tgfb3/Pdgfrb |
| | metabolic process | | |
| GO:0017015 | reg. of transforming | <0.001 | Thbs1/Fbn1/Pmepa1/Eng/Tgfb1/Tgfb1i1/Tgfb3 |
| | growth factor beta | | |
| | receptor sign, pathway | | |
| GO: 1903844 | reg. of cellular resp. to | <0.001 | Thbs1/Fbn1/Pmepa1/Eng/Tgfb1/Tgfb1i1/Tgfb3 |
| | transforming growth | | |
| | factor beta stimulus | | |
| GO:0048738 | cardiac muscle tissue | <0.001 | Eng/Pitx2/Hspg2/Tgfb1/Alpk3/Fgfr1/Ncam1/Id2/ |
| | development | | Edn1/Pdgfrb |
| GO:0060973 | cell migration involved | <0.001 | Eng/Pitx2/Dchs1/Pdgfrb |
| | in heart development | | |
| GO:0019838 | growth factor binding | 0.001 | Col5a1/Thbs1/Eng/Fgfr1/Col1a1/Tgfb3/Pdgfrb/ |
| | | | Ltbp3 |
| GO:0090100 | pos. reg. of | 0.001 | Sulf1/Thbs1/Eng/Tgfb1/Tgfb1i1/Zfp423/Tgfb3 |
| | transmembrane receptor | | |
| | protein serine/threonine | | |
| | kinase sign, pathway | | |
| GO:0007179 | transforming growth | 0.001 | Thbs1/Fbn1/Pmepa1/Eng/Tgfb1/Tgfb1i1/Tgfb3/ |
| | factor beta receptor sign, | | Ltbp3 |
| | pathway | | |
| GO:0048762 | mesenchymal cell | 0.001 | Eng/Pitx2/Tgfb1/Tgfb1i1/Fgfr1/Flna/Col1a1/ |
| | differentiation | | Tgfb3/Edn1 |
| GO:0032965 | reg. of collagen | 0.001 | Creb31l/Eng/Tgfb1/Tgfb3/Pdgfrb |
| | biosynthetic process | | |
| GO:0050431 | transforming growth | 0.001 | Thbs1/Eng/Tgfb3/Ltbp3 |
| | factor beta binding | | |
| GO:0033002 | muscle cell proliferation | 0.001 | Thbs1/Fgfr1/Mmp2/Ncam1/Ace2/Id2/Tgfb3/ |
| | | | Edn1/Pdgfrb |
| GO:0005201 | extracellular matrix | 0.001 | Col5a2/Prelp/Col5a1/Fbn1/Col1a1 |
| | structural constituent | | |
| GO:0010718 | pos. reg. of epithelial to | 0.001 | Eng/Tgfb1/Tgfb1i1/Col1a1/Tgfb3 |
| | mesenchymal transition | | |
| GO:0030199 | collagen fibril | 0.001 | Col5a2/Col5al/Loxl1/Adamts2/Col1a1 |
| | organization | | |
| GO:0090092 | reg. of transmembrane | 0.001 | Sulf1/Thbs1/Fbn1/Pmepa1/Eng/Tgfb1/Tgfb1i1/ |
| | receptor protein | | Zfp423/Tgfb3 |
| | serine/threonine kinase | | |
| | sign, pathway | | |
| GO:0010712 | reg. of collagen | 0.001 | Creb311/Eng/Tgfb1/Tgfb3/Pdgfrb |
| | metabolic process | | |
| GO:1901681 | sulfur compound | 0.002 | Serpine2/Prelp/Col5a1/Thbs1/Fbn1/Fgfr1/Ncam1/ |
| | binding | | Gpc6/Dpysl3 |
| GO:0001763 | morphogenesis of a | 0.002 | Sulf1/Eng/Pitx2/Tgfb1/Dchs1/Fgfr1/Prdm1/Edn1/ |
| | branching structure | | Nfatc4 |
| GO:0051216 | cartilage development | 0.002 | Sulf1/Thbs1/Hspg2/Tgfb1/Fgfr1/Col1a1/Edn1/ |
| | | | Ltbp3 |
| GO:0071560 | cellular resp. to | 0.002 | Thbs1/Fbn1/Pmepa1/Eng/Tgfb1/Tgfb1i1/Tgfb3/ |
| | transforming growth | | Ltbp3 |
| | factor beta stimulus | | |
| GO:0071559 | resp. to transforming | 0.002 | Thbs1/Fbn1/Pmepa1/Eng/Tgfb1/Tgfb1i1/Tgfb3/ |
| | growth factor beta | | Ltbp3 |
| GO:0007178 | transmembrane receptor | 0.002 | Sulf1/Thbs1/Fbn1/Pmepa1/Eng/Tgfb1/Tgfb1i1/ |
| | protein serine/threonine | | Zfp423/Tgfb3/Ltbp3 |
| | kinase sign, pathway | | |
| GO:0034713 | type I transforming | 0.002 | Eng/Tgfb1/Tgfb3 |
| | growth factor beta | | |
| | receptor binding | | |
| GO:0060348 | bone development | 0.002 | Sulf1/Thbs1/Fbn1/Pitx2/Hspg2/Dchs1/Col1a1/ |
| | | | Ltbp3 |
| GO:0048660 | reg. of smooth muscle | 0.002 | Thbs1/Mmp2/Ace2/Id2/Tgfb3/Edn1/Pdgfrb |
| | cell proliferation | | |
| GO:0003007 | heart morphogenesis | 0.002 | Col5a1/Thbs1/Eng/Pitx2/Tgfb1/Dchs1/Flna/Gaa/ |
| | | | Id2 |
| GO:0007162 | neg. reg. of cell | 0.003 | Serpine2/Thbs1/Plxna4/Tgfb1/Lrrc32/Mmp2/ |
| | adhesion | | Col1a1/Rnd1/Cd274 |
| GO:0008201 | heparin binding | 0.003 | Serpine2/Prelp/Col5a1/Thbs1/Fbn1/Fgfr1/Ncam1 |
| GO:0048659 | smooth muscle cell | 0.003 | Thbs1/Mmp2/Ace2/Id2/Tgfb3/Edn1/Pdgfrb |
| | proliferation | | |
| GO:0005583 | fibrillar collagen trimer | 0.003 | Col5a2/Col5a1/Col1a1 |
| GO:0048407 | platelet-derived growth | 0.003 | Col5al/Col1a1/Pdgfrb |
| | factor binding | | |
| GO:0098643 | banded collagen fibril | 0.003 | Col5a2/Col5a1/Col1a1 |
| GO:0051145 | smooth muscle cell | 0.004 | Eng/Pitx2/Tgfb1/Nfatc4/Pdgfrb |
| | differentiation | | |
| GO:0061138 | morphogenesis of a | 0.005 | Sulf1/Eng/Pitx2/Tgfb1/Dchs1/Fgfr1/Edn1/Nfatc4 |
| | branching epithelium | | |
| GO:0060325 | face morphogenesis | 0.005 | Tgfb1/Mmp2/Col1a1/Tgfb3 |
| GO:0046332 | SMAD binding | 0.005 | Col5a2/Creb311/Pmepa1/Tgfb1i1/Flna |
| GO:0006024 | glycosaminoglycan | 0.006 | Chpf/Tgfb1/Ext1/Pdgfrb |
| | biosynthetic process | | |
| GO:0030279 | neg. reg. of ossification | 0.007 | Fndc3b/Tgfb1/Fgfr1/Id2/Ltbp3 |
| GO:0098644 | complex of collagen | 0.007 | Col5a2/Col5a1/Col1a1 |
| | trimers | | |
| GO:0030336 | neg. reg. of cell | 0.007 | Sulf1/Thbs1/Eng/Reck/Tgfb1/Lrp1/Serpinf1/ |
| | migration | | Dpysl3 |
| GO:0001569 | branching involved in | 0.007 | Eng/Pitx2/Edn1/Nfatc4 |
| | blood vessel | | |
| | morphogenesis | | |
| GO:0060323 | head morphogenesis | 0.008 | Tgfb1/Mmp2/Col1a1/Tgfb3 |
| GO:0001837 | epithelial to | 0.008 | Eng/Tgfb1/Tgfb1i1/Flna/Col1a1/Tgfb3 |
| | mesenchymal transition | | |
| GO:0030203 | glycosaminoglycan | 0.008 | Chpf/Tgfb1/Bgn/Ext1/Pdgfrb |
| | metabolic process | | |
| GO:0090288 | neg. reg. of cellular resp. | 0.009 | Sulf1/Thbs1/Fbn1/Pmepa1/Tgfb1i1/Tgfb3 |
| | to growth factor | | |
| | stimulus | | |
| GO:0010717 | reg. of epithelial to | 0.009 | Eng/Tgfb1/Tgfb1i1/Col1a1/Tgfb3 |
| | mesenchymal transition | | |
| GO:0003170 | heart valve development | 0.009 | Pitx2/Tgfb1/Dchs1/Prdm1 |
| GO:0010763 | pos. reg. of fibroblast | 0.009 | Thbs1/Tgfb1/Fgfr1 |
| | migration | | |
| GO:2000146 | neg. reg. of cell motility | 0.009 | Sulf1/Thbs1/Eng/Reck/Tgfb1/Lrp1/Serpinf1/ |
| | | | Dpysl3 |
| GO:0010761 | fibroblast migration | 0.01 | Tns1/Thbs1/Tgfb1/Fgfr1 |
| GO:0001655 | urogenital system | 0.01 | Sulf1/Fbn1/Tgfb1/Dchs1/Fgfr1/Mmp2/Serpinf1/ |
| | development | | Id2/Pdgfrb |
| GO:0006023 | aminoglycan | 0.01 | Chpf/Tgfb1/Ext1/Pdgfrb |
| | biosynthetic process | | |
| GO:0055025 | pos. reg. of cardiac | 0.01 | Tgfb1/Fgfr1/Ncam1/Edn1 |
| | muscle tissue | | |
| | development | | |
| GO:0001570 | vasculogenesis | 0.01 | Eng/Pitx2/Tgfb1/Fgfr1/Pdgfrb |
| GO:0014706 | striated muscle tissue | 0.01 | Eng/Pitx2/Hspg2/Tgfb1/Alpk3/Fgfr1/Ncam1/Id2/ |
| | development | | Edn1/Pdgfrb |
| GO:0030324 | lung development | 0.011 | Pitx2/Fndc3b/Fgfr1/Adamts2/Tgfb3/Pdgfrb/ |
| | | | Ltbp3 |
| GO:0030335 | pos. reg. of cell | 0.011 | Thbs1/Tgfb1/Fgfr1/Mmp2/Flna/Col1a1/Edn1/ |
| | migration | | Lrrc15/Jcad/Pdgfrb/Cd274 |
| GO:0030323 | respiratory tube | 0.012 | Pitx2/Fndc3b/Fgfr1/Adamts2/Tgfb3/Pdgfrb/ |
| | development | | Ltbp3 |
| GO:0043536 | pos. reg. of blood vessel | 0.012 | Thbs1/Tgfb1/Fgfr1/Jcad |
| | endothelial cell | | |
| | migration | | |
| GO:0048661 | pos. reg. of smooth | 0.013 | Thbs1/Mmp2/Id2/Edn1/Pdgfrb |
| | muscle cell proliferation | | |
| GO:0009611 | resp. to wounding | 0.013 | Serpine2/Col5a1/Thbs1/Eng/Igsf10/Tgfb1/Mmp2/ |
| | | | Hna/Col1a1/Dpysl3 |
| GO:0060537 | muscle tissue | 0.014 | Eng/Pitx2/Hspg2/Tgfb1/Alpk3/Fgfr1/Ncam1/Id2/ |
| | development | | Edn1/Pdgfrb |
| GO:2000147 | pos. reg. of cell motility | 0.014 | Thbs1/Tgfb1/Fgfr1/Mmp2/Flna/Col1a1/Edn1/ |
| | | | Lrrc15/Jcad/Pdgfrb/Cd274 |
| GO:0002062 | chondrocyte | 0.014 | Sulf1/Hspg2/Tgfb1/Fgfr1/Ltbp3 |
| | differentiation | | |
| GO:0051271 | neg. reg. of cellular | 0.015 | Sulf1/Thbs1/Eng/Reck/Tgfb1/Lrp1/Serpinf1/ |
| | component movement | | Dpys13 |
| GO:0005604 | basement membrane | 0.016 | Col5a1/Fbn1/Hspg2/Loxl1/Serpinf1 |
| GO:0006022 | aminoglycan metabolic | 0.016 | Chpf/Tgfb1/Bgn/Ext1/Pdgfrb |
| | process | | |
| GO:0090596 | sensory organ | 0.016 | Col5a2/Col5al/Pitx2/Tsku/Fgfr1/Tenm3/Prdm1/ |
| | morphogenesis | | Edn1 |
| GO:0060324 | face development | 0.017 | Tgfb1/Mmp2/Col1a1/Tgfb3 |
| GO:0030574 | collagen catabolic | 0.017 | Mmp2/Adamts2/Mrc2 |
| | process | | |
| GO:0090101 | neg. reg. of | 0.017 | Fbn1/Pmepa1/Eng/Tgfb1i1/Tgfb3 |
| | transmembrane receptor | | |
| | protein serine/threonine | | |
| | kinase sign, pathway | | |
| GO:0010038 | resp. to metal ion | 0.017 | Thbs1/Sod3/Ncam1/Cacna1g/Id2/Nfatc4/Tfrc |
| GO:0006801 | superoxide metabolic | 0.017 | Sod3/Tgfb1/Fbln5/Edn1 |
| | process | | |
| GO:0030512 | neg. reg. of transforming | 0.017 | Fbn1/Pmepa1/Tgfb1i1/Tgfb3 |
| | growth factor beta | | |
| | receptor sign, pathway | | |
| GO:0035904 | aorta development | 0.017 | Eng/Prdm1/Lrp1/Pdgfrb |
| GO:0030509 | BMP sign, pathway | 0.017 | Sulf1/Fbn1/Eng/Tgfb1/Zfp423/Tgfb3 |
| GO:0010171 | body morphogenesis | 0.017 | Tgfb1/Mmp2/Col1a1/Tgfb3 |
| GO:0038084 | vascular endothelial | 0.017 | Jcad/Pdgfrb/Tcf4 |
| | growth factor sign, | | |
| | pathway | | |
| GO:0045992 | neg. reg. of embryonic | 0.017 | Sulf1/Col5a2/Col5al |
| | development | | |
| GO:0001818 | neg. reg. of cytokine | 0.017 | Thbs1/Tnfrsf1a/Tgfb1/Lrrc32/Fgfr1/Tgfb3/ |
| | production | | Cd274 |
| GO:0043235 | receptor complex | 0.017 | Pigr/Eng/Tnfrsf1a/Plxna4/Fgfr1/Itgal1/Lrp1/ |
| | | | Tfrc/Pdgfrb |
| GO: 1903522 | reg. of blood circulation | 0.018 | Alox5/Mmp2/Ace2/Flna/Gaa/Cacna1g/Edn1 |
| GO: 1903845 | neg. reg. of cellular resp. | 0.018 | Fbn1/Pmepa1/Tgfb1i1/Tgfb3 |
| | to transforming growth | | |
| | factor beta stimulus | | |
| GO:0019955 | cytokine binding | 0.018 | Thbs1/Eng/Tnfrsfla/Tgfb3/Ltbp3 |

TABLE 13
Differentially expressed proteins in mouse corneas, 20 days after corneal alkali-burn
and treatment, between the ‘PBS/control’ and ‘PBS with mC5L2’ treatment group.

| Up-regulated proteins in PBS/control group | | | | | Up-regulated genes in mC5L2 group | | | | |
|---|---|---|---|---|---|---|---|---|---|
| Protein | | | AvExp | adj. | Protein | | | AvExp | adj. |
| Symbol | UniProt | logFC | (log2) | P-val. | Symbol | UniProt | logFC | (log2) | P-val. |
| CATD | P07339 | 1.09 | 12.82 | 0.012 | MP2K4 | P45985 | T0006 | −0.52 | 10.92 |
| NCOR1 | O75376 | 1.07 | 13.78 | 0.001 | MK14 | Q16539 | T0763 | −0.52 | 10.82 |
| CATB | P07858 | 1.00 | 12.27 | 0.018 | ITAL | P20701 | S0264 | −0.52 | 12.79 |
| CD53 | P19397 | 1.00 | 14.87 | 0.002 | CD7 | P09564 | S0248 | −0.53 | 13.58 |
| NCOR1 | O75376 | 0.97 | 12.64 | 0.002 | FOLR1 | P15328 | T0670 | −0.54 | 9.99 |
| GSTM1 | P09488 | 0.84 | 10.47 | 0.000 | PPIA | P62937 | S0027 | −0.54 | 12.36 |
| CD53 | P19397 | 0.83 | 12.10 | 0.001 | CD22 | P20273 | S0292 | −0.54 | 12.37 |
| MMP1 | P03956 | 0.81 | 11.90 | 0.002 | HLA-DR | | S0373 | −0.54 | 11.78 |
| NDF6 | Q96NK8 | 0.81 | 11.62 | 0.006 | ITAL | P20701 | S0263 | −0.55 | 12.84 |
| FABP5 | Q01469 | 0.77 | 14.69 | 0.048 | PIR | O00625 | T0533 | −0.56 | 13.04 |
| GSTM3 | P21266 | 0.65 | 11.79 | 0.010 | FCG3A | P08637 | S0276 | −0.57 | 12.41 |
| Fc | | 0.65 | 13.00 | 0.002 | CD1A | P06126 | S0232 | −0.58 | 13.45 |
| fusion | | | | | | | | | |
| TNR21 | | | | | | | | | |
| ICAM1 | P05362 | 0.65 | 11.17 | 0.011 | CR2 | P20023 | S0416 | −0.58 | 13.04 |
| DCOR | P11926 | 0.64 | 14.39 | 0.002 | MTA2 | O94776 | T0453 | −0.60 | 13.01 |
| SORL | Q92673 | 0.63 | 11.89 | 0.012 | SPA9 | Q86WD7 | S0063 | −0.60 | 11.03 |
| GELS | P06396 | 0.62 | 11.61 | 0.011 | CD5 | P06127 | S0244 | −0.60 | 13.36 |
| EPHB4 | P54760 | 0.62 | 11.48 | 0.004 | ITAM | P11215 | S0267 | −0.60 | 12.86 |
| RL10A | P62906 | 0.61 | 13.13 | 0.004 | P53 | P04637 | S0047 | −0.61 | 13.95 |
| TACD2 | P09758 | 0.59 | 11.03 | 0.027 | IL2RB | P14784 | S0461 | −0.63 | 11.84 |
| GEMI | O75496 | 0.58 | 14.14 | 0.004 | THYG | P01266 | T0715 | −0.64 | 11.61 |
| EWS | Q01844 | 0.55 | 9.10 | 0.002 | GELS | P06396 | T0869 | −0.64 | 10.76 |
| LGUL | Q04760 | 0.54 | 15.07 | 0.012 | PERM | P05164 | S0395 | −0.65 | 12.79 |
| CAV2 | P51636 | 0.53 | 11.86 | 0.002 | MMP14 | P50281 | S0090 | −0.66 | 11.68 |
| | | | | | VISTA | Q9H7M9 | S0165 | −0.68 | 11.04 |
| | | | | | ANM5 | O14744 | T0038 | −0.76 | 13.11 |
| | | | | | ZDHC6 | Q9H6R6 | T0451 | −0.79 | 13.55 |
| | | | | | LGUL | Q04760 | T0793 | −0.79 | 15.32 |
| | | | | | ALBU | P02768 | T0740 | −0.91 | 13.42 |

TABLE 14
Functional annotations of differentially expressed proteins in mouse corneas, 20 days
after corneal alkali-burn and treatment, between the ‘PBS/control’ and ‘PBS with mC5L2’
treatment group.

| | | Protein | |
|---|---|---|---|
| Accession | Description | Count | Protein Symbols |
| GO.0002376 | immune system | 18 | CD1A, CD5, CD7, CR2, CTSB, CTSD, FCGR3A, |
| | process | | GLO1, ICAM1, ITGAL, MAP2K4, MAPK14, MMP1, |
| | | | MPO, NCOR1, PIR, PPIA, TP53 |
| GO.0001775 | cell activation | 11 | ALB, CD7, CR2, ICAM1, ITGAL, ITGAM, MAPK14, |
| | | | NCOR1, PPIA, PRMT5, TP53 |
| GO.0048731 | system development | 22 | CAV2, CR2, CTSB, EPHB4, FOLR1, GLO1, GMN |
| | | | GSN, GSTM3, ICAM1, ITGAM, MAPK14, MMP14, |
| | | | NCOR1, NEUROD6, ODC1, PIR, PRMT5, SORL1, |
| | | | TACSTD2, TG, TP53 |
| GO.0048513 | organ development | 19 | CAV2, CR2, CTSB, EPHB4, FOLR1, GLO1, GMNN, |
| | | | ICAM1, ITGAM, MAPK14, MMP14, NCOR1, |
| | | | NEUROD6, ODC1, PIR, PRMT5, TACSTD2, TG, |
| | | | TP53 |
| GO.0030198 | extracellular matrix | 8 | CTSB, CTSD, GSN, ICAM1, ITGAL, ITGAM, |
| | organization | | MMP1, MMP14 |
| GO.0002521 | leukocyte | 7 | CR2, GLO1, ITGAM, MAPK14, NCOR1, PIR, TP53 |
| | differentiation | | |
| GO.0046649 | lymphocyte activation | 7 | CD7, CR2, ICAM1, ITGAL, ITGAM, NCOR1, TP53 |
| GO.0007275 | multicellular | 21 | CAV2, CR2, CTSB, EPHB4, FOLR1, GLO1, GMNN, |
| | organismal | | GSN, GSTM3, ICAM1, ITGAM, MAPK14, NCOR1, |
| | development | | NEUROD6, ODC1, PIR, PRMT5, SORL1, TAC |
| | | | STD2, TG, TP53 |
| GO.0042110 | T cell activation | 6 | CD7, ICAM1, ITGAL, ITGAM, NCOR1, TP53 |
| GO.0044707 | single-multicellular | 24 | ALB, CAV2, CD7, CR2, CTSB, CTSD, EPHB4, FOL |
| | organism process | | R1, GLO1, GMNN, GSTM3, ICAM1, ITGAL, MAP |
| | | | K14, MMP1, MPO, NCOR1, NEUROD6, ODC1, PI |
| | | | R, PPIA, SORL1, TG, TP53 |
| GO.0002291 | T cell activation via T | 2 | ICAM1, ITGAL |
| | cell receptor contact | | |
| | with antigen bound to | | |
| | MHC molecule on | | |
| | antigen presenting cell | | |
| GO.0009611 | response to wounding | 9 | ALB, CTSB, FABP5, GSN, ITGAL, ITGAM, MMP |
| | | | 1, PPIA, TP53 |
| GO.0030574 | collagen catabolic | 4 | CTSB, CTSD, MMP1, MMP14 |
| | process | | |
| GO.0070458 | cellular detoxification | 2 | GSTM1, GSTM3 |
| | of nitrogen compound | | |
| GO.0072361 | regulation of | 2 | NCOR1, TP53 |
| | glycolytic process by | | |
| | regulation of | | |
| | transcription from | | |
| | RNA polymerase II | | |
| | promoter | | |
| GO.0007166 | cell surface receptor | 14 | C10orf54, CAV2, CD7, CR2, EPHB4, FCGR3A, IC |
| | signaling pathway | | AM1, IL2RB, ITGAL, ITGAM, MAP2K4, MAPK1 |
| | | | 4, NCOR1, TACSTD2 |
| GO.0050776 | regulation of immune | 9 | C10orf54, CR2, CTSB, FCGR3A, ICAM1, ITGAL, |
| | response | | ITGAM, MAP2K4, MAPK14 |
| GO.0018916 | nitrobenzene | 2 | GSTM1, GSTM3 |
| | metabolic process | | |
| GO.0046651 | lymphocyte | 4 | CR2, ITGAL, ITGAM, TP53 |
| | proliferation | | |
| GO.0006955 | immune response | 11 | CD1A, CD7, CR2, CTSB, FCGR3A, ICAM1, ITGAL, |
| | | | ITGAM, MAP2K4, MAPK14, TP53 |
| GO.0048518 | positive regulation of | 22 | C10orf54, CAV2, CD5, CD53, CR2, CTSB, FCGR3A, |
| | biological process | | ICAM1, ITGAL, ITGAM, MAP2K4, MAPK14, |
| | | | MMP1, MTA2, NCOR1, NEUROD6, ODC1, PPIA, |
| | | | PRMT5, SORL1, TACSTD2, TP53 |
| GO.0032459 | regulation of protein | 3 | MMP1, SORL1, TP53 |
| | oligomerization | | |
| GO.0002573 | myeloid leukocyte | 4 | GLO1, ITGAM, MAPK14, PIR |
| | differentiation | | |
| GO.0006898 | receptor-mediated | 5 | ALB, CAV2, CD5, FOLR1, SORL1 |
| | endocytosis | | |
| GO.0030099 | myeloid cell | 5 | GLO1, ITGAM, MAPK14, NCOR1, PIR |
| | differentiation | | |
| GO.0006575 | cellular modified | 5 | FOLR1, GLO1, GSTM1, GSTM3, TG |
| | amino acid metabolic | | |
| | process | | |
| GO.0006897 | endocytosis | 7 | ALB, CAV2, CD5, FCGR3A, FOLR1, GSN, SORL1 |
| GO.0030097 | hemopoiesis | 7 | CR2, GLO1, ITGAM, MAPK14, NCOR1, PIR, TP53 |
| GO.0042098 | T cell proliferation | 3 | ITGAL, ITGAM, TP53 |
| GO.0044764 | multi-organism | 8 | ALB, CAV2, ICAM1, IL2RB, MMP1, PPIA, RPL10A, |
| | cellular process | | TP53 |
| GO.0007596 | blood coagulation | 7 | ALB, ITGAL, ITGAM, MAPK14, MMP1, PPIA, TP |
| | | | 53 |
| GO.0022617 | extracellular matrix | 4 | CTSB, CTSD, MMP1, MMP14 |
| | disassembly | | |
| GO.0048856 | anatomical structure | 19 | CAV2, CR2, EPHB4, FABP5, FOLR1, GLO1, GMN |
| | development | | N, GSTM3, MAPK14, NCOR1, NEUROD6, ODC1, |
| | | | PIR, PRMT5, RPL10A, SORL1, TACSTD2, TG, TP |
| | | | 53 |
| GO.0050798 | activated T cell | 2 | ITGAL, ITGAM |
| | proliferation | | |
| GO.0006749 | glutathione metabolic | 3 | GLO1, GSTM1, GSTM3 |
| | process | | |
| GO.0002757 | immune response- | 6 | CR2, CTSB, FCGR3A, ITGAM, MAP2K4, MAPK14 |
| | activating signal | | |
| | transduction | | |
| GO.0002224 | toll-like receptor | 4 | CTSB, ITGAM, MAP2K4, MAPK14 |
| | signaling pathway | | |
| GO.0044710 | single-organism | 19 | ALB, CR2, CTSB, CTSD, FOLR1, GLO1, GSN, GS |
| | metabolic process | | TM1, GSTM3, MAP2K4, MMP1, MMP14, MPO, M |
| | | | TA2, NCOR1, ODC1, PIR, PRMT5, SORL1 |
| GO.0090400 | stress-induced | 2 | MAPK14, TP53 |
| | premature senescence | | |
| GO.0030155 | regulation of cell | 7 | C10orf54, CD5, GSN, ICAM1, ITGAL, MMP14, TA |
| | adhesion | | CSTD2 |
| GO.0051701 | interaction with host | 4 | ALB, CAV2, ICAM1, PPIA |
| GO.0007165 | signal transduction | 20 | C10orf54, CAV2, CD53, CD7, CR2, CTSB, EPHB4, |
| | | | FCGR3A, GSN, ICAM1, IL2RB, ITGAL, ITGAM, |
| | | | MAP2K4, MAPK14, NCOR1, SORL1, TACSTD2, |
| | | | TG, TP53 |
| GO.0044403 | symbiosis, | 8 | ALB, CAV2, ICAM1, IL2RB, MMP1, PPIA, RPL10A, |
| | encompassing | | TP53 |
| | mutualism through | | |
| | parasitism | | |
| GO.0002520 | immune system | 7 | CR2, GLO1, ITGAM, MAPK14, NCOR1, PIR, TP53 |
| | development | | |
| GO.0032502 | developmental process | 20 | C10orf54, CAV2, CR2, EPHB4, FABP5, FOLR1, G |
| | | | LO1, GMNN, GSTM3, MAPK14, NCOR1, NEURO |
| | | | D6, ODC1, PIR, PRMT5, RPL10A, SORL1, TACST |
| | | | D2, TG, TP53 |
| GO.0002684 | positive regulation of | 8 | CD5, CR2, CTSB, FCGR3A, ICAM1, ITGAL, ITGA |
| | immune system | | M, MAP2K4 |
| | process | | |
| GO.0009605 | response to external | 12 | ALB, CTSB, EPHB4, GSN, ICAM1, ITGAM, MAP |
| | stimulus | | 2K4, MMP14, MPO, ODC1, TG, TP53 |
| GO.0030260 | entry into host cell | 3 | CAV2, ICAM1, PPIA |
| GO.0043408 | regulation of MAPK | 7 | CAV2, ICAM1, MAP2K4, MAPK14, NCOR1, PR |
| | cascade | | MT5, SORL1 |
| GO.0050896 | response to stimulus | 25 | ALB, C10orf54, CAV2, CD1A, CD53, CD7, CR2, EP |
| | | | HB4, FABP5, FCGR3A, GSTM1, GSTM3, ICAM1, |
| | | | IL2RB, ITGAL, MAP2K4, MAPK14, MMP1, MMP |
| | | | 14, NCOR1, ODC1, PPIA, SORL1, TACSTD2, TP53 |
| GO.0042178 | xenobiotic catabolic | 2 | GSTM1, GSTM3 |
| | process | | |
| GO.0002252 | immune effector | 6 | CR2, FCGR3A, ICAM1, ITGAL, MPO, TP53 |
| | process | | |
| GO.0044763 | single-organism | 31 | ALB, C10orf54, CAV2, CD53, CD7, CR2, CTSB, CT |
| | cellular process | | SD, EPHB4, FCGR3A, FOLR1, GMNN, GSTM1, G |
| | | | STM3, ICAM1, IL2RB, ITGAL, MAP2K4, MAPK1 |
| | | | 4, MMP1, MPO, MTA2, NEUROD6, ODC1, PIR, PP |
| | | | IA, RPL10A, SORL1, TACSTD2, TG, TP53 |
| GO.0048584 | positive regulation of | 12 | C10orf54, CAV2, CR2, CTSB, FCGR3A, GSN, ICA |
| | response to stimulus | | M1, ITGAL, ITGAM, MAP2K4, MAPK14, TP53 |
| GO.0002286 | T cell activation | 3 | ICAM1, ITGAL, TP53 |
| | involved in immune | | |
| | response | | |
| GO.0031334 | positive regulation of | 4 | GSN, ICAM1, MMP1, TP53 |
| | protein complex | | |
| | assembly | | |
| GO.0031065 | positive regulation of | 2 | NCOR1, TP53 |
| | histone deacetylation | | |

PROTEIN SEQUENCES
Sequence 1

| Name: | C5AR2 HUMAN C5a anaphylatoxin chemotactic receptor 2 |
|---|---|
| | (Homo sapiens) |
| Synonyms: | C5L2, GPR77 |
| Organism: | Human |
| Type: | Protein |
| Accession: | NP_060955.1 |
| Length: | 337 |

```
Sequence:
      10 20 30 40 50
MGNDSVSYEY GDYSDLSDRP VDCLDGACLA IDPLRVAPLP LYAAIFLVGV
      60 70 80 90 100
PGNAMVAWVA GKVARRRVGA TWLLHLAVAD LLCCLSLPIL AVPIARGGHW
    110 120 130 140 150
PYGAVGCRAL PSIILLTMYA SVLLLAALSA DLCFLALGPA WWSTVQRACG
    160 170 180 190 200
VQVACGAAWT LALLLTVPSA IYRRLHQEHF PARLQCVVDY GGSSSTENAV
    210 220 230 240 250
TAIRFLFGFL GPLVAVASCH SALLCWAARR CRPLGTAIVV GFFVCWAPYH
    260 270 280 290 300
LLGLVLTVAA PNSALLARAL RAEPLIVGLA LAHSCLNPML FLYFGRAQLR
    310 320 330
RSLPAACHWA LRESQGQDES VDSKKSTSHD LVSEMEV
Sequence 2
```

| | |
|---|---|
| Name: | C5AR1 HUMAN C5a anaphylatoxin chemotactic receptor 1 |
| | (Homo sapiens) |
| Synonyms: | C5AR, C5R1, CD88 |
| Organism: | Human |
| Type: | Protein |
| Accession: | NP_001727.1 |
| Length: | 350 |

```
Sequence:
      10 20 30 40 50
MNSFNYTTPD YGHYDDKDTL DLNTPVDKTS NTLRVPDILA LVIFAVVFLV
      60 70 80 90 100
GVLGNALVVW VTAFEAKRTI NAIWFLNLAV ADFLSCLALP ILFTSIVQHH
    110 120 130 140 150
HWPFGGAACS ILPSLILLNM YASILLLATI SADRFLLVFK PIWCQNFRGA
    160 170 180 190 200
GLAWIACAVA WGLALLLTIP SFLYRVVREE YFPPKVLCGV DYSHDKRRER
    210 220 230 240 250
AVAIVRLVLG FLWPLLTLTI CYTFILLRTW SRRATRSTKT LKVVVAVVAS
    260 270 280 290 300
FFIFWLPYQV TGIMMSFLEP SSPTFLLLNK LDSLCVSFAY INCCINPIIY
    310 320 330 340 350
VVAGQGFQGR LRKSLPSLLR NVLTEESVVR ESKSFTRSTV DTMAQKTQAV
Sequence 3
```

| | |
|---|---|
| Name: | C3aR HUMAN C3a anaphylatoxin chemotactic receptor |
| | (Homo sapiens) |
| Synonyms: | AZ3B, C3R1, C3AR, HNFAG09 |
| Organism: | Human |
| Type: | Protein |
| Accession: | NP_004045.1 |
| Length: | 482 |

```
Sequence:
      10 20 30 40 50
MASFSAETNS TDLLSQPWNE PPVILSMVIL SLTFLLGLPG NGLVLWVAGL
      60 70 80 90 100
KMQRTVNTIW FLHLTLADLL CCLSLPFSLA HLALQGQWPY GRFLCKLIPS
    110 120 130 140 150
IIVLNMFASV FLLTAISLDR CLVVFKPIWC QNHRNVGMAC SICGCIWVVA
    160 170 180 190 200
FVMCIPVFVY REIFTTDNHN RCGYKFGLSS SLDYPDFYGD PLENRSLENI
    210 220 230 240 250
VQPPGEMNDR LDPSSFQTND HPWTVPTVFQ PQTFQRPSAD SLPRGSARLT
    260 270 280 290 300
SQNLYSNVFK PADVVSPKIP SGFPIEDHET SPLDNSDAFL STHLKLFPSA
    310 320 330 340 350
SSNSFYESEL PQGFQDYYNL GQFTDDDQVP TPLVAITITR LVVGFLLPSV
    360 370 380 390 400
IMIACYSFIV FRMQRGRFAK SQSKTFRVAV VVVAVFLVCW TPYHIFGVLS
    410 420 430 440 450
LLTDPETPLG KTLMSWDHVC IALASANSCF NPFLYALLGK DFRKKARQSI
    460 470 480
QGILEAAFSE ELTRSTHCPS NNVISERNST TV
Sequence 4
```

| | |
|---|---|
| Name: | C5AR2 MOUSE C5a anaphylatoxin chemotactic receptor 2 |
| | (Mus musculus) |
| Synonyms: | C5L2, GPR77 |
| Organism: | Mouse |
| Type: | Protein |
| Accession: | NP_795886.2, NP_001139477.1 |
| Length: | 344 |

```
Sequence:
      10 20 30 40 50
MMNHTTSEYY DYEYDHEHYS DLPDVPVDCP AGTCFTSDVY LIVLLVLYAA
      60 70 80 90 100
VFLVGVPGNT LVAWVTWKES RHRLGASWFL HLTMADLLCC VSLPFLAVPI
    110 120 130 140 150
AQKGHWPYGA AGCWLLSSIT ILSMYASVLL LTGLSGDLFL LAFRPSWKGA
    160 170 180 190 200
DHRTFGVRVV QASSWMLGLL LTVPSAVYRR LLQEHYPPRL VCGIDYGGSV
    210 220 230 240 250
SAEVAITTVR FLFGFLGPLV FMAGCHGILQ RQMARRHWPL GTAVVVGFFI
    260 270 280 290 300
CWTPYHVLRV IIAAAPPHSL LLARVLEAEP LFNGLALAHS ALNPIMFLYF
    310 320 330 340
GRKQLCKSLQ AACHWALRDP QDEESAVTKV SISTSHEMVS EMPV
Sequence 5
```

| | |
|---|---|
| Name: | C5AR1 MOUSE C5a anaphylatoxin chemotactic receptor 1 |
| | (Mus musculus) |
| Synonyms: | C5AR, C5R1, CD88 |
| Organism: | Mouse |
| Type: | Protein |
| Accession: | NP_031603.2 |
| Length: | 350 |

```
Sequence:
      10 20 30 40 50
MDPIDNSSFE INYDHYGTMD PNIPADGIHL PKRQPGDVAA LIIYSVVFLV
      60 70 80 90 100
GVPGNALVVW VTAFEARRAV NAIWFLNLAV ADLLSCLALP VLFTTVLNHN
    110 120 130 140 150
YWYFDATACI VLPSLILLNM YASILLLATI SADRFLLVFK PIWCQKVRGT
    160 170 180 190 200
GLAWMACGVA WVLALLLTIP SFVYREAYKD FYSEHTVCGI NYGGGSFPKE
    210 220 230 240 250
KAVAILRLMV GFVLPLLTLN ICYTFLLLRT WSRKATRSTK TLKVVMAVVI
    260 270 280 290 300
CFFIFWLPYQ VTGVMIAWLP PSSPTLKRVE KLNSLCVSLA YINCCVNPII
    310 320 330 340 350
YVMAGQGFHG RLLRSLPSII RNALSEDSVG RDSKTFTPST TDTSTRKSQA
Sequence 6
```

| | |
|---|---|
| Name: | C3AR MOUSE C3a anaphylatoxin chemotactic receptor |
| | (Mus musculus) |
| Synonyms: | AZ3B, C3R1, C3AR, HNFAG09 |
| Organism: | Mouse |
| Type: | Protein |
| Accession: | NP_033909.1 |
| Length: | 477 |

```
Sequence:
      10 20 30 40 50
MESFDADTNS TDLHSRPLFQ PQDIASMVIL GLTCLLGLLG NGLVLWVAGV
      60 70 80 90 100
KMKTTVNTVW FLHLTLADFL CCLSLPFSLA HLILQGHWPY GLFLCKLIPS
    110 120 130 140 150
IIILNMFASV FLLTAISLDR CLIVHKPIWC QNHRNVRTAF AICGCVWVVA
    160 170 180 190 200
FVMCVPVFVY RDLFIMDNRS ICRYNFDSSR SYDYWDYVYK LSLPESNSTD
    210 220 230 240 250
NSTAQLTGHM NDRSAPSSVQ ARDYFWTVTT ALQSQPFLTS PEDSFSLDSA
    260 270 280 290 300
NQQPHYGGKP PNVLTAAVPS GFPVEDRKSN TLNADAFLSA HTELFPTASS
    310 320 330 340 350
GHLYPYDFQG DYVDQFTYDN HVPTPLMAIT ITRLVVGFLV PFFIMVICYS
    360 370 380 390 400
LIVFRMRKTN FTKSRNKTFR VAVAVVTVFF ICWTPYHLVG VLLLITDPES
    410 420 430 440 450
SLGEAVMSWD HMSIALASAN SCFNPFLYAL LGKDFRKKAR QSIKGILEAA
    460 470
FSEELTHSTN CTQDKASSKR NNMSTDV
Conserved Sequence Fragments
Sequence 7
```

| | |
|---|---|
| Organism: | Artificial Sequence |
| Type: | Protein |
| Length: | 14 |

Sequence:
10
FLVGVPGNAM VAWV
Sequence 8

| | |
|---|---|
| Organism: | Artificial Sequence |
| Type: | Protein |
| Length: | 10 |

Sequence:
10
ADLLCCLSLP
Sequence 9

| | |
|---|---|
| Organism: | Artificial Sequence |
| Type: | Protein |
| Length: | 9 |
| Sequence: | |
| 10 | |
| MYASVLLLA | |

Sequence 10

| | |
|---|---|
| Organism: | Artificial Sequence |
| Type: | Protein |
| Length: | 9 |

Sequence:
10
LALLLTVPS
Sequence 11

| | |
|---|---|
| Organism: | Artificial Sequence |
| Type: | Protein |
| Length: | 8 |

Sequence:
10
FFVCWAPY
Sequence 12

| | |
|---|---|
| Organism: | Artificial Sequence |
| Type: | Protein |
| Length: | 6 |
| Sequence: | |
| 10 | |
| GHWPYG | |

Sequence 13

| | |
|---|---|
| Organism: | Artificial Sequence |
| Type: | Protein |
| Length: | 11 |

Sequence:
10
YSDLSDRPVDC
Sequence 14

| | |
|---|---|
| Organism: | Artificial Sequence |
| Type: | Protein |
| Length: | 11 |

Sequence:
10
YSDLPDVPVDC
Sequence 15

| | |
|---|---|
| Organism: | Artificial Sequence |
| Type: | Protein |
| Length: | 9 |
| Sequence: | |
| 10 | |
| TLDLNTPVD | |

Sequence 16

| | |
|---|---|
| Organism: | Artificial Sequence |
| Tvne: | Protein |
| Length: | 9 |

Sequence:
10
TMDPNIPAD
Sequence 17

| | |
|---|---|
| Organism: | Artificial Sequence |
| Type: | Protein |
| Length: | 10 |
| Sequence: | |
| 10 | |
| PLVAITITRL | |

Example Sequences
Sequence 18

| | |
|---|---|
| Organism: | Artificial Sequence |
| Type: | Protein |
| Length: | 23 |
| Other Information: | N-terminal fragment of human C5L2 |

Sequence:
10 20
MGNDSVSYEYGDYSDLSDRPVDC
Sequence 19

| | |
|---|---|
| Organism: | Artificial Sequence |
| Type: | Protein |
| Length: | 29 |
| Other Information: | N-terminal fragment of mouse C5L2 |

Sequence:
10 20
MMNHTTSEYYDYEYDHEHYSDLPDVPVDC
Reference Sequences
Sequence 20

| | |
|---|---|
| Name: | C5A HUMAN, C5a complement component |
| | (Homo sapiens) |
| Synonyms: | C5A |
| Organism: | Human |
| Type: | Protein |
| Accession: | AAA72273.1 |
| Length: | 74 |
| Other Information: | Synthetic construct from human C5 complement |
| | component isoform |

```
Sequence:
      10 20 30 40 50
TLQKK IEEIA AKYKH SVVKK CCYDG ACVNN DETCE QRAAR ISLGP RCIKA
      60 70
FTECC VVASQ LRANI SHKDM QLGR
Sequence 21
```

| | |
|---|---|
| Name: | C5A HUMAN, C5a complement component |
| | (Homo sapiens) |
| Synonyms: | C5A |
| Organism: | Human |
| Type: | Protein |
| Accession: | AAA72273.1 |
| Length: | 73 |
| Other Information: | Synthetic construct from human C5 complement |
| | component isoform |

```
Sequence:
      10 20 30 40 50
TLQKK IEEIA AKYKH SVVKK CCYDG ACVNN DETCE QRAARISLGP RCIKA
      60 70
FTECC VVASQ LRANI SHKDM QLG
Sequence 22
```

| | |
|---|---|
| Organism: | Artificial Sequence |
| Type: | Protein |
| Length: | 14 |

Sequence:
10
TLQKK IEEIA AKYK
Sequence 23

| | |
|---|---|
| Organism: | Artificial Sequence |
| Type: | Protein |
| Length: | 13 |

Sequence:
10
HSVVK KCCYD GAC
Sequence 24

| | |
|---|---|
| Organism: | Artificial Sequence |
| Type: | Protein |
| Length: | 5 |

Sequence:
10
VNNDE
Sequence 25

| | |
|---|---|
| Organism: | Artificial Sequence |
| Type: | Protein |
| Length: | 8 |

Sequence:
10
TCEQRAAR
Sequence 26

| | |
|---|---|
| Organism: | Artificial Sequence |
| Type: | Protein |
| Length: | 4 |

Sequence:
10
ISLG
Sequence 27

| | |
|---|---|
| Organism: | Artificial Sequence |
| Type: | Protein |
| Length: | 22 |

Sequence:
10 20
PRCIK AFTEC CVVAS QLRAN IS
Sequence 28

| | |
|---|---|
| Organism: | Artificial Sequence |
| Type: | Protein |
| Length: | 7 |

Sequence:
10
HKDMQ LG
Sequence 29

| | |
|---|---|
| Organism: | Artificial Sequence |
| Type: | Protein |
| Length: | 8 |

Sequence:
10
HKDMQ LGR
Sequence 30

| | |
|---|---|
| Organism: | Artificial Sequence |
| Type: | Protein |
| Length: | 14 |

Sequence:
10
CCYDG ACVNN DETC
Sequence 31

| | |
|---|---|
| Organism: | Artificial Sequence |
| Type: | Protein |
| Length: | 33 |

Sequence:
10 20 30
CYDGA CVNND ETCEQ RAARI SLGPR CIKAF
Sequence 32

| | |
|---|---|
| Organism: | Artificial Sequence |
| Type: | Protein |
| Length: | 22 |

Sequence:
10 20
CEQRA ARISL GPRCI KAFTE CC
Sequence 33

| | |
|---|---|
| Organism: | Artificial Sequence |
| Type: | Protein |
| Length: | 18 |

Sequence:
10
YDGAC VNNDE TCEQR AAR
Sequence 34

| | |
|---|---|
| Organism: | Artificial Sequence |
| Type: | Protein |
| Length: | 18 |

Sequence:
10
CYDGA CVNND ETCEQ RAA
Sequence 35

| | |
|---|---|
| Organism: | Artificial Sequence |
| Type: | Protein |
| Length: | 9 |

Sequence:
10
X₁X₂ETC EX₃RX₄
Sequence 36

| | |
|---|---|
| Organism: | Artificial Sequence |
| Type: | Protein |
| Length: | 7 |

Sequence:
10
X₅X₆KX₇X₈X₉L
Sequence 37

| | |
|---|---|
| Organism: | Artificial Sequence |
| Type: | Protein |
| Length: | 7 |

Sequence:
10
X₅X₆KX₇X₈X₉I
Sequence 38

| | |
|---|---|
| Organism: | Artificial Sequence |
| Type: | Protein |
| Length: | 7 |

Sequence:
10
NDETC EQRA
Sequence 39

| | |
|---|---|
| Organism: | Artificial Sequence |
| Type: | Protein |
| Length: | 7 |

Sequence:
10
SHKDM QL
Sequence 40

| | |
|---|---|
| Organism: | Artificial Sequence |
| Type: | Protein |
| Length: | 7 |

Sequence:
10
DETCE QR
Sequence 41

| | |
|---|---|
| Organism: | Artificial Sequence |
| Type: | RNA/DNA mixture |
| Length: | 40 |

Sequence:
10 20 30 40
5′-GCGAUG(dU)GGUGGU(dG)(dA)AGGGUUGUUGGG(dU)G(dU)CGACGCA(dC)GC-3′
Sequence 42

| | |
|---|---|
| Organism: | Artificial Sequence |
| Type: | Protein |
| Length: | 7 |

Sequence:
10
KKCCY DG
Sequence 43

| | |
|---|---|
| Name: | C3A HUMAN, C3a complement component |
| | (Homo sapiens) |
| Synonyms: | C3A |
| Organism: | Human |
| Type: | Protein |
| Accession: | AAA72712.1 |
| Length: | 77 |
| Other Information: | Synthetic construct from human C3 complement |
| | component isoform |

```
Sequence:
      10 20 30 40 50
SVQLT EKRMD KVGKY PKELR KCCED GMREN PMRFS CQRRT RFISL GEACK KVFLD
      10 20
CCNYI TELRR QHARA SHLGL AR
Sequence 44
```

| | |
|---|---|
| Organism: | Artificial Sequence |
| Type: | Protein |
| Length: | 8 |

Sequence:
10
ASHLG LAR
Sequence 45

| | |
|---|---|
| Organism: | Artificial Sequence |
| Type: | Protein |
| Length: | 9 |

Sequence:
10
ASHLG LARG
Sequence 46

| | |
|---|---|
| Organism: | Artificial Sequence |
| Type: | Protein |
| Length: | 13 |

Sequence:
10
RQHAR ASHLGLAR
Sequence 47

| | |
|---|---|
| Organism: | Artificial Sequence |
| Type: | Protein |
| Length: | 14 |

Sequence:
10
RQHAR ASHLGLARG
Sequence 48

| | |
|---|---|
| Name: | C4A HUMAN, C4a complement component |
| | (Homo sapiens) |
| Synonyms: | C4A |
| Organism: | Human |
| Type: | Protein |
| Accession: | AAB59537.1 |
| Length: | 77 |
| Other Information: | Synthetic construct from human C4 complement |
| | component isoform |

```
Sequence:
      10 20 30 40 50
NVNFQ KAINE KLGQY ASPTA KRCCQ DGVTR LPMMR SCEQR AARVQ QPDCR
      10 20
EPFLS CCQFA ESLRK KSRDK GQAGL QR
Sequence 49
```

| | |
|---|---|
| Name: | C4A HUMAN, C4a complement component |
| | (Homo sapiens) |
| Synonyms: | C4A |
| Organism: | Human |
| Type: | Protein |
| Accession: | AAB59537.1 |
| Length: | 380 |

```
Sequence:
      10 20 30 40 50
TLEIP GNSDP NMIPD GDFNS YVRVT ASDPL DTLGS EGALS PGGVA SLLRL
      60 70 80 90 100
PRGCG EQTMI YLAPT LAASR YLDKT EQWST LPPET KDHAV DLIQK GYMRI
    110 120 130 140 150
QQFRK ADGSY AAWLS RDSST WLTAF VLKVL SLAQE QVGGS PEKLQ ETSNW
    160 170 180 190 200
LLSQQ QADGS FQDPC PVLDR SMQGG LVGND ETVAL TAFVT IALHH GLAVF
    210 220 230 240 250
QDEGA EPLKQ RVEAS ISKAN SFLGE KASAG LLGAH AAAIT AYALS LTKAP
    210 220 230 240 250
VDLLG VAHNN LMAMA QETGD NLYWG SVTGS QSNAV SPTPA PRNPS DPMPQ
    310 320 330 340 350
APALW IETTA YALLH LLLHE GKAEM ADQAS AWLTR QGSFQ GGFRS TQDTV
    360 370
IALDA LSAYW IASHT TEERG LNVTL SSTGR
Sequence 50
```

| | |
|---|---|
| Organism: | Artificial Sequence |
| Type: | Protein |
| Length: | 6 |

Sequence:
10
PCPVL D

1. Binder binding to complement-anaphylatoxin C5a and/or C3a and/or C4a and thereby preferably inhibiting the activity of C5a and/or C3a and/or C4a for use in the treatment of a subject having an ocular wound and/or fibrosis.

2. Binder for use in the treatment of a subject having an ocular wound and/or fibrosis according to claim 1 wherein said binder is selected from the group comprising a protein or a fragment thereof, a peptide, a non-IgG scaffold, an aptamer, oligonucleotides, an antibody or antibody-like proteins, peptidomimetics or a fragment thereof.

3. Binder according to claim 1 for use in the treatment of a subject having an ocular wound and/or fibrosis wherein said binder is administered to promote wound healing, in particular corneal wound healing.

4. Binder according to claim 1 for use in the treatment of a subject having an ocular wound and/or fibrosis, wherein said binder may bind to several overlapping peptide fragments of a complement component C5a protein having the amino acid sequence depicted in SEQ ID No.: 20 or SEQ ID No.: 21, wherein overlapping means the overlapping of the targeted amino acid sequences of the antibody, antibody-like protein or binder and the specific peptide fragments.

5. Binder according to claim 4 for use in the treatment of a subject having an ocular wound and/or fibrosis, wherein said binder may bind only to C5a at an epitope within or overlapping with a fragment of the protein having the amino acid sequence, according to SEQ ID No's.: 22-34.

6. Binder according to claim 4 for use in the treatment of a subject having an ocular wound and/or fibrosis, wherein said binder may also bind to an epitope of C5a formed by amino acid sequences according to SEQ ID No's: 35-40 (SEQ ID No.: 35: X₁X₂ETCEX₃RX₄, SEQ ID No.: 36: X₅X₆KX₇X₈X₉L and SEQ ID No.: 37: X₅X₆KX₇X₈X₉I), wherein X₁ is selected from the group consisting of N, H, D, F, K, Y, and T; X₂ is selected from the group consisting of D, L, Y, and H; X₃ is selected from the group consisting of Q, E, and K; X₄ is selected from the group consisting of A, V, and L; X₅ is selected from the group consisting of S, H, P, and N; X₆ is selected from the group consisting of H and N; X₇ is selected from the group consisting of D, N, H, P, and G; X₈ is selected from the group consisting of M, L, I, and V; and X₉ is selected from the group consisting of Q, L, and I.

7. Binder according to claim 1 for use in the treatment of a subject having an ocular wound and/or fibrosis, wherein said binder may bind to several overlapping peptide fragments of a complement component C3a protein having the amino acid sequence depicted in SEQ ID No.: 43, and preferably wherein said binder may also bind only to a human C3a at an epitope within or overlapping with a fragment of the protein having the amino acid sequence, according to SEQ ID No's.: 44-47.

8. (canceled)

9. Binder according to claim 1 for use in the treatment of a subject having an ocular wound and/or fibrosis, wherein said binder may bind to several overlapping peptide fragments of a complement component C4a protein having the amino acid sequence depicted in SEQ ID No.: 48 or SEQ ID No.: 49, and preferably wherein said binder may also bind only to a human C4a at an epitope within or overlapping with a fragment of the protein having the amino acid sequence, according to SEQ ID No.: 50.

10. (canceled)

11. Binder according to claim 1 for use in the treatment of a subject having an ocular wound and/or fibrosis, wherein said binder is an antibody or an antibody-like protein or an aptamer.

12. (canceled)

13. Binder according to claim 12 for use in the treatment of a subject having an ocular wound and/or fibrosis, wherein said binder is an aptamer, wherein said aptamer may relate to a nucleic acid molecule consisting of RNA and/or DNA, such as disclosed in SEQ ID No.: 41, and preferably wherein said aptamer binds to a binding site on C5a comprising SEQ ID No: 42.

14. (canceled)

15. Binder according to claim 1 for use in the treatment of a subject having an ocular wound and/or fibrosis wherein said binder is a protein or protein fragment is selected from the group comprising human C5L2 protein according to SEQ ID No.: 1, a protein/peptide or fragment that is at least 60% identical to the full-length amino acid sequence of human C5L2 protein of SEQ ID No.:1, human C5aR1 protein according to SEQ ID No.: 2, a protein or fragment that is at least 60% identical to the full-length amino acid sequence of human C5aR1 protein of SEQ ID No.: 2, human C3aR protein according to SEQ ID No.: 3, a protein or fragment that is at least 60% identical to the full-length amino acid sequence of human C3aR protein as of SEQ ID No.: 3, a mouse C5L2 protein according to SEQ ID No.: 4, a protein or fragment that is at least 60% identical to the full-length amino acid sequence of mouse C5L2 protein of SEQ ID No.:4, mouse C5aR1 protein according to SEQ ID No.: 5, a protein or fragment that is at least 60% identical to the full-length amino acid sequence of mouse C5aR1 protein of SEQ ID No.: 5, mouse C3aR protein according to SEQ ID No.: 6, and a protein or fragment that is at least 60% identical to the full-length amino acid sequence of mouse C3aR protein of SEQ ID No.: 6.

16. Binder according to claim 1 for use in the treatment of a subject having an ocular wound and/or fibrosis wherein said binder is a protein/peptide or protein fragment and comprises at least one conserved region, preferably at least two conserved regions, selected from the group comprising an amino acid sequence according to SEQ ID No.:7, an amino acid sequence according to SEQ ID No.:8, an amino acid sequence according to SEQ ID No.:9, an amino acid sequence according to SEQ ID No.:10, an amino acid sequence according to SEQ ID No.:11, an amino acid sequence according to SEQ ID No.:12, an amino acid sequence according to SEQ ID No.:13, an amino acid sequence according to SEQ ID No.:14, an amino acid sequence according to SEQ ID No.:15, an amino acid sequence according to SEQ ID No.:16, an amino acid sequence according to SEQ ID No.:17, and a protein or fragment that is at least 60% identical to any of the amino acid sequences according to SEQ ID No's.:7-17.

17. (canceled)

18. Binder according to claim 15 for use in the treatment of a subject having an ocular wound and/or fibrosis wherein said binder is a protein/peptide or protein fragment and comprises at least one conserved region selected from the group comprising an amino acid sequence according to SEQ ID No.:18 and an amino acid sequence according to SEQ ID No.:19.

19. Composition comprising at least two binders, preferably proteins or protein fragments, more preferably at least three proteins fragments, according to claim 1 for use in the treatment of a subject having an ocular wound and/or fibrosis.

20. (canceled)

21. A method for the treatment of a subject having an ocular wound and/or fibrosis, comprising administering to said subject a pharmaceutical composition comprising a binder according to claim 1.

22. A method for the treatment of a subject wherein said subject suffers from a disease selected from the group comprising: conjunctivitis and conjunctival scars (including ocular pemphigoid), scleritis and episcleritis, corneal scars and opacities due to corneal ulcer, keratoconjunctivitis, keratitis, bullous keratopathy, corneal degenerations, iridocyclitis and adhesions of iris and ciliary body, chorioretinal scars/fibrosis due to chorioretinal inflammation or degeneration or haemorrhage or rupture or neovascularization, fibrotic vitreoretinopathies, such as in proliferative vitreoretinopathy, retinopathy of prematurity and diabetic retinopathy; choroidal neovascularization and degenerations of the macula, secondary glaucoma, endophthalmitis, and impairments of wound healing and fibrosis after ocular surgery or trauma, including intraocular foreign bodies, (idiopathic) pulmonary fibrosis, dermal keloid formation, scleroderma, myelofibrosis, kidney-, pancreas- and heart-fibrosis, and fibrosis in (non)-alcoholic steatohepatosis, glomerulonephritis and (ANCA-associated) vasculitis, comprising administering to said subject a pharmaceutical composition comprising a binder according to claim 1.

23. (canceled)
